(19) United States
(12) Patent Application Publication
(10) Pub. No.: US 2016/0143910 A1
(43) Pub. Date: May 26, 2016

(54) METHODS OF TREATING CANCER AND PREVENTING CANCER DRUG RESISTANCE

(71) Applicants: Shilpi ARORA, Cambridge, MA (US); Michael Robert COSTA, South San Francisco, CA (US); Ted LAU, South San Francisco, CA (US); Patrick TROJER, Cambridge, MA (US); GENENTECH, INC., South San Francisco, CA (US); CONSTELLATION PHARMACEUTICALS, INC., Cambridge, MA (US)

(72) Inventors: Shilpi Arora, Cambridge, MA (US); Michael Robert Costa, South San Francisco, CA (US); Ted Lau, South San Francisco, CA (US); Patrick Trojer, Cambridge, MA (US); Brian K. Albrecht, Cambridge, MA (US); Shane Buker, Cambridge, MA (US); Marie Classon, South San Francisco, CA (US); Victor S. Gehling, Cambridge, MA (US); Jean-Christophe Harmange, Cambridge, MA (US); Erica L. Jackson, South San Francisco, CA (US); Jun Liang, South San Francisco, CA (US); Heidi Phillips, South San Francisco, CA (US); Peter Sandy, Cambridge, MA (US); Jeffrey Settleman, South San Francisco, CA (US); Jean-Philippe Stephan, South San Francisco, CA (US)

(73) Assignees: CONSTELLATION PHARMACEUTICALS, INC., Cambridge, MA (US); GENENTECH, INC., South San Francisco, CA (US)

(21) Appl. No.: 14/776,586

(22) PCT Filed: Mar. 14, 2014
(86) PCT No.: PCT/US14/29432

§ 371 Date: Sep. 14, 2015

Related U.S. Application Data
(60) Provisional application No. 61/801,414, filed on Mar. 15, 2013.
(60) Provisional application No. 61/804,083, filed on Mar. 21, 2013.

(51) Int. Cl. A61K 31/519; A61K 31/437; A61K 31/517; A61K 45/06; A61K 31/337

(52) U.S. Cl. CPC A61K 31/519; A61K 45/06; A61K 31/337; A61K 31/517; A61K 31/437

(57) ABSTRACT

Provided herein are methods of treating and/or preventing cancer drug resistance using antagonists of KDM5.

### CROSS-REFERENCE TO RELATED APPLICATIONS

This patent application claims the benefit of priority of U.S. application Ser. No. 61/801,414, filed Mar. 15, 2013, and of U.S. application Ser. No. 61/804,083, filed Mar. 21, 2013, which applications are herein incorporated by reference.

### FIELD

Provided herein are methods of treating and/or preventing cancer drug resistance using antagonists of KDM5 as described herein.

### BACKGROUND

The relatively rapid acquisition of resistance to cancer drugs remains a key obstacle to successful cancer therapy. Substantial efforts to elucidate the molecular basis for such drug resistance have revealed a variety of mechanisms, including drug efflux, acquisition of drug binding-deficient mutants of the target, engagement of alternative survival pathways, and epigenetic alterations. Such mechanisms are generally believed to reflect the existence of rare, stochastic, resistance-conferring genetic alterations within a tumor cell population that are selected during drug treatment. See Sharma et al., Cell 141(1):69-80 (2010). An increasingly observed phenomenon in cancer therapy is the so-called “re-treatment response.” For example, some non-small cell lung cancer (NSCLC) patients who respond well to treatment with EGFR (epidermal growth factor receptor) tyrosine kinase inhibitors (TKIs), and who later experience therapy failure, demonstrate a second response to EGFR™ re-treatment after a “drug holiday.” See Kurata et al., Ann. Oncol. 15:173-174 (2004); Yano et al., Oncol. Res. 15:107-111 (2005). Similar re-treatment responses are well established for several other cancer therapy agents. See Cara and Tannock, Ann. Oncol. 12:23-27 (2001). Such findings suggest that acquired resistance to cancer drugs may involve a reversible “drug-tolerant” state, whose mechanistic basis remains to be established.

While some specific resistance-conferring mutations have indeed been identified in many cancer patients demonstrating acquired drug resistance, the relative contribution of mutational and non-mutational mechanisms to drug resistance, and the role of tumor cell subpopulations remain somewhat unclear. New treatment methods are needed to successfully address heterogeneity within cancer cell populations and the emergence of cancer cells resistant to drug treatments.

### SUMMARY

Provided herein are methods of using antagonists of KDM5, for example, for treating cancer and/or preventing drug resistance in an individual. For example, a method of treating cancer in an individual comprising administering to the individual an antagonist of KDM5 alone or in combination with a cancer therapy agent. In some embodiments, the individual is selected for treatment with a cancer therapy agent (e.g., targeted therapies, chemotherapies, and/or radiotherapies). In some embodiments, the individual starts treatment comprising administration of an antagonist of KDM5 prior to treatment with the cancer therapy agent. In some embodiments, the individual concurrently receives treatment comprising the antagonist of KDM5 and the cancer therapy agent. In some embodiments, the antagonist of KDM5 increases the period of cancer sensitivity and/or delays development of cancer resistance.

In another aspect provided herein are combination therapies using antagonists of KDM5 and cancer therapy agents (e.g., targeted therapies, chemotherapies, and/or radiotherapies).

In particular, provided herein are methods of treating cancer in an individual comprising administering to the individual (a) an antagonist of KDM5 and (b) a cancer therapy agent (e.g., targeted therapy, chemotherapy, and/or radiotherapy). In some embodiments, the respective amounts of the antagonist of KDM5 and the cancer therapy agent are effective to increase the period of cancer sensitivity and/or delay the development of cancer cell resistance to the cancer therapy agent. In some embodiments, the respective amounts of the antagonist of KDM5 and the cancer therapy agent are effective to increase efficacy of a cancer treatment comprising the cancer therapy agent. For example, in some embodiments, the respective amounts of the antagonist of KDM5 and the cancer therapy agent are effective to increase efficacy compared to a treatment (e.g., standard of care treatment) (e.g., standard of care treatment) comprising administering an effective amount of the cancer therapy agent without (in the absence of) the antagonist of KDM5. In some embodiments, the respective amounts of the antagonist of KDM5 and the cancer therapy agent are effective to increase response (e.g., complete response) compared to a treatment (e.g., standard of care treatment) comprising administering an effective amount of cancer therapy agent without (in the absence of) the antagonist of KDM5.

Also provided herein are methods of increasing efficacy of a cancer treatment comprising a cancer therapy agent in an individual comprising administering to the individual (a) an effective amount of an antagonist of KDM5 and (b) an effective amount of the cancer therapy agent.

Provided herein are methods of treating cancer in an individual wherein cancer treatment comprising administering to the individual (a) an effective amount of an antagonist of KDM5 and (b) an effective amount of a cancer therapy agent, wherein the cancer treatment has increased efficacy compared to a treatment (e.g., standard of care treatment) comprising administering an effective amount of cancer therapy agent without (in the absence of) the antagonist of KDM5.

In addition, provided herein are methods of delaying and/or preventing development of cancer resistant to a cancer therapy agent in an individual, comprising administering to the individual (a) an effective amount of an antagonist of KDM5 and (b) an effective amount of the cancer therapy agent.

Provided herein are methods of treating an individual with cancer who has an increased likelihood of developing resistance to a cancer therapy agent comprising administering to the individual (a) an effective amount of an antagonist of KDM5 and (b) an effective amount of the cancer therapy agent.

Further provided herein are methods of increasing sensitivity to a cancer therapy agent in an individual with cancer comprising administering to the individual (a) an effective amount of an antagonist of KDM5 and (b) an effective amount of the cancer therapy agent.

Provided herein are also methods of extending the period of a cancer therapy agent sensitivity in an individual with cancer comprising administering to the individual (a) an effective amount of an antagonist of KDM5 and (b) an effective amount of the cancer therapy agent.

Provided herein are methods of extending the duration of response to a cancer therapy agent in an individual with cancer comprising administering to the individual (a) an effective amount of an antagonist of KDM5 and (b) an effective amount of the cancer therapy agent.

In some embodiments of any of the methods, the cancer therapy agent is a targeted therapy. In some embodiments, the targeted therapy is one or more of an EGFR antagonist, RAF inhibitor, and/or PI3K inhibitor.

In some embodiments of any of the methods, the targeted therapy is an EGFR antagonist. In some embodiments of any of the methods, the EGFR antagonist is N-(3-ethynylphenyl)-6,7-bis(2-methoxyethoxy)-4-quinazolinamine and/or a pharmaceutical acceptable salt thereof. In some embodiments, the EGFR antagonist is N-(3-ethynylphenyl)-6,7-bis(2-methoxyethoxy)-4-quinazolinamine. In some embodiments, the EGFR antagonist is N-(4-(3-fluorobenzyloxy)-3-chlorophenyl)-6-(5-((2-(methylsulfonyl)ethylamino)methyl)furan-2-yl)quinazolin-4-amine,di4-methylbenzenesulfonate or a pharmaceutically acceptable salt thereof (e.g., lapatinib).

In some embodiments of any of the methods, targeted therapy is a RAF inhibitor. In some embodiments, the RAF inhibitor is a BRAF inhibitor. In some embodiments, the RAF inhibitor is a CRAF inhibitor. In some embodiments, the BRAF inhibitor is vemurafenib. In some embodiments, the RAF inhibitor is 3-(2-cyanopropan-2-yl)-N-(4-methyl-3-(3-methyl-4-oxo-3,4-dihydroquinazolin-6-ylamino)phenyl)benzamide or a pharmaceutically acceptable salt thereof (e.g., AZ628 (CAS#878739-06-1)).

In some embodiments of any of the methods, the targeted therapy is a PI3K inhibitor.

In some embodiments of any of the methods, the cancer therapy agent is chemotherapy. In some embodiments of any of the methods, the chemotherapy is a taxane. In some embodiments, the taxane is paclitaxel. In some embodiments, the taxane is docetaxel.

In some embodiments of any of the methods, the chemotherapy is a platinum agent. In some embodiments, the platinum agent is carboplatin. In some embodiments, the platinum agent is cisplatin. In some embodiments of any of the methods, the chemotherapy is a taxane and a platinum agent. In some embodiments, the taxane is paclitaxel. In some embodiments, the taxane is docetaxel. In some embodiments, the platinum agent is carboplatin. In some embodiments, the platinum agent is cisplatin.

In some embodiments of any of the methods, the chemotherapy is a vinca alkyloid. In some embodiments, the vinca alkyloid is vinorelbine. In some embodiments of any of the methods, the chemotherapy is a nucleoside analog. In some embodiments, the nucleoside analog is gemcitabine.

In some embodiments of any of the methods, the cancer therapy agent is radiotherapy.

In some embodiments of any of the methods, the antagonist of KDM5 is a KDM5 small molecule antagonist.

Examples of small molecule antagonists of KDM5 that may be useful in the practice of certain embodiments include compounds of Formula I or II, an isomer or a mixture of isomers thereof or a pharmaceutically acceptable salt, solvate or prodrug thereof. Such compounds, and processes and intermediates that are useful for preparing such compounds, are described in WO 2012/007007 and WO 2012/007008.

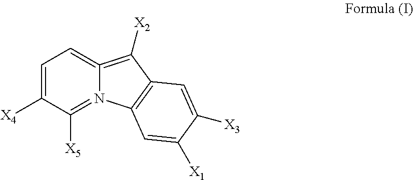

Wherein X₁ represents -A-B, wherein
A represents a bond, O, S or NH, and
B represents
C1-6-alkyl, C2-4-alkenyl or C2-4-alkynyl,

which C1-6-alkyl, C2-4-alkenyl or C2-4-alkynyl may optionally be substituted with one or more substituents selected from the group consisting of hydroxy, C3-6-cycloalkyl, C1-4-alkoxy, hydroxy-C1-4-alkoxy, halo, trifluoromethyl, —NH₂, methylamino, dimethylamino, sulfamoyl, dimethylsulfamoyl, methylsulfonyl, methylsulfonyloxo, methylsulfinyl, methylsulfanyl, cyano, —(C═O)R′, a phenyl group, and a monocyclic or bicyclic heterocyclic group,

where

R′ represents hydroxy, C1-4-alkyl, halo-C1-4-alkyl, C1-4-alkoxy, —NH₂, methylamino, dimethylamino, a phenyl group or a monocyclic or bicyclic heterocyclic group;

and where

the phenyl group may be substituted with one or more of the substituents selected from the group consisting of methyl, trifluoromethyl, halo, cyano, acetamino, methylsulfonylamino, and a monocyclic or bicyclic heterocyclic group;

### —OH or —(C═O)R″,

where R″ represents hydrogen, hydroxy, C1-4-alkyl, cyclopropyl, halo-C1-4-alkyl, C1-4-alkoxy, —COOH, —NH₂, methylamino, dimethylamino, methylsulfonyl, or a monocyclic or bicyclic heterocyclic group; or

where R″ represents C1-4-alkyl, C1-4-alkoxy, oxy, carbamoyl, amine or a monocyclic or bicyclic heterocyclic group, which is substituted with one or more substituents selected from the group consisting of hydroxy, methyl, ethyl, —O—C1-6-alkyl, hydroxymethyl, hydroxymethyl, methoxyethyl, acetyl, cyano, ethoxycarbonyl, dimethylamino, N-[3(dimethylamino)propyl]N′ethylcarbamimidoyl, methylsulfinyl, methylsulfanyl, methylsulfonyl, methoxyethoxyethyl, (dimethylamino)ethyl and methylsulfanylethyl, which —O—C1-6-alkyl may optionally be substituted with hydroxy, methoxy or dimethylamino;

### —(C═S)R′″,

where R′″ represents —NH₂, methylamino or dimethylamino;

### —C(CH3)=N—R″″,

where R″″ represents hydroxy or methoxy;

sulfamoyl, dimethylsulfamoyl, sulfinyl or sulfonyl,

which sulfamoyl, sulfonyl or sulfonyl may optionally be substituted with one or more substituents selected from the group consisting of C1-4-alkyl, halo-C1-4-alkyl, methoxy-C1-4-alkyl, dimethylamino, (dimethylamino)methyl, (dimethylamino)ethyl, C3-6-cycloalkyl, C2-4-alkenyl and a monocyclic or bicyclic heterocyclic group;

fluoro, chloro, bromo or cyano; or
a monocyclic or bicyclic heterocyclic group,
where the monocyclic or bicyclic heterocyclic group may optionally be substituted with one or more substituents selected from the group consisting of C1-2-alkyl, halo, halo-C1-2-alkyl, C1-4-alkoxy, C1-4-alkoxycarbonyl, COOH, cyano, —NH₂, methylamino and dimethylamino;
and
X₂ represents
C1-18-alkyl, C2-18-alkenyl, or C2-18-alkynyl,

which C1-18-alkyl, C2-18-alkenyl, C2-18-alkynyl may optionally be substituted with one or more substituents selected from the group consisting of C3-6-cycloalkyl, hydroxy, halo, trifluoromethyl, C1-6-alkoxy, hydroxy-C1-6-alkoxy, C1-6-alkyl-C1-6-alkoxy, trifluoromethyi-C1-6-alkoxy, oxo-C1-6-alkyl, —NH₂, dimethylamino, cyano, phenyl, a 5-membered monocyclic heterocyclic group, or a 6-membered monocyclic heterocyclic group, which phenyl, 5-membered monocyclic heterocyclic group, or 6-membered monocyclic heterocyclic group may optionally be substituted with one or more substituents selected from the group consisting of C1-6-alkyl or halo; or

—O-Xa, —(C═O)—O-Xb, —(C═O)-Xc, —NXd1Xd2, —(CO)—NXE1XE2, or —(C═O)—NH—SO₂-Xf, where Xa, Xb, Xc, Xd1, Xd2, Xe1, Xe2 or Xf independently of each other represent hydrogen, C1-18-alkyl, C2-18-alkenyl, C2-18-alkynyl, C3-6-cycloalkyl, phenyl, a 5-membered monocyclic heterocyclic group or a 6-membered monocyclic heterocyclic group;

which C1-18-alkyl, C2-18-alkenyl, C2-18-alkynyl, C3-6-cycloalkyl, phenyl, a 5-membered monocyclic heterocyclic group or a 6-membered monocyclic heterocyclic group may optionally be substituted with one or more substituents selected from the group consisting of C3-6-cycloalkyl, hydroxy, halo, trifluoromethyl, C1-4-alkyl, C1-6-alkoxy, C1-6-alkoxycarbonyl, C1-4-alkylamino, hydroxy-C1-6-alkoxy, C1-6-alkyl-C1-6-alkoxy, trifluoro-C1-6-alkoxy, trifluoromethyl-O—C1-6-alkyl, oxo-C1-6-alkyl, —NH₂, methylamino, dimethylamino, (methoxyethyl)(methyl)amino, [(dimethylamino)ethyl](methyl)amino, cyano, —O—C1-6-alkyl-phenyl, phenyl, a 5-membered monocyclic heterocyclic group, or a 6-membered monocyclic heterocyclic group, which phenyl, 5-membered monocyclic heterocyclic group, or 6-membered monocyclic heterocyclic group may optionally be substituted with one or more substituents selected from the group consisting of C1-6-alkyl, —(C═O)—O—C1-6-alkyl or halo; or

where Xe1 and Xe2 independently of each other represent hydrogen, hydroxy, C1-18-alkyl, C2-18-alkenyl, C2-18-alkynyl, C3-6-cycloalkyl, —O—C1-6-alkyl, phenyl, a 5-membered monocyclic heterocyclic group or a 6-membered monocyclic heterocyclic group,

which —O—C1-6-alkyl may optionally be substituted with hydroxy, methoxy, or dimethylamino;

with the proviso in certain embodiments that Xe1 and Xe2 cannot both represent hydrogen; and with the proviso in certain embodiments that Xb cannot represent hydrogen; or

### —(C═O)—O—CH2-CH2-NXj1Xj2, —S-Xk, —(C═S)—N(CH3)-Xm or —(C═S)—N-Xn,

where Xj1, Xj2, Xk, Xm, Xn independently of each other represent methyl, ethyl, propyl, amino, methylamino or dimethylamino,

which methyl, ethyl or propyl may optionally be substituted with one or more substituents selected from the group consisting of methoxycarbonyl, dimethylamino, carbamoyl, phenyl, cyanophenyl, and a 5- or 6-membered monocyclic heterocyclic group; and

X₃ represents hydrogen, C1-4-alkyl, C2-4-alkenyl, C2-4-alkynyl or
—O-Xg —S-Xh or —NXi1Xi2 where X9g, Xh, Xi1 and Xi2 independently of each other represent hydrogen, —(CH2)n-CH3, or —(CH2)n-COOH, where n is 0, 1, 2, 3 or 4
and
X₄ and X₅ independently of each other represent
hydrogen, C1-4-alkyl, halo-C1-4-alkyl, C3-6-cycloalkyl, halo, nitro, —NH₂, or cyano.

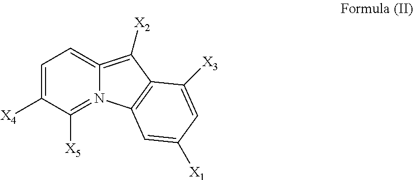

### Wherein

X₁ represents -A-B, wherein
A represents a bond, O, S, or NH, and
B represents
C1-6-alkyl, C2-4-alkenyl, C2-4-alkynyl or C3-5-cycloalkyl which C1-6-alkyl, C2-4-alkenyl,
C2-4-alkynyl or C3-5-cycloalkyl may optionally be substituted with one or more substituents selected from the group consisting of hydroxy, C3-6-cycloalkyl, C1-4-alkoxy, hydroxy-C1-4-alkoxy, —NH₂, methylamino, dimethylamino, sulfamoyl, dimethylsulfamoyl, methylsulfonyl, methylsulfonyloxo, cyano, —(C═O)R′, a phenyl group, and a monocyclic or bicyclic heterocyclic group,

where

R′ represents hydroxy, C1-4-alkyl, halogen-C1-4-alkyl, C1-4-alkoxy, —NH₂, methylamino, cyclopropyl, dimethylamino, a phenyl group or a monocyclic or bicyclic heterocyclic group;

and where

the phenyl group may be substituted with one or more of the substituents selected from the group consisting of methyl, trifluoromethyl, halogen, cyano, acetamino, methylsulfonylamino, and a monocyclic or bicyclic heterocyclic group; or

—OH, with the proviso in certain embodiments that B only represent —OH, when A is a bond; or

### or —(C═O)R″,

where R″ represents hydroxy, halogen-C1-4-alkyl, C1-4-alkoxy, hydroxy-C1-4-alkoxy, —NH₂, C1-3-alkyl-amino, di-C1-3-alkyl-amino, methylsulfonyl, a monocyclic or bicyclic heterocyclic group, C3-4-cycloalkyl or C1-4-alkyl, wherein said C3-4-cycloalkyl or C1-4 alkyl optionally may be substituted with one or more substituents selected from the group consisting of hydroxy, C3-6-cycloalkyl, C1-3-alkoxy, hydroxy-C1-3-alkoxy, —NH₂, methylamino, dimethylamino, 6 membered heterocyclic ring, sulfamoyl, dimethylsulfamoyl, methylsulfonyl, methylsulfonyloxo, cyano, —(C═O)R′, a halo-phenyl group, and a monocyclic or bicyclic heterocyclic group, where R′ is as identified above;

### —(C═O)NH—R′″,

where R′″ represents hydroxyethyl, methoxyethyl, dimethylaminoethyl, methanesulfonyl or —O—C1-6-alkyl optionally substituted with dimethylamino;

sulfamoyl, sulfinyl, sulfanyl or sulfonyl,

which sulfamoyl may optionally be substituted with one or two C1-3-alkyl groups and said sulfinyl, sulfanyl or sulfonyl may optionally be substituted with one substituent selected from the group consisting of C1-4-alkyl, halogen-C1-4-alkyl, carbonyi-C1-3-alkyl, methylsulfamoyl, C3-6-cycloalkyl, C1-3-alkyl-amino, di-C1-3-alkyl-amino, dimethylaminoethyl, a 6 membered heterocyclic ring, and a monocyclic or bicyclic heterocyclic group;

a phenyl, monocyclic or bicyclic heterocyclic group,

where the phenyl, monocyclic or bicyclic heterocyclic group may optionally be substituted with one or more substituents selected from the group consisting of halogen, halogen-C1-3-alkyl, C1-3-alkoxy, C1-3-alkoxyalkoxy, C1-3-alkoxycarbonyl, COOH, cyano, —NH₂, methylamino, dimethylamino, cyclopropyl and C1-3-alkyl, wherein said cyclopropyl or C1-3 alkyl optionally may be substituted with one or more substituents selected from the group consisting of hydroxy, cyclopropyl, C1-3-alkoxy, hydroxy-C1-3-alkoxy, —NH₂, methylamino, dimethylamino, 6 membered heterocyclic ring, sulfamoyl, dimethylsulfamoyl, methylsulfonyl, methylsulfonyloxo, cyano, —(C═O)R′, a halogen-phenyl group, and a monocyclic or bicyclic heterocyclic group, where R′ is as identified above;

and
X₂ represents

—COOH, (C═O)NH₂ or —CN

and
X₃ represents

Hydrogen or —OH; or

—Y-Xa-Xb

where

Y is O, C═O or a bond; and

Xa is -a bond, C1-18-alkyl, C2-18-alkenyl, C2-18-alkynyl, C3-10-cycloalkyl, —C1-18-alkylO-, —O— or —NXb-, with the proviso in certain embodiments that when Y is O then Xa is not 0; and each Xb is individually —H, C3-6-cycloalkyl, C1-6 alkoxy, phenyl, phenoxy, a 5-membered monocyclic heterocyclic group, a 6-membered monocyclic heterocyclic group or a bicyclic heteroaromatic group, which C3-10-cycloalkyl, C1-6 alkoxy, phenyl, phenoxy, 5-membered monocyclic heterocyclic group, 6-membered monocyclic heterocyclic group or bicyclic heteroaromatic group may optionally be substituted with one or more substituents selected from the group consisting of halogen, halogen-C1-4-alkyl, hydroxy linear or branched C1-4-alkoxy, C1-6-alkoxyalkoxy, C1-4-alkoxycarbonyl, C1-4-alkylcarbonyl, COOH, cyano, —NH₂, methylamino, dimethylamino, hydroxy and linear or branched C1-5-alkyl, wherein said C1-5 alkyl optionally may be substituted with one or more substituents selected from the group consisting of hydroxy, C3-6-cycloalkyl, C1-4-alkoxy, hydroxy-C1-4-alkoxy, —NH₂, methylamino, dimethylamino, 6 membered heterocyclic ring, sulfamoyl, dimethylsulfamoyl, methylsulfonyl, methylsulfonyloxo, cyano, —(C═O)R′, a halogen-phenyl group, and a monocyclic or bicyclic heterocyclic group, wherein R′ is as defined above;

and
X₄ and X₅ independently of each other represent
hydrogen, C1-4-alkyl, halogen-C1-4-alkyl, C3-6-cycloalkyl, halogen, nitro, —NH₂, methoxycarbonyl, acetyl, methoxycarbamoyl or cyano.

In some embodiments of any of the methods, the antagonist of KDM5 is concomitantly administered with the cancer therapy agent (e.g., targeted therapy, chemotherapy, and/or radiotherapy). In some embodiments, the antagonist of KDM5 is administered prior to and/or concurrently with the cancer therapy agent (e.g., targeted therapy, chemotherapy, and/or radiotherapy).

In some embodiments of any of the methods, the cancer is lung cancer, breast cancer, pancreatic cancer, colorectal cancer, and/or melanoma. In some embodiments, the cancer is lung. In some embodiments, the lung cancer is NSCLC. In some embodiments, the cancer is breast cancer. In some embodiments, the cancer is melanoma.

### BRIEF DESCRIPTION OF THE FIGURES

FIG. 1|(A) Schematic of histone 3 (H3) tail and amino acid positions of post-translational modification. KDM5 is a demethylase capable of removing tri- and di-methyl marks from lysine 4 of H3. (B) KDM5 is also known as JARID1. The KDM5/JARID1 family of demethylases in humans contains four members, KDM5A, KDM5B, KDM5C, and KDM5D. As shown in the schematic, KDM5 family members contain five conserved domains: JmjN, ARID, JmjC, PHD and a C₅HC₂ zinc finger.

FIG. 2|(A) Both KDM5A and KDM5B are upregulated in the human non-small-cell-lung cancer line PC9 drug tolerant persisters (DTPs) compared to parental PC9 cells. (B) Relative expression levels of KDM5A mRNA is enriched in neoadjuvant lung adenocarcinoma patient samples compared to naïve lung adenocarcinoma patients. (C) H3K4 me³ and H3K4 me² are reduced in PC9 DTP compared to PC9 parental cells as shown by Western blot. (D) H3K4 me³ is reduced in PC9 DTP compared to PC9 parental cells as shown as shown by MSD ELISA.

FIG. 3|(A) Schematic of KDM5A demethylase-catalytic dead mutant. (H483A based on the numbering of SEQ ID NO:1.) (B) Expression of KDM5A shorthairpin with 3′-UTR-GFP knockdown eliminates PC9 drug tolerant cells (data not shown). The elimination of PC9 drug tolerant cells by KDM5A shorthairpin with 3′-UTR-GFP knockdown can be rescued by co-expression of KDM5A wild-type-FLAG tagged. (C) A KDM5A demethylase-catalytically inactive mutant, however, is unable to rescue elimination of PC9 drug tolerant cells by KDM5A shorthairpin with 3′-UTR-GFP knockdown. This suggests that KDM5A demethylase activity is required for the establishment of drug-tolerance. In the experiment, drug-tolerant cells lose knockdown of endogenous gene, unless wt KDM5a is present.

FIG. 4|(A) Table of KDM5 tool compounds and relative KDM5A IC50, KDM2/3 IC50, H3K4 me³ EC50, the compounds' cellular permeability as measured in MDCK cells (A:B, apical-to-basolateral), and the compounds' measured human plasma protein binding. (B) Western blot of PC9 cells incubated with CPI-550 or CPI-766. CPI-766 inhibits demethylation of H3K4 and an accumulation of H3K4 me³ is observed. (C) Graphical representation of H3K4 me³/H3 at various concentration s of CPI-550 and CPI-766 as measured by MSD ELISA.

FIG. 5|Comparison of H3 K4 marks on PC9 cells treated with KDM5A inhibitor CPI-766 or inactive control CPI-550 (inactive) by mass spectrometry. (A) Mole fraction (relative abundances) of H3K4 unmodified, monomethylated, dimethylated, trimethylated, and acetylated in CPI-550 and CPI-766 treated cells. (B) Log 2 ratios of peak areas of H3K4 unmodified, monomethylated, dimethylated, trimethylated, and acetylated in CPI-766 treatment to CPI-550 treatment (0 means no change, +1 is two fold increase etc.).

FIG. 6|Antagonists of KDM5, CPI-455 and PCI-766, increase H3K4me³ in multiple tested models (A) PC9, (B) SKBR3, (C) H441, and (D) H596) by MSD ELISA.

FIG. 7|Active KDM5i alone do not substantially affect cell number as measured after 96 hours in drug at concentrations below 50 uM in PC9 cells (A) and concentrations below 25 uM in SKBR3 (B). However, no substantial differences at these concentrations could be seen even at 30 days in drug (data not shown).

FIG. 8|Binding small molecule KDM5 inhibitors disrupt drug tolerance. (A1-2) PC9 cells were incubated with 25 uM of active KDM5 compounds or inactive controls for 5 days prior to plating the cells in 1uM Tarceva. Plates were stained 30 days following Tarceva treatment. Similar experiments were also done in several other models. For example, SKBR3 (B1-3 and C1-2), HCC1954, H441, with various drugs. In all cases the KDM5 inhibitor has no effect on the proliferation or survival of the parental population.

FIG. 9|Effect of siRNA knockdown of KDM5A in H1299 treated with the taxane, paclitaxel, using different specific siRNAs (Dharmacon siGenome (×4)). Z scores in the media and paclitaxel conditions are presented on the X and Y axis, respectively. KDM5A knockdown data are presented along with the data for other chromatin modifier genes (Epi300 library siRNA), the non-targeting (NTC) and siTOX controls in similar treatment conditions.

FIG. 10|Modulation of KDM5 and H3K4Me3 levels in H441 DTPs. (A-B) Western blots showing decreased H3K4me3 (A) and increased KDM5A and KDM5B (B) levels in chemotherapy-treated H441 cells. (C-D) H441 cells were plated with 25 uM of active or inactive KDM5 compounds for 3 days prior to treatment with 5 cycles of carboplatin (5.38 μM)+paclitaxel (1.25 μM). Treatment with active KDM5 compound disrupts DTPs.

FIG. 11|(A-B) Pretreatment with CPI-766 KDM5 inhibitor for 5 days reduced the number of irradiation tolerant PC9 cells. (C) Pretreatment of PC9 cells for 5 days with active KDM5 inhibitors CPI-445 and CPI-766 decreases the number of cells following γ-irradiation compared to inactive controls.

FIG. 12|Identifying the melanoma cancer cell model for DTP development. (A) Drug dose response experiment to determine GI50 for vemurafenib in the chosen Colo-829 cells. Cell viability assay was performed using cell titer Glo readout after 4 days of incubation with 8 different doses of vemurafenib. (B) Photomicrograph showing Colo-829 control cells (i) as compared to DTPs after 11 days of treatment with vemurafenib (ii).

FIG. 13|Assay development to perform DTP assay in colo-829 melanoma cell line in a semi high throughput format. (A) Photomicrographs acquired on incucyte zoom from cells that constitutively express a red-fluorescent marker in the nucleus (Nuc-Red). Due to the presence of the nuclear marker, the data was acquired in real time throughout the course of the experiment. Shown are positive control cells (i, iii and v) and DTPs (ii, iv and vi) in 6, 12 and 24 well format, respectively. (B) Line graphs showing the establishment of DTPs in 20 μM vemurafenib-treated Colo-829 cells in 6, 12 and 24 well format, respectively. (C) Bar graphs showing the number of Nuc-Red positive cells in the DMSO and inhibitor treated wells upon completion of the experiment. (D) Bar graph comparing the number of DTP's obtained in the 6, 12 and 24 well assay plates. 6 and 12 well formats look very comparable, so the 12 well plates were selected.

FIG. 14|KDM5 inhibitors suppress DTP formation. (A) Graphs showing the raw data across the entire span of the experiment looking at the differences in DTP formation when Colo-829 cells are pre-treated with 25 μM of KDM5 active (CPI-766) or inactive (CPI-550) inhibitors 5 days prior to the addition of vemurafenib. The data was acquired real time throughout the course of the experiment (B) Histogram plot of the above mentioned data depicting robust reduction in the number of DTPs formed when cells are pre-treated with CPI-766 as compared to CPI-550 or the DMSO controls. (C) Histogram showing the reduction in the number of DTP's formed upon treatment with CPI-766 relative to CPI-550 and the DMSO control.

FIG. 15|DTP assay in the presence of different doses of CPI-766 and CPI-550 to determine if there is a dose dependent reduction in DTPs. (A) Histogram showing dose dependent reduction in the number of DTPs formed after pre-treatment with varying doses of CPI-766 and CPI-550.

FIG. 16|Binding small molecule KDM5 inhibitors disrupt drug tolerance. nuc-RED PC9 cells were incubated with various concentrations of active KDM5 compound CPI-382 (B) or inactive control CPI-383 (A) for 5 days prior to plating the cells in 1 uM Tarceva. Plates were stained 30 days following Tarceva treatment.

FIG. 17|FIG. 17 (A and B) provides results illustrating that a KDM5 inhibitor blocks drug tolerance of a colorectal cancer cell line.

### DETAILED DESCRIPTION

### I. Definitions

An “antagonist” (interchangeably termed “inhibitor”) of a polypeptide of interest is an agent that interferes with activation or function of the polypeptide of interest, e.g., partially or fully blocks, inhibits, or neutralizes a biological activity mediated by a polypeptide of interest. For example, an antagonist of polypeptide X may refers to any molecule that partially or fully blocks, inhibits, or neutralizes a biological activity mediated by polypeptide X. Examples of inhibitors include antibodies; ligand antibodies; small molecule antagonists; antisense and inhibitory RNA (e.g., shRNA) molecules. Preferably, the inhibitor is an antibody or small molecule which binds to the polypeptide of interest. In a particular embodiment, an inhibitor has a binding affinity (dissociation constant) to the polypeptide of interest of about 1,000 nM or less. In another embodiment, inhibitor has a binding affinity to the polypeptide of interest of about 100 nM or less. In another embodiment, an inhibitor has a binding affinity to the polypeptide of interest of about 50 nM or less. In a particular embodiment, an inhibitor is covalently bound to the polypeptide of interest. In a particular embodiment, an inhibitor inhibits signaling of the polypeptide of interest with an IC₅₀ of 1,000 nM or less. In another embodiment, an inhibitor inhibits signaling of the polypeptide of interest with an IC₅₀ of 500 nM or less. In another embodiment, an inhibitor inhibits signaling of the polypeptide of interest with an IC₅₀ of 50 nM or less. In certain embodiments, the antagonist reduces or inhibits, by at least 10%, 20%, 30%, 40%, 50%, 60%, 70%, 80%, 90% or more, the expression level or biological activity of the polypeptide of interest. In some embodiments, the polypeptide of interest is KDM5.

The term “polypeptide” as used herein, refers to any native polypeptide of interest from any vertebrate source, including mammals such as primates (e.g., humans) and rodents (e.g., mice and rats), unless otherwise indicated. The term encompasses “full-length,” unprocessed polypeptide as well as any form of the polypeptide that results from processing in the cell. The term also encompasses naturally occurring variants of the polypeptide, e.g., splice variants or allelic variants.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “small molecule” refers to any molecule with a molecular weight of about 2000 daltons or less, preferably of about 500 daltons or less.

Examples of small molecule antagonists of KDM5 that may be useful in the practice of certain embodiments include compounds of Formula I or II, an isomer or a mixture of isomers thereof or a pharmaceutically acceptable salt, solvate or prodrug thereof. Such compounds, and processes and intermediates that are useful for preparing such compounds, are described in WO 2012/007007 and WO 2012/007008.

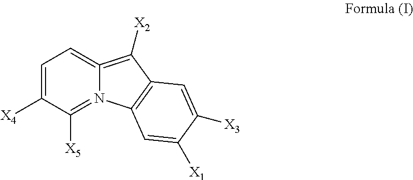

Wherein X₁ represents -A-B, wherein
A represents a bond, O, S or NH, and
B represents
C1-6-alkyl, C2-4-alkenyl or C2-4-alkynyl,

which C1-6-alkyl, C2-4-alkenyl or C2-4-alkynyl may optionally be substituted with one or more substituents selected from the group consisting of hydroxy, C3-6-cycloalkyl, C1-4-alkoxy, hydroxy-C1-4-alkoxy, halo, trifluoromethyl, —NH₂, methylamino, dimethylamino, sulfamoyl, dimethylsulfamoyl, methylsulfonyl, methylsulfonyloxo, methylsulfinyl, methylsulfanyl, cyano, —(C═O)R′, a phenyl group, and a monocyclic or bicyclic heterocyclic group,

where

R′ represents hydroxy, C1-4-alkyl, halo-C1-4-alkyl, C1-4-alkoxy, —NH₂, methylamino, dimethylamino, a phenyl group or a monocyclic or bicyclic heterocyclic group;

and where

the phenyl group may be substituted with one or more of the substituents selected from the group consisting of methyl, trifluoromethyl, halo, cyano, acetamino, methylsulfonylamino, and a monocyclic or bicyclic heterocyclic group;

### —OH or —(C═O)R″,

where R″ represents hydrogen, hydroxy, C1-4-alkyl, cyclopropyl, halo-C1-4-alkyl, C1-4-alkoxy, —COOH, —NH₂, methylamino, dimethylamino, methylsulfonyl, or a monocyclic or bicyclic heterocyclic group; or

where R″ represents C1-4-alkyl, C1-4-alkoxy, oxy, carbamoyl, amine or a monocyclic or bicyclic heterocyclic group, which is substituted with one or more substituents selected from the group consisting of hydroxy, methyl, ethyl, —O—C1-6-alkyl, hydroxymethyl, hydroxymethyl, methoxyethyl, acetyl, cyano, ethoxycarbonyl, dimethylamino, N-[3(dimethylamino)propyl]N′ethylcarbamimidoyl, methylsulfinyl, methylsulfanyl, methylsulfonyl, methoxyethoxyethyl, (dimethylamino)ethyl and methylsulfanylethyl, which —O—C1-6-alkyl may optionally be substituted with hydroxy, methoxy or dimethylamino;

### —(C═S)R′″,

where R′″ represents —NH₂, methylamino or dimethylamino;

### —C(CH3)=N—R″,

where R″″ represents hydroxy or methoxy;

sulfamoyl, dimethylsulfamoyl, sulfinyl or sulfonyl,

which sulfamoyl, sulfonyl or sulfonyl may optionally be substituted with one or more substituents selected from the group consisting of C1-4-alkyl, halo-C1-4-alkyl, methoxy-C1-4-alkyl, dimethylamino, (dimethylamino)methyl, (dimethylamino)ethyl, C3-6-cycloalkyl, C2-4-alkenyl and a monocyclic or bicyclic heterocyclic group;

fluoro, chloro, bromo or cyano; or
a monocyclic or bicyclic heterocyclic group,
where the monocyclic or bicyclic heterocyclic group may optionally be substituted with one or more substituents selected from the group consisting of C1-2-alkyl, halo, halo-C1-2-alkyl, C1-4-alkoxy, C1-4-alkoxycarbonyl, COOH, cyano, —NH₂, methylamino and dimethylamino;
and
X₂ represents
C1-18-alkyl, C2-18-alkenyl, or C2-18-alkynyl,

which C1-18-alkyl, C2-18-alkenyl, C2-18-alkynyl may optionally be substituted with one or more substituents selected from the group consisting of C3-6-cycloalkyl, hydroxy, halo, trifluoromethyl, C1-6-alkoxy, hydroxy-C1-6-alkoxy, C1-6-alkyl-C1-6-alkoxy, trifluoromethyi-C1-6-alkoxy, oxo-C1-6-alkyl, —NH₂, dimethylamino, cyano, phenyl, a 5-membered monocyclic heterocyclic group, or a 6-membered monocyclic heterocyclic group, which phenyl, 5-membered monocyclic heterocyclic group, or 6-membered monocyclic heterocyclic group may optionally be substituted with one or more substituents selected from the group consisting of C1-6-alkyl or halo; or

—O-Xa, —(C═O)—O-Xb, —(C═O)-Xc, —NXd1Xd2, —(CO)—NXE1XE2, or —(C═O)—NH—SO₂-Xf, where Xa, Xb, Xc, Xd1, Xd2, Xe1, Xe2 or Xf independently of each other represent hydrogen, C1-18-alkyl, C2-18-alkenyl, C2-18-alkynyl, C3-6-cycloalkyl, phenyl, a 5-membered monocyclic heterocyclic group or a 6-membered monocyclic heterocyclic group;

which C1-18-alkyl, C2-18-alkenyl, C2-18-alkynyl, C3-6-cycloalkyl, phenyl, a 5-membered monocyclic heterocyclic group or a 6-membered monocyclic heterocyclic group may optionally be substituted with one or more substituents selected from the group consisting of C3-6-cycloalkyl, hydroxy, halo, trifluoromethyl, C1-4-alkyl, C1-6-alkoxy, C1-6-alkoxycarbonyl, C1-4-alkylamino, hydroxy-C1-6-alkoxy, C1-6-alkyl-C1-6-alkoxy, trifluoro-C1-6-alkoxy, trifluoromethyl-O—C1-6-alkyl, oxo-C1-6-alkyl, —NH₂, methylamino, dimethylamino, (methoxyethyl)(methyl)amino, [(dimethylamino)ethyl](methyl)amino, cyano, —O—C1-6-alkyl-phenyl, phenyl, a 5-membered monocyclic heterocyclic group, or a 6-membered monocyclic heterocyclic group, which phenyl, 5-membered monocyclic heterocyclic group, or 6-membered monocyclic heterocyclic group may optionally be substituted with one or more substituents selected from the group consisting of C1-6-alkyl, —(C═O)—O—C1-6-alkyl or halo; or

where Xe1 and Xe2 independently of each other represent hydrogen, hydroxy, C1-18-alkyl, C2-18-alkenyl, C2-18-alkynyl, C3-6-cycloalkyl, —O—C1-6-alkyl, phenyl, a 5-membered monocyclic heterocyclic group or a 6-membered monocyclic heterocyclic group,

which —O—C1-6-alkyl may optionally be substituted with hydroxy, methoxy, or dimethylamino;

with the proviso in certain embodiments that Xe1 and Xe2 cannot both represent hydrogen; and
with the proviso in certain embodiments that Xb cannot represent hydrogen; or

### —(C═O)—O—CH2-CH2-NXj1Xj2, —S-Xk, —(C═S)—N(CH3)-Xm or —(C═S)—N-Xn,

where Xj1, Xj2, Xk, Xm, Xn independently of each other represent methyl, ethyl, propyl, amino, methylamino or dimethylamino,

which methyl, ethyl or propyl may optionally be substituted with one or more substituents selected from the group consisting of methoxycarbonyl, dimethylamino, carbamoyl, phenyl, cyanophenyl, and a 5- or 6-membered monocyclic heterocyclic group; and

X₃ represents hydrogen, C1-4-alkyl, C2-4-alkenyl, C2-4-alkynyl or
—O-Xg —S-Xh or —NXi1Xi2 where X9g, Xh, Xi1 and Xi2 independently of each other represent hydrogen, —(CH2)n-CH3, or —(CH2)n-COOH, where n is 0, 1, 2, 3 or 4
and
X₄ and X₅ independently of each other represent
hydrogen, C1-4-alkyl, halo-C1-4-alkyl, C3-6-cycloalkyl, halo, nitro, —NH₂, or cyano.

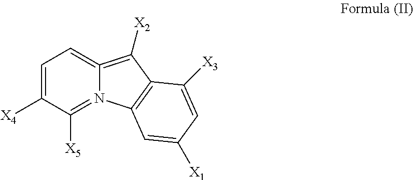

### Wherein

X₁ represents -A-B, wherein
A represents a bond, O, S, or NH, and
B represents
C1-6-alkyl, C2-4-alkenyl, C2-4-alkynyl or C3-5-cycloalkyl which C1-6-alkyl, C2-4-alkenyl, C2-4-alkynyl or C3-5-cycloalkyl may optionally be substituted with one or more substituents selected from the group consisting of hydroxy, C3-6-cycloalkyl, C1-4-alkoxy, hydroxy-C1-4-alkoxy, —NH₂, methylamino, dimethylamino, sulfamoyl, dimethylsulfamoyl, methylsulfonyl, methylsulfonyloxo, cyano, —(C═O)R′, a phenyl group, and a monocyclic or bicyclic heterocyclic group,

where

R′ represents hydroxy, C1-4-alkyl, halogen-C1-4-alkyl, C1-4-alkoxy, —NH₂, methylamino, cyclopropyl, dimethylamino, a phenyl group or a monocyclic or bicyclic heterocyclic group;

and where

the phenyl group may be substituted with one or more of the substituents selected from the group consisting of methyl, trifluoromethyl, halogen, cyano, acetamino, methylsulfonylamino, and a monocyclic or bicyclic heterocyclic group; or

—OH, with the proviso in certain embodiments that B only represent —OH, when A is a bond; or

### or —(C═O)R″,

where R″ represents hydroxy, halogen-C1-4-alkyl, C1-4-alkoxy, hydroxy-C1-4-alkoxy, —NH₂, C1-3-alkyl-amino, di-C1-3-alkyl-amino, methylsulfonyl, a monocyclic or bicyclic heterocyclic group, C3-4-cycloalkyl or C1-4-alkyl, wherein said C3-4-cycloalkyl or C1-4 alkyl optionally may be substituted with one or more substituents selected from the group consisting of hydroxy, C3-6-cycloalkyl, C1-3-alkoxy, hydroxy-C1-3-alkoxy, —NH₂, methylamino, dimethylamino, 6 membered heterocyclic ring, sulfamoyl, dimethylsulfamoyl, methylsulfonyl, methylsulfonyloxo, cyano, —(C═O)R′, a halo-phenyl group, and a monocyclic or bicyclic heterocyclic group, where R′ is as identified above;

### —(C═O)NH—R′″,

where R′″ represents hydroxyethyl, methoxyethyl, dimethylaminoethyl, methanesulfonyl or —O—C1-6-alkyl optionally substituted with dimethylamino;

sulfamoyl, sulfinyl, sulfanyl or sulfonyl,

which sulfamoyl may optionally be substituted with one or two C1-3-alkyl groups and said sulfinyl, sulfanyl or sulfonyl may optionally be substituted with one substituent selected from the group consisting of C1-4-alkyl, halogen-C1-4-alkyl, carbonyi-C1-3-alkyl, methylsulfamoyl, C3-6-cycloalkyl, C1-3-alkyl-amino, di-C1-3-alkyl-amino, dimethylaminoethyl, a 6 membered heterocyclic ring, and a monocyclic or bicyclic heterocyclic group;

a phenyl, monocyclic or bicyclic heterocyclic group,

where the phenyl, monocyclic or bicyclic heterocyclic group may optionally be substituted with one or more substituents selected from the group consisting of halogen, halogen-C1-3-alkyl, C1-3-alkoxy, C1-3-alkoxyalkoxy, C1-3-alkoxycarbonyl, COOH, cyano, —NH₂, methylamino, dimethylamino, cyclopropyl and C1-3-alkyl, wherein said cyclopropyl or C1-3 alkyl optionally may be substituted with one or more substituents selected from the group consisting of hydroxy, cyclopropyl, C1-3-alkoxy, hydroxy-C1-3-alkoxy, —NH₂, methylamino, dimethylamino, 6 membered heterocyclic ring, sulfamoyl, dimethylsulfamoyl, methylsulfonyl, methylsulfonyloxo, cyano, —(C═O)R′, a halogen-phenyl group, and a monocyclic or bicyclic heterocyclic group, where R′ is as identified above;

and
X₂ represents

—COOH, (C═O)NH₂ or —CN

and
X₃ represents

Hydrogen or —OH; or

—Y-Xa-Xb

where

Y is O, C═O or a bond; and

Xa is -a bond, C1-18-alkyl, C2-18-alkenyl, C2-18-alkynyl, C3-10-cycloalkyl, —C1-18-alkylO-, —O— or —NXb-, with the proviso in certain embodiments that when Y is O then Xa is not 0; and each Xb is individually —H, C3-6-cycloalkyl, C1-6 alkoxy, phenyl, phenoxy, a 5-membered monocyclic heterocyclic group, a 6-membered monocyclic heterocyclic group or a bicyclic heteroaromatic group, which C3-10-cycloalkyl, C1-6 alkoxy, phenyl, phenoxy, 5-membered monocyclic heterocyclic group, 6-membered monocyclic heterocyclic group or bicyclic heteroaromatic group may optionally be substituted with one or more substituents selected from the group consisting of halogen, halogen-C1-4-alkyl, hydroxy linear or branched C1-4-alkoxy, C1-6-alkoxyalkoxy, C1-4-alkoxycarbonyl, C1-4-alkylcarbonyl, COOH, cyano, —NH₂, methylamino, dimethylamino, hydroxy and linear or branched C1-5-alkyl, wherein said C1-5 alkyl optionally may be substituted with one or more substituents selected from the group consisting of hydroxy, C3-6-cycloalkyl, C1-4-alkoxy, hydroxy-C1-4-alkoxy, —NH₂, methylamino, dimethylamino, 6 membered heterocyclic ring, sulfamoyl, dimethylsulfamoyl, methylsulfonyl, methylsulfonyloxo, cyano, —(C═O)R′, a halogen-phenyl group, and a monocyclic or bicyclic heterocyclic group, wherein R′ is as defined above;

and
X₄ and X₅ independently of each other represent
hydrogen, C1-4-alkyl, halogen-C1-4-alkyl, C3-6-cycloalkyl, halogen, nitro, —NH₂, methoxycarbonyl, acetyl, methoxycarbamoyl or cyano.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The terms anti-polypeptide of interest antibody and “an antibody that binds to” a polypeptide of interest refer to an antibody that is capable of binding a polypeptide of interest with sufficient affinity such that the antibody is useful as a diagnostic and/or therapeutic agent in targeting a polypeptide of interest. In one embodiment, the extent of binding of an anti-polypeptide of interest antibody to an unrelated, non-polypeptide of interest protein is less than about 10% of the binding of the antibody to a polypeptide of interest as measured, e.g., by a radioimmunoassay (RIA). In certain embodiments, an antibody that binds to a polypeptide of interest has a dissociation constant (Kd) of ≦1 μM, ≦100 nM, ≦10 nM, ≦1 nM, ≦0.1 nM, ≦0.01 nM, or ≦0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-polypeptide of interest antibody binds to an epitope of a polypeptide of interest that is conserved among polypeptides of interest from different species. In some embodiments, the polypeptide of interest is KDM5.

A “blocking antibody” or an “antagonist antibody” is one which inhibits or reduces biological activity of the antigen it binds. Preferred blocking antibodies or antagonist antibodies substantially or completely inhibit the biological activity of the antigen.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

The term “chimeric” antibody refers to an antibody in which a portion of the heavy and/or light chain is derived from a particular source or species, while the remainder of the heavy and/or light chain is derived from a different source or species.

The terms “full length antibody,” “intact antibody,” and “whole antibody” are used herein interchangeably to refer to an antibody having a structure substantially similar to a native antibody structure or having heavy chains that contain an Fc region.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

As used herein, the term “targeted therapeutic” refers to a therapeutic agent that binds to polypeptide(s) of interest and inhibits the activity and/or activation of the specific polypeptide(s) of interest. Examples of such agents include antibodies and small molecules that bind to the polypeptide of interest.

A “chemotherapy” refers to a chemical compound useful in the treatment of cancer. Examples of chemotherapies include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

The term “cytotoxic agent” as used herein refers to a substance that inhibits or prevents a cellular function and/or causes cell death or destruction. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu), chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents), growth inhibitory agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

An “immunoconjugate” is an antibody conjugated to one or more heterologous molecule(s), including but not limited to a cytotoxic agent.

“Individual response” or “response” can be assessed using any endpoint indicating a benefit to the individual, including, without limitation, (1) inhibition, to some extent, of disease progression (e.g., cancer progression), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e. reduction, slowing down or complete stopping) of metastasis; (5) relief, to some extent, of one or more symptoms associated with the disease or disorder (e.g., cancer); (6) increase in the length of progression free survival; and/or (7) decreased mortality at a given point of time following treatment.

The term “substantially the same,” as used herein, denotes a sufficiently high degree of similarity between two numeric values, such that one of skill in the art would consider the difference between the two values to be of little or no biological and/or statistical significance within the context of the biological characteristic measured by said values (e.g., Kd values or expression). The difference between said two values is, for example, less than about 50%, less than about 40%, less than about 30%, less than about 20%, and/or less than about 10% as a function of the reference/comparator value.

The phrase “substantially different,” as used herein, denotes a sufficiently high degree of difference between two numeric values such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

An “effective amount” of a substance/molecule, e.g., pharmaceutical composition, refers to an amount effective, at dosages and for periods of time necessary, to achieve the desired therapeutic or prophylactic result.

A “therapeutically effective amount” of a substance/molecule may vary according to factors such as the disease state, age, sex, and weight of the individual, and the ability of the substance/molecule to elicit a desired response in the individual. A therapeutically effective amount is also one in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The phrase “pharmaceutically acceptable salt” as used herein, refers to pharmaceutically acceptable organic or inorganic salts of a compound.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “concomitantly” is used herein to refer to administration of two or more therapeutic agents, give in close enough temporal proximity where their individual therapeutic effects overlap in time. Accordingly, concurrent administration includes a dosing regimen when the administration of one or more agent(s) continues after discontinuing the administration of one or more other agent(s). In some embodiments, the concomitantly administration is concurrently, sequentially, and/or simultaneously.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In certain embodiments, the manufacture or kit is promoted, distributed, or sold as a unit for performing the methods described herein.

As is understood by one skilled in the art, reference to “about” a value or parameter herein includes (and describes) embodiments that are directed to that value or parameter per se. For example, description referring to “about X” includes description of “X”.

It is understood that aspect and embodiments of the invention described herein include “consisting” and/or “consisting essentially of” aspects and embodiments. As used herein, the singular form “a”, “an”, and “the” includes plural references unless indicated otherwise.

### II. Methods and Uses

Provided herein are methods of using antagonist of KDM5, for example, for treating cancer and/or preventing drug resistance (e.g., in single agent and/or combination therapy). For example, a method of treating cancer in an individual comprising administering to the individual an antagonist of KDM5 alone or in combination with a cancer therapy agent. In some embodiments, the individual is selected for treatment with a cancer therapy agent (e.g., targeted therapies, chemotherapies, and/or radiotherapies). In some embodiments, the individual starts treatment comprising administration of the antagonist of KDM5 prior to treatment with the cancer therapy agent. In some embodiments, the individual concurrently receives treatment comprising the antagonist of KDM5 and the cancer therapy agent. In some embodiments, the antagonist of KDM5 increases the period of cancer sensitivity and/or delays development of cancer resistance. In some embodiments, the antagonist of KDM5 is an antagonist of one or more of KDM5A, KMD5B, KDM5C, and/or KDM5D. In some embodiments, the antagonist of KDM5 is an antagonist of KDM5A and/or KDM5B.

Also provided herein are methods of utilizing an antagonist of KDM5 and a cancer therapy agent (e.g., targeted therapy, chemotherapy, and/or radiotherapy).

In particular, provided herein are methods of treating cancer in an individual comprising administering to the individual (a) an antagonist of KDM5 and (b) a cancer therapy agent (e.g., targeted therapy, chemotherapy, and/or radiotherapy). In some embodiments, the respective amounts of the antagonist of KDM5 and the cancer therapy agent are effective to increase the period of cancer sensitivity and/or delay the development of cell resistance to the cancer therapy agent. In some embodiments, the respective amounts of the antagonist of KDM5 and the cancer therapy agent are effective to increase efficacy of a cancer treatment comprising the cancer therapy agent. For example, in some embodiments, the respective amounts of the antagonist of KDM5 and the cancer therapy agent are effective to increase efficacy compared to a treatment (e.g., standard of care treatment) comprising administering an effective amount of cancer therapy agent without (in the absence of) the antagonist of KDM5. In some embodiments, the respective amounts of the antagonist of KDM5 and the cancer therapy agent are effective to increase response (e.g., complete response) compared to a treatment (e.g., standard of care treatment) comprising administering an effective amount of cancer therapy agent without (in the absence of) the antagonist of KDM5. In some embodiments, the antagonist of KDM5 and the cancer therapy agent are administered concomitantly. In some embodiments, the cancer therapy agent is a targeted therapy, chemotherapy, and/or radiotherapy. In some embodiments, the targeted therapy and/or chemotherapy is one or more of an EGFR antagonist, RAF inhibitor, PI3K inhibitor, taxane, and platinum agent. In some embodiments, the combination therapy comprises (a) an antagonist of KDM5 and (b) EGFR antagonist. In some embodiments, the combination therapy comprises (a) an antagonist of KDM5 and (b) RAF inhibitor. In some embodiments, the combination therapy comprises (a) an antagonist of KDM5 and (b) PI3K inhibitor. In some embodiments, the combination therapy comprises (a) an antagonist of KDM5 and (b) taxane (e.g., paclitaxel). In some embodiments, the combination therapy comprises (a) an antagonist of KDM5 and (b) platinum agent (e.g., carboplatin or cisplatin). In some embodiments, the combination therapy comprises (a) an antagonist of KDM5, (b) taxane (e.g., paclitaxel), and (c) platinum agent (e.g., carboplatin or cisplatin). In some embodiments, the taxane is paclitaxel. In some embodiments, the antagonist of KDM5 is an antagonist of one or more of KDM5A, KMD5B, KDM5C, and/or KDM5D. In some embodiments, the antagonist of KDM5 is an antagonist of KDM5A and/or KDM5B.

Further provided herein are methods of increasing efficacy of a cancer treatment comprising a cancer therapy agent (e.g., targeted therapy, chemotherapy, and/or radiotherapy) in an individual comprising administering to the individual (a) an effective amount of an antagonist of KDM5 and (b) an effective amount of the cancer therapy agent. In some embodiments, the antagonist of KDM5 and the cancer therapy agent are administered concomitantly. In some embodiments, the cancer therapy agent is a targeted therapy, chemotherapy, and/or radiotherapy. In some embodiments, the targeted therapy and/or chemotherapy is one or more of an EGFR antagonist, RAF inhibitor, PI3K inhibitor, taxane, and platinum agent. In some embodiments, the combination therapy comprises (a) an antagonist of KDM5 and (b) EGFR antagonist. In some embodiments, the combination therapy comprises (a) an antagonist of KDM5 and (b) RAF inhibitor. In some embodiments, the combination therapy comprises (a) an antagonist of KDM5 and (b) PI3K inhibitor. In some embodiments, the combination therapy comprises (a) an antagonist of KDM5 and (b) taxane (e.g., paclitaxel). In some embodiments, the combination therapy comprises (a) an antagonist of KDM5 and (b) platinum agent (e.g., carboplatin or cisplatin). In some embodiments, the combination therapy comprises (a) an antagonist of KDM5, (b) taxane (e.g., paclitaxel), and (c) platinum agent (e.g., carboplatin or cisplatin). In some embodiments, the taxane is paclitaxel. In some embodiments, the antagonist of KDM5 is an antagonist of one or more of KDM5A, KMD5B, KDM5C, and/or KDM5D. In some embodiments, the antagonist of KDM5 is an antagonist of KDM5A and/or KDM5B.

Provided herein methods of treating cancer in an individual wherein cancer treatment comprising administering to the individual (a) an effective amount of an antagonist of KDM5 and (b) an effective amount of a cancer therapy agent (e.g., targeted therapy, chemotherapy, and/or radiotherapy), wherein the cancer treatment has increased efficacy compared to a treatment (e.g., standard of care treatment) comprising administering an effective amount of cancer therapy agent without (in the absence of) the antagonist of KDM5. In some embodiments, the antagonist of KDM5 and the cancer therapy agent are administered concomitantly. In some embodiments, the cancer therapy agent is a targeted therapy, chemotherapy, and/or radiotherapy. In some embodiments, the targeted therapy and/or chemotherapy is one or more of an EGFR antagonist, RAF inhibitor, PI3K inhibitor, taxane, and platinum agent. In some embodiments, the combination therapy comprises (a) an antagonist of KDM5 and (b) EGFR antagonist. In some embodiments, the combination therapy comprises (a) an antagonist of KDM5 and (b) RAF inhibitor. In some embodiments, the combination therapy comprises (a) an antagonist of KDM5 and (b) PI3K inhibitor. In some embodiments, the combination therapy comprises (a) an antagonist of KDM5 and (b) taxane (e.g., paclitaxel). In some embodiments, the combination therapy comprises (a) an antagonist of KDM5 and (b) platinum agent (e.g., carboplatin or cisplatin). In some embodiments, the combination therapy comprises (a) an antagonist of KDM5, (b) taxane (e.g., paclitaxel), and (c) platinum agent (e.g., carboplatin or cisplatin). In some embodiments, the taxane is paclitaxel. In some embodiments, the antagonist of KDM5 is an antagonist of one or more of KDM5A, KMD5B, KDM5C, and/or KDM5D. In some embodiments, the antagonist of KDM5 is an antagonist of KDM5A and/or KDM5B.

In addition, provided herein are methods of delaying and/or preventing development of cancer resistant to a cancer therapy agent (e.g., targeted therapy, chemotherapy, and/or radiotherapy) in an individual, comprising administering to the individual (a) an effective amount of an antagonist of KDM5 and (b) an effective amount of the cancer therapy agent. In some embodiments, the antagonist of KDM5 and the cancer therapy agent are administered concomitantly. In some embodiments, the cancer therapy agent is a targeted therapy, chemotherapy, and/or radiotherapy. In some embodiments, the targeted therapy and/or chemotherapy is one or more of an EGFR antagonist, RAF inhibitor, PI3K inhibitor, taxane, and platinum agent. In some embodiments, the combination therapy comprises (a) an antagonist of KDM5 and (b) EGFR antagonist. In some embodiments, the combination therapy comprises (a) an antagonist of KDM5 and (b) RAF inhibitor. In some embodiments, the combination therapy comprises (a) an antagonist of KDM5 and (b) PI3K inhibitor. In some embodiments, the combination therapy comprises (a) an antagonist of KDM5 and (b) taxane (e.g., paclitaxel). In some embodiments, the combination therapy comprises (a) an antagonist of KDM5 and (b) platinum agent (e.g., carboplatin or cisplatin). In some embodiments, the combination therapy comprises (a) an antagonist of KDM5, (b) taxane (e.g., paclitaxel), and (c) platinum agent (e.g., carboplatin or cisplatin). In some embodiments, the taxane is paclitaxel. In some embodiments, the antagonist of KDM5 is an antagonist of one or more of KDM5A, KMD5B, KDM5C, and/or KDM5D. In some embodiments, the antagonist of KDM5 is an antagonist of KDM5A and/or KDM5B.

Provided herein are methods of treating an individual with cancer who has increased likelihood of developing resistance to a cancer therapy agent (e.g., targeted therapy, chemotherapy, and/or radiotherapy) comprising administering to the individual (a) an effective amount of an antagonist of KDM5 and (b) an effective amount of the cancer therapy agent. In some embodiments, the antagonist of KDM5 and the cancer therapy agent are administered concomitantly. In some embodiments, the cancer therapy agent is a targeted therapy, chemotherapy, and/or radiotherapy. In some embodiments, the targeted therapy and/or chemotherapy is one or more of an EGFR antagonist, RAF inhibitor, PI3K inhibitor, taxane, and platinum agent. In some embodiments, the combination therapy comprises (a) an antagonist of KDM5 and (b) EGFR antagonist. In some embodiments, the combination therapy comprises (a) an antagonist of KDM5 and (b) RAF inhibitor. In some embodiments, the combination therapy comprises (a) an antagonist of KDM5 and (b) PI3K inhibitor. In some embodiments, the combination therapy comprises (a) an antagonist of KDM5 and (b) taxane (e.g., paclitaxel). In some embodiments, the combination therapy comprises (a) an antagonist of KDM5 and (b) platinum agent (e.g., carboplatin or cisplatin). In some embodiments, the combination therapy comprises (a) an antagonist of KDM5, (b) taxane (e.g., paclitaxel), and (c) platinum agent (e.g., carboplatin or cisplatin). In some embodiments, the taxane is paclitaxel. In some embodiments, the antagonist of KDM5 is an antagonist of one or more of KDM5A, KMD5B, KDM5C, and/or KDM5D. In some embodiments, the antagonist of KDM5 is an antagonist of KDM5A and/or KDM5B.

Further provided herein are methods of increasing sensitivity to a cancer therapy agent (e.g., targeted therapy, chemotherapy, and/or radiotherapy) in an individual with cancer comprising administering to the individual (a) an effective amount of an antagonist of KDM5 and (b) an effective amount of the cancer therapy agent. In some embodiments, the antagonist of KDM5 and the cancer therapy agent are administered concomitantly. In some embodiments, the cancer therapy agent is a targeted therapy, chemotherapy, and/or radiotherapy. In some embodiments, the targeted therapy and/or chemotherapy is one or more of an EGFR antagonist, RAF inhibitor, PI3K inhibitor, taxane, and platinum agent. In some embodiments, the combination therapy comprises (a) an antagonist of KDM5 and (b) EGFR antagonist. In some embodiments, the combination therapy comprises (a) an antagonist of KDM5 and (b) RAF inhibitor. In some embodiments, the combination therapy comprises (a) an antagonist of KDM5 and (b) PI3K inhibitor. In some embodiments, the combination therapy comprises (a) an antagonist of KDM5 and (b) taxane (e.g., paclitaxel). In some embodiments, the combination therapy comprises (a) an antagonist of KDM5 and (b) platinum agent (e.g., carboplatin or cisplatin). In some embodiments, the combination therapy comprises (a) an antagonist of KDM5, (b) taxane (e.g., paclitaxel), and (c) platinum agent (e.g., carboplatin or cisplatin). In some embodiments, the taxane is paclitaxel. In some embodiments, the antagonist of KDM5 is an antagonist of one or more of KDM5A, KMD5B, KDM5C, and/or KDM5D. In some embodiments, the antagonist of KDM5 is an antagonist of KDM5A and/or KDM5B.

In addition, provided herein are methods of extending the period of a cancer therapy agent (e.g., targeted therapy, chemotherapy, and/or radiotherapy) sensitivity in an individual with cancer comprising administering to the individual (a) an effective amount of an antagonist of KDM5 and (b) an effective amount of the cancer therapy agent. In some embodiments, the antagonist of KDM5 and the cancer therapy agent are administered concomitantly. In some embodiments, the cancer therapy agent is a targeted therapy, chemotherapy, and/or radiotherapy. In some embodiments, the targeted therapy and/or chemotherapy is one or more of an EGFR antagonist, RAF inhibitor, PI3K inhibitor, taxane, and platinum agent. In some embodiments, the combination therapy comprises (a) an antagonist of KDM5 and (b) EGFR antagonist. In some embodiments, the combination therapy comprises (a) an antagonist of KDM5 and (b) RAF inhibitor. In some embodiments, the combination therapy comprises (a) an antagonist of KDM5 and (b) PI3K inhibitor. In some embodiments, the combination therapy comprises (a) an antagonist of KDM5 and (b) taxane (e.g., paclitaxel). In some embodiments, the combination therapy comprises (a) an antagonist of KDM5 and (b) platinum agent (e.g., carboplatin or cisplatin). In some embodiments, the combination therapy comprises (a) an antagonist of KDM5, (b) taxane (e.g., paclitaxel), and (c) platinum agent (e.g., carboplatin or cisplatin). In some embodiments, the taxane is paclitaxel. In some embodiments, the antagonist of KDM5 is an antagonist of one or more of KDM5A, KMD5B, KDM5C, and/or KDM5D. In some embodiments, the antagonist of KDM5 is an antagonist of KDM5A and/or KDM5B.

Provided herein are also methods of extending the duration of response to a cancer therapy agent (e.g., targeted therapy, chemotherapy, and/or radiotherapy) in an individual with cancer comprising administering to the individual (a) an effective amount of an antagonist of KDM5 and (b) an effective amount of the cancer therapy agent. In some embodiments, the antagonist of KDM5 and the cancer therapy agent are administered concomitantly. In some embodiments, the cancer therapy agent is a targeted therapy, chemotherapy, and/or radiotherapy. In some embodiments, the targeted therapy and/or chemotherapy is one or more of an EGFR antagonist, RAF inhibitor, PI3K inhibitor, taxane, and platinum agent. In some embodiments, the combination therapy comprises (a) an antagonist of KDM5 and (b) EGFR antagonist. In some embodiments, the combination therapy comprises (a) an antagonist of KDM5 and (b) RAF inhibitor. In some embodiments, the combination therapy comprises (a) an antagonist of KDM5 and (b) PI3K inhibitor. In some embodiments, the combination therapy comprises (a) an antagonist of KDM5 and (b) taxane (e.g., paclitaxel). In some embodiments, the combination therapy comprises (a) an antagonist of KDM5 and (b) platinum agent (e.g., carboplatin or cisplatin). In some embodiments, the combination therapy comprises (a) an antagonist of KDM5, (b) taxane (e.g., paclitaxel), and (c) platinum agent (e.g., carboplatin or cisplatin). In some embodiments, the taxane is paclitaxel. In some embodiments, the antagonist of KDM5 is an antagonist of one or more of KDM5A, KMD5B, KDM5C, and/or KDM5D. In some embodiments, the antagonist of KDM5 is an antagonist of KDM5A and/or KDM5B.

In addition to providing improved treatment for cancer, administration of certain combinations described herein may improve the quality of life for a patient compared to the quality of life experienced by the same patient receiving a different treatment. For example, administration of a combination of the antagonist of KDM5 and the cancer therapy agent (e.g., targeted therapy, chemotherapy, and/or radiotherapy), as described herein to an individual may provide an improved quality of life compared to the quality of life the same patient would experience if they received only cancer therapy agent as therapy. For example, the combined therapy with the combination described herein may lower the dose of cancer therapy agent needed, thereby lessening the side-effects associated with the therapeutic (e.g. nausea, vomiting, hair loss, rash, decreased appetite, weight loss, etc.). The combination may also cause reduced tumor burden and the associated adverse events, such as pain, organ dysfunction, weight loss, etc. Accordingly, one aspect provides antagonist of KDM5 for therapeutic use for improving the quality of life of a patient treated for a cancer with a cancer therapy agent (e.g., targeted therapy, chemotherapy, and/or radiotherapy). In some embodiments, the antagonist of KDM5 and the cancer therapy agent are administered concomitantly. In some embodiments, the cancer therapy agent is a targeted therapy, chemotherapy, and/or radiotherapy. In some embodiments, the targeted therapy and/or chemotherapy is one or more of an EGFR antagonist, RAF inhibitor, PI3K inhibitor, taxane, and platinum agent. In some embodiments, the combination therapy comprises (a) an antagonist of KDM5 and (b) EGFR antagonist. In some embodiments, the combination therapy comprises (a) an antagonist of KDM5 and (b) RAF inhibitor. In some embodiments, the combination therapy comprises (a) an antagonist of KDM5 and (b) PI3K inhibitor. In some embodiments, the combination therapy comprises (a) an antagonist of KDM5 and (b) taxane (e.g., paclitaxel). In some embodiments, the combination therapy comprises (a) an antagonist of KDM5 and (b) platinum agent (e.g., carboplatin or cisplatin). In some embodiments, the combination therapy comprises (a) an antagonist of KDM5, (b) taxane (e.g., paclitaxel), and (c) platinum agent (e.g., carboplatin or cisplatin). In some embodiments, the taxane is paclitaxel. In some embodiments, the antagonist of KDM5 is an antagonist of one or more of KDM5A, KMD5B, KDM5C, and/or KDM5D. In some embodiments, the antagonist of KDM5 is an antagonist of KDM5A and/or KDM5B.

In some embodiments of any of the methods, the antagonist of KDM5 is of natural or synthetic origin. In some embodiments of any of the methods, the antagonist of KDM5 is an antibody, binding polypeptide, binding small molecule, or polynucleotide. In some embodiments, the antagonist of KDM5 binds to one or more of KDM5A, KDM5B, KDM5C, and/or KDM5D. In some embodiments, the antagonist of KDM5 binds to and/or inhibits the demethylase activity of one or more KDM5A, KDM5B, KDM5C, and/or KDM5D. In some embodiments, the KDM5 is KDM5A and/or KDM5B.

In some embodiments of any of the methods, the cancer therapy agent is a targeted therapy. In some embodiments of any of the methods, the cancer therapy agent is chemotherapy. In some embodiments of any of the methods, the cancer therapy agent is radiotherapy.

Cancer having resistance to a therapy as used herein includes a cancer which is not responsive and/or reduced ability of producing a significant response (e.g., partial response and/or complete response) to the therapy. Resistance may be acquired resistance which arises in the course of a treatment method. In some embodiments, the acquired drug resistance is transient and/or reversible drug tolerance. Transient and/or reversible drug resistance to a therapy includes wherein the drug resistance is capable of regaining sensitivity to the therapy after a break in the treatment method. In some embodiments, the acquired resistance is permanent resistance. Permanent resistance to a therapy includes a genetic change conferring drug resistance.

Cancer having sensitivity to a therapy as used herein includes cancer which is responsive and/or capable of producing a significant response (e.g., partial response and/or complete response).

Methods of determining of assessing acquisition of resistance and/or maintenance of sensitivity to a therapy are known in the art and described in the Examples. Drug resistance and/or sensitivity may be determined by (a) exposing a reference cancer cell or cell population to a cancer therapy agent (e.g., targeted therapy, chemotherapy, and/or radiotherapy) in the presence and/or absence of an antagonist of KDM5 and/or (b) assaying, for example, for one or more of cancer cell growth, cell viability, level and/or percentage apoptosis, histone 3 lysine 4 (H3K4) methylation status (e.g., monomethylated, dimethylated, and/or trimethylated), and/or response.

Drug resistance and/or sensitivity may be measured over time and/or at various concentrations of cancer therapy agent (e.g., targeted therapy, chemotherapy, and/or radiotherapy) and/or amount of an antagonist of KDM5. Drug resistance and/or sensitivity further may be measured and/or compared to a reference cell line (e.g., PC9 and/or H1299) including parental cells, drug tolerant persister cells, and/or drug tolerant expanded persister cells of the cell line. In some embodiments, cell viability may be assayed by CyQuant Direct cell proliferation assay. Changes in acquisition of resistance and/or maintenance of sensitivity such as drug tolerance may be assessed by assaying the growth of drug tolerant persisters as described in the Examples and Sharma et al. Changes in acquisition of resistance and/or maintenance of sensitivity such as permanent resistance and/or expanded resisters may be assessed by assaying the growth of drug tolerant expanded persisters as described in the Examples and Sharma et al. In some embodiments, resistance may be indicated by a change in IC₅₀, EC₅₀ or decrease in tumor growth in drug tolerant persisters and/or drug tolerant expanded persisters. In some embodiments, the change is greater than about any of 50%, 100%, and/or 200%. In addition, changes in acquisition of resistance and/or maintenance of sensitivity may be assessed in vivo for examples by assessing response, duration of response, and/or time to progression to a therapy, e.g., partial response and complete response. Changes in acquisition of resistance and/or maintenance of sensitivity may be based on changes in response, duration of response, and/or time to progression to a therapy in a population of individuals, e.g., number of partial responses and complete responses.

In some embodiments of any of the methods, the cancer is a solid tumor cancer. In some embodiments, the cancer is lung cancer, breast cancer, colorectal cancer, colon cancer, melanoma, and/or pancreatic cancer. In some embodiments, the cancer is lung cancer (e.g., non-small cell lung cancer (NSCLC)). In some embodiments, the cancer is breast cancer. In some embodiments, the cancer is CD133 positive. In some embodiments, the cancer is CD24 positive. In some embodiments, the cancer has low levels of H3K4 trimethylation. In some embodiments, the cancer has low levels of H3K4 dimethylation. In some embodiments, the cancer is at risk of developing decreasing levels of H3K4 trimethylation. In some embodiments, the cancer is at risk of developing decreasing levels of H3K4 dimethylation.

The cancer in any of the combination therapies methods described herein when starting the method of treatment comprising the antagonist of KDM5 and the cancer therapy agent (e.g., targeted therapy, chemotherapy, and/or radiotherapy) may be sensitive (examples of sensitive include, but are not limited to, responsive and/or capable of producing a significant response (e.g., partial response and/or complete response)) to a method of treatment comprising the cancer therapy agent (e.g., targeted therapy, chemotherapy, and/or radiotherapy) alone. The cancer in any of the combination therapies methods described herein when starting the method of treatment comprising the antagonist of KDM5 and the cancer therapy agent (e.g., targeted therapy, chemotherapy, and/or radiotherapy) may not be resistant (examples of resistance include, but are not limited to, not responsive and/or reduced ability and/or incapable of producing a significant response (e.g., partial response and/or complete response)) to a method of treatment comprising the cancer therapy agent (e.g., targeted therapy, chemotherapy, and/or radiotherapy) alone.

In some embodiments of any of the methods, the individual according to any of the above embodiments may be a human.

In some embodiments of any of the methods, the combination therapy may be concomitantly administered. In some embodiments of any of the methods, the combination therapies may encompass combined administration (where two or more therapeutic agents are included in the same or separate formulations), and separate administration, in which case, administration of the antagonist of KDM5 and the cancer therapy agent (e.g., targeted therapy, chemotherapy, and/or radiotherapy) can occur prior to, simultaneously, sequentially, concurrently, and/or following, administration of the additional therapeutic agent and/or adjuvant. In some embodiments, the antagonist of KDM5 is administered prior to and/or concurrently with the cancer therapy agent (e.g., targeted therapy, chemotherapy, and/or radiotherapy). In some embodiments, the combination therapy further comprises radiation therapy and/or additional therapeutic agents.

In some embodiments of any of the methods, the antagonist of KDM5 and the cancer therapy agent (e.g., targeted therapy, chemotherapy, and/or radiotherapy) can be administered by any suitable means, including oral, parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

In some embodiments of any of the methods, antagonists of KDM5 (e.g., an antibody, binding polypeptide, and/or binding small molecule) and cancer therapy agents (e.g., targeted therapies, chemotherapy, and/or radiotherapy) described herein may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antagonist of KDM5 and the cancer therapy agent (e.g., targeted therapy, chemotherapy, and/or radiotherapy) not be, but is optionally formulated with one or more agents currently used to prevent or treat the disorder in question. The effective amount of such other agents depends on the amount of the antagonist of KDM5 and the cancer therapy agent (e.g., targeted therapy, chemotherapy, and/or radiotherapy) present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of the antagonist of KDM5 and the cancer therapy agent (e.g., targeted therapy, chemotherapy, and/or radiotherapy) described herein (when used alone or in combination with one or more other additional therapeutic agents) will depend on the type of disease to be treated, the severity and course of the disease, whether the antagonist of KDM5 and the cancer therapy agent (e.g., targeted therapy, chemotherapy, and/or radiotherapy) is administered for preventive or therapeutic purposes, previous therapy, the patient's clinical history and response to the antagonist of KDM5 and the cancer therapy agent (e.g., targeted therapy, chemotherapy, and/or radiotherapy) and the discretion of the attending physician. The antagonist of KDM5 and the cancer therapy agent (e.g., targeted therapy, chemotherapy, and/or radiotherapy) is suitably administered to the patient at one time or over a series of treatments. For repeated administrations over several days or longer, depending on the condition, the treatment would generally be sustained until a desired suppression of disease symptoms occurs. Such doses may be administered intermittently, e.g., every week or every three weeks (e.g., such that the patient receives from about two to about twenty, or e.g., about six doses of the antagonist of KDM5 and the cancer therapy agent (e.g., targeted therapy, chemotherapy, and/or radiotherapy)). An initial higher loading dose, followed by one or more lower doses may be administered. An exemplary dosing regimen comprises administering. However, other dosage regimens may be useful. The progress of this therapy is easily monitored by conventional techniques and assays. In some embodiments, the combination therapy comprises (a) an antagonist of KDM5 and (b) EGFR antagonist. In some embodiments, the combination therapy comprises (a) an antagonist of KDM5 and (b) RAF inhibitor. In some embodiments, the combination therapy comprises (a) an antagonist of KDM5 and (b) PI3K inhibitor. In some embodiments, the combination therapy comprises (a) an antagonist of KDM5 and (b) taxane (e.g., paclitaxel). In some embodiments, the combination therapy comprises (a) an antagonist of KDM5 and (b) platinum agent (e.g., carboplatin or cisplatin). In some embodiments, the combination therapy comprises (a) an antagonist of KDM5, (b) taxane (e.g., paclitaxel), and (c) platinum agent (e.g., carboplatin or cisplatin).

It is understood that any of the above formulations or therapeutic methods may be carried out using an immunoconjugate as the KDM5 and/or cancer therapy agent (e.g., targeted therapy, chemotherapy, and/or radiotherapy).

### III. Therapeutic Compositions

Provided herein are combinations comprising an antagonist of KDM5 and cancer therapy agents (e.g., targeted therapies, chemotherapy, and/or radiotherapy) for use in the methods described herein. In certain embodiments, the combination increases the efficacy the cancer therapy agent (e.g., targeted therapy, chemotherapy, and/or radiotherapy) administered alone. In certain embodiments, the combination delays and/or prevents development of cancer resistance to the cancer therapy agent (e.g., targeted therapy, chemotherapy, and/or radiotherapy). In certain embodiments, the combination extends the period of the cancer therapy agent (e.g., targeted therapy, chemotherapy, and/or radiotherapy) sensitivity in an individual with cancer. In some embodiments, the antagonists of KDM5 and/or the cancer therapy agents (e.g., targeted therapies, chemotherapy, and/or radiotherapy) (e.g., the EGFR antagonist, PI3K antagonists, and/or RAF inhibitors) are an antibody, binding polypeptide, binding small molecule, and/or polynucleotide.

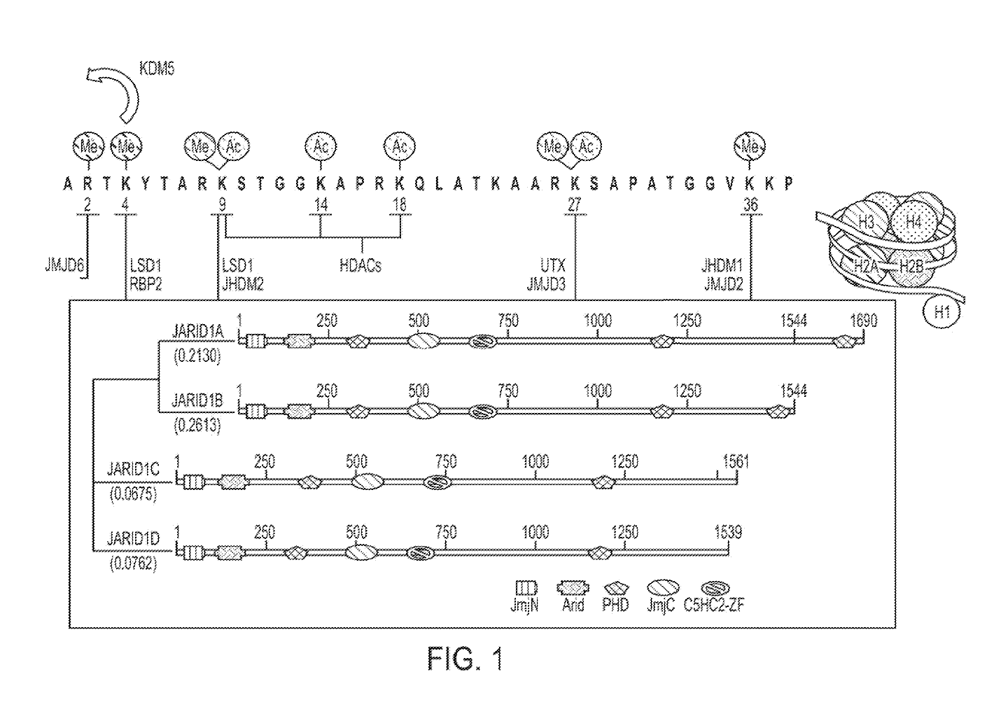

The KDM5/JARID1 family of demethylases in humans contains four members, KDM5A, KDM5B, KDM5C, and KDM5D. As shown in the schematic in FIG. 1, KDM5 family members contain five conserved domains: JmjN, ARID, JmjC, PHD and a C₅HC₂ zinc finger. Amino acid sequences of KDM5A, KDM5B, KDM5C, and KDM5D are known in the art and publicly available, e.g., see UniProtKB/Swiss-Prot (see e.g., KDM5A (e.g., P29375-1 and/or P29375-2), KDM5B (e.g., Q9UGL1-1 and/or Q9UGL1-2), KDM5C (e.g., P41229-1, P41229-2, P41229-3, and/or P41229-4), and/or KDM5D (e.g., Q9BY66-1, Q9BY66-2, and/or Q9BY66-3). In some embodiments of any of the methods, the antagonist of KDM5 is an antagonist of one or more of KDM5A, KDM5B, KDM5C, and KDM5D. In some embodiments, the antagonist of KDM5 is a pan-KDM5 inhibitor (e.g., inhibits KDM5A, KDM5B, KDM5C, and KDM5D). In some embodiments, the antagonist of KDM5 is a KDM inhibitor (e.g., inhibits KDM5 (e.g., one or more of KDM5A, KDM5B, KDM5C, and/or KDM5D) and another KDM (e.g., one or more of KDM1, KDM2, KDM3, KDM4, KDM6, KDM7, and/or KDM8). In some embodiments, the antagonist of KDM5 is an antagonist of KDM5A and/or KDM5B. In some embodiments, the antagonist of KDM5 is a dual antagonist KDM5A and KDM5B. In some embodiments of any of the antagonist of KDM5, the antagonist of KDM5 is a specific KDM5 antagonist, for example, an antagonist specific for KDM5A, an antagonist specific for KDM5B, and/or a dual antagonist specific for KDM5A and KDM5B.

In some embodiments of any of the antagonists of KDM5, the antagonist of KDM5 has a KDM5A IC50 of better than (e.g., less than) about any of 4 μM, 2 μM, 1 μM, 500 nM, 250 nM, 200 nM, 150 nM, 100 nM, 75 nM, 50 nM, and/or 30 nM. Method of determining KDM5A IC50 for a compound are known in the art, which is hereby incorporated by reference in its entirety) and described herein.

In some embodiments of any of the antagonists of KDM5, the antagonist of KDM5 has a KDM2 and/or KDM3 IC50 of greater than about any of 5 μM, 7.5 μM, 10 μM, 15 μM, and/or 20 μM. Method of determining KDM2 and/or KDM3 IC50 for a compound are known in the art and described herein. In some embodiments, the KDM2 is KDM2B. In some embodiments, the KDM3 is KDM3B.

In some embodiments of any of the antagonists of KDM5, the antagonist of KDM5 has a H3K4me³ EC50 of better than (e.g., less than) about any of 25 μM, 15 μM, 10 μM, 7.5 μM, 5 μM, 4 μM, 3.5 μM, 3 μM, 2.5 μM, 2 μM, and/or 1 μM. Method of determining H3K4me³ EC50 for a compound are known in the art (see Sayegh et al. JBC Manuscript M112.419861 (2013), available at world-wide-web jbc.org/cgi/doi/10.1074/jbc.M112.419861 and Kristensen et al. FEBS J. 279:1905-1914 (2012), which are hereby incorporated by reference in their entirety) and described herein.

In some embodiments of any of the antagonists of KDM5, the antagonist of KDM5 inhibits binding of KDM5 (e.g., KDM5A, KDM5B, KDM5C, and/or KDM5D) to α-ketoglutarate. In some embodiments of any of the antagonists of KDM5, the antagonist of KDM5 competes with α-ketoglutarate for binding to KDM5 (e.g., KDM5A, KDM5B, KDM5C, and/or KDM5D). In some embodiments of any of the antagonists, KDM5 inhibits binding of KDM5 (e.g., KDM5A, KDM5B, KDM5C, and/or KDM5D) to α-ketoglutarate by about any of and/or greater than about any of 20%, 30%, 40%, 50%, 60%, 70%, 80% or 90%.

Methods of determining inhibition of binding and/or competition of an antagonist of KDM5 and α-ketoglutarate are known in the art and described, for example, in Kruidenier et al. Nature 488:404-408 (16 Aug. 2012), Kristensen et al. FEBS J. 279:1905-1914 (2012), and Sayegh et al. JBC Manuscript M112.419861 (2013), available at world-wide-web jbc.org/cgi/doi/10.1074/jbc.M112.419861, which are hereby incorporated by reference in their entirety. For example, ketoglutaric acid-α-[1-¹⁴C]-sodium salt, alpha-ketoglutaric acid sodium salt, and HPLC purified peptide may be obtained from commercial sources, e.g., Perkin-Elmer (Wellesley Mass.) and Sigma-Aldrich. Peptides for use in the assay may be fragments of KDM5. For example, a KDM5 peptide for use in the assay can be expressed in, e.g., insect cells, and purified. Enzyme activity is determined by capturing ¹⁴CO₂ using an assay described by Kivirikko and Myllyla (1982, Methods Enzymol. 82:245-304). Assay reactions may contain 50 mM HEPES (pH 7.4), 100 μM α-ketoglutaric acid sodium salt, 0.30 ketoglutaric acid-α-[1-¹⁴C]-sodium salt, 40 μM FeSO₄, 1 mM ascorbate, 1541.8 units/mL Catalase, with or without 50 μM peptide substrate and various concentrations of antagonist of KDM5. Reactions are initiated by addition of KDM5 enzyme.

The peptide-dependent percent turnover is calculated by subtracting percent turnover in the absence of peptide from percent turnover in the presence of substrate peptide. Percent inhibition and IC50 are calculated using peptide-dependent percent turnover at given inhibitor concentrations. Calculation of IC₅₀ values for each inhibitor is conducted using GraFit software (Erithacus Software Ltd., Surrey UK).

In some embodiments of any of the antagonists of KDM5, the antagonist of KDM5 inhibits binding of KDM5 (e.g., KDM5A, KDM5B, KDM5C, and/or KDM5D) to H3. In some embodiments of any of the antagonists of KDM5, the antagonist of KDM5 competes with H3 for binding to KDM5 (e.g., KDM5A, KDM5B, KDM5C, and/or KDM5D). In some embodiments of any of the antagonists, KDM5 inhibits binding of KDM5 (e.g., KDM5A, KDM5B, KDM5C, and/or KDM5D) to H3 by about any of and/or greater than about any of 20%, 30%, 40%, 50%, 60%, 70%, 80% or 90%. In some embodiments, H3 comprises a polypeptide fragment of H3 which comprises H3K4. In some embodiments, H3 comprises H3K4me3. In some embodiments, H3 comprises H3K4me2. In some embodiments, H3 comprises a 21 amino acid polypeptide of H3 comprising H3K4 (for example, H2N-ART(KMe3)QTARKSTGGKAPRKQLA). In some embodiments, the H3 comprises H3K4me3 [ART-K(Me3)-GTARKSTGGKAPRKQLA-GGK(Biotin)], H3K4me2 [ART-K(Me2)-GTARKSTGGKAPRKQLA-GGK(Biotin)], H3K4me1 [ART-K(Me1)-QTARKSTGGKAPRKQLA-GGK(Biotin)].

Methods of determining inhibition of binding and/or competition of an antagonist of KDM5 and a histone such as H3 are known in the art and described, for example, in Kristensen et al. FEBS J. 279:1905-1914 (2012), Kruidenier et al. Nature 488:404-408 (16 Aug. 2012), and Sayegh et al. JBC Manuscript M112.419861 (2013), available at world-wide-web jbc.org/cgi/doi/10.1074/jbc.M112.419861, which are hereby incorporated by reference in their entirety.

In some embodiments of any of the antagonists of KDM5, the antagonist of KDM5 inhibits binding (e.g., interaction and/or association) of KDM5, directly or indirectly, to histone 3 (H3). In some embodiments of any of the antagonists of KDM5, the antagonist of KDM5 inhibits binding (e.g., interaction and/or association) of KDM5, directly or indirectly, to H3 lysine 4 (H3K4). In some embodiments of any of the antagonists of KDM5, the antagonist of KDM5 inhibits binding (e.g., interaction and/or association) of KDM5, directly or indirectly, to H3K4 trimethylated and/or dimethylated (H3K4me³ and/or H3K4me²).

In some embodiments of any of the antagonists of KDM5, the antagonist of KDM5 binds (e.g., interacts and/or associates) with the demethylase catalytic domain of KDM5 (e.g., KDM5A, KDM5B, KDM5C, and/or KDM5D). In some embodiments, the antagonist of KDM5 binds (e.g., interacts and/or associates) to the JmjC domain and/or JmjN domain of KDM5. In some embodiments, the antagonist of KDM5 is an antagonist of KDM5A and the antagonist of KDM5A binds (e.g., interacts and/or associates) to amino acid residues 437-603 (JmjC) and/or amino acid residue 19-60 (JmjN) of SEQ ID NO:1. In some embodiments, the antagonist of KDM5A binds (e.g., interacts and/or associates) to amino acid residues 437-603 (JmjC) of SEQ ID NO:1. In some embodiments, the antagonist of KDM5B binds (e.g., interacts and/or associates) to amino acid residue 483, 486, and/or 571 of SEQ ID NO:1. In some embodiments, the antagonist of KDM5 is an antagonist of KDM5B and the antagonist of KDM5B binds (e.g., interacts and/or associates) to amino acid residues 453-619 (JmjC) and/or amino acid residue 32-73 (JmjN) of SEQ ID NO:2. In some embodiments, the antagonist of KDM5B binds (e.g., interacts and/or associates) to amino acid residues 453-619 (JmjC) of SEQ ID NO:2. In some embodiments, the antagonist of KDM5B binds (e.g., interacts and/or associates) to amino acid residue 499, 502, and/or 587 of SEQ ID NO:2. In some embodiments, the antagonist of KDM5 is an antagonist of KDM5C and the antagonist of KDM5C binds (e.g., interacts and/or associates) to amino acid residues 468-634 (JmjC) and/or amino acid residue 14-55 (JmjN) of SEQ ID NO:3. In some embodiments, the antagonist of KDM5C binds (e.g., interacts and/or associates) to amino acid residues 468-634 (JmjC) of SEQ ID NO:3. In some embodiments, the antagonist of KDM5C binds (e.g., interacts and/or associates) to amino acid residue 514, 517, and/or 602 of SEQ ID NO:3. In some embodiments, the antagonist of KDM5 is an antagonist of KDM5D and the antagonist of KDM5D binds (e.g., interacts and/or associates) to amino acid residues 458-624 (JmjC) and/or amino acid residue 14-55 (JmjN) of SEQ ID NO:4. In some embodiments, the antagonist of KDM5D binds (e.g., interacts and/or associates) to amino acid residues 458-624 (JmjC) of SEQ ID NO:4. In some embodiments, the antagonist of KDM5C binds (e.g., interacts and/or associates) to amino acid residue 504, 507, and/or 592 of SEQ ID NO:4. In some embodiments of any of the antagonists of KDM5, the antagonist of KDM5 inhibits demethylase-catalytic activity.

Examples of antagonist of KDM5 are known in art including, but not limited to those described in, Sayegh et al. JBC Manuscript M112.419861 (2013), available at world-wide-web jbc.org/cgi/doi/10.1074/jbc.M112.419861, Lohse et al., Bioorg. Med. Chem. 19(12):3625-36 (2012), and Kristensen et al. FEBS J. 279:1905-1914 (2012), which are hereby incorporated by reference in their entirety). In some embodiments, the antagonist of KDM5 is JmjC histone demethylase inhibitor including but not limited to 2,4-pyridinedicarboxylic acid (2,4-PDCA), 2,4-pyridine-dicarboxylic acid, catechols, N-phenyl-benzisothiazolinone, and/or 2-(4-methylphenyl)-1,2-benzisothiazol-3(2H)-one (PBIT). In some embodiments, the antagonist of KDM5 is a molecule of a formula below or a pharmaceutically acceptable salt thereof

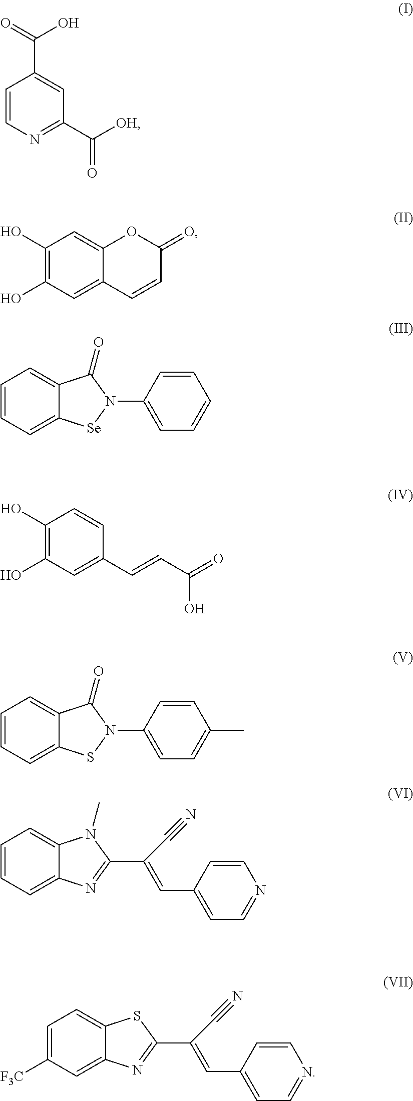

Provided here are also EGFR antagonists useful in the methods described herein. EGFR is meant the receptor tyrosine kinase polypeptide Epidermal Growth Factor Receptor which is described in Ullrich et al, Nature (1984) 309:418425, alternatively referred to as Her-1 and the c-erbB gene product, as well as variants thereof such as EGFRvIII. Variants of EGFR also include deletional, substitutional and insertional variants, for example those described in Lynch et al. (NEJM 2004, 350:2129), Paez et al. (Science 2004, 304:1497), Pao et al. (PNAS 2004, 101:13306). In some embodiment, the EGFR is wild-type EGFR, which generally refers to a polypeptide comprising the amino acid sequence of a naturally occurring EGFR protein. In some embodiments, the EGFR antagonists are an antibody, binding polypeptide, binding small molecule, and/or polynucleotide.

Exemplary EGFR antagonists (anti-EGFR antibodies) include antibodies such as humanized monoclonal antibody known as nimotuzumab (YM Biosciences), fully human ABX-EGF (panitumumab, Abgenix Inc.) as well as fully human antibodies known as E1.1, E2.4, E2.5, E6.2, E6.4, E2.11, E6. 3 and E7.6. 3 and described in U.S. Pat. No. 6,235,883; MDX-447 (Medarex Inc). Pertuzumab (2C4) is a humanized antibody that binds directly to HER2 but interferes with HER2-EGFR dimerization thereby inhibiting EGFR signaling. Other examples of antibodies which bind to EGFR include GA201 (RG7160; Roche Glycart AG), MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF (see WO98/50433, Abgenix); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding; and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). In some embodiments, the anti-EGFR antibody is cetuximab. In some embodiments, the anti-EGFR antibody is panitumumab. In some embodiments, the anti-EGFR antibody is zalutumumab, nimotuzumab, and/or matuzumab.

Anti-EGFR antibodies that are useful in the methods include any antibody that binds with sufficient affinity and specificity to EGFR and can reduce or inhibit EGFR activity. The antibody selected will normally have a sufficiently strong binding affinity for EGFR, for example, the antibody may bind human c-met with a Kd value of between 100 nM-1 pM. Antibody affinities may be determined by a surface plasmon resonance based assay (such as the BIAcore assay as described in PCT Application Publication No. WO2005/012359); enzyme-linked immunoabsorbent assay (ELISA); and competition assays (e.g., RIA's), for example. Preferably, the anti-EGFR antibody of the invention can be used as a therapeutic agent in targeting and interfering with diseases or conditions wherein EGFR/EGFR ligand activity is involved. Also, the antibody may be subjected to other biological activity assays, e.g., in order to evaluate its effectiveness as a therapeutic. Such assays are known in the art and depend on the target antigen and intended use for the antibody. In some embodiments, a EGFR arm may be combined with an arm which binds to a triggering molecule on a leukocyte such as a T-cell receptor molecule (e.g. CD2 or CD3), or Fc receptors for IgG (FcγR), such as FcγRI (CD64), FcγRII (CD32) and FcγRIII (CD16) so as to focus cellular defense mechanisms to the EGFR-expressing cell. Bispecific antibodies may also be used to localize cytotoxic agents to cells which express EGFR. These antibodies possess an EGFR-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g., F(ab′)₂ bispecific antibodies).

Exemplary EGFR antagonists also include binding small molecules such as compounds described in U.S. Pat. No. 5,616,582, U.S. Pat. No. 5,457,105, U.S. Pat. No. 5,475,001, U.S. Pat. No. 5,654,307, U.S. Pat. No. 5,679,683, U.S. Pat. No. 6,084,095, U.S. Pat. No. 6,265,410, U.S. Pat. No. 6,455,534, U.S. Pat. No. 6,521,620, U.S. Pat. No. 6,596,726, U.S. Pat. No. 6,713,484, U.S. Pat. No. 5,770,599, U.S. Pat. No. 6,140,332, U.S. Pat. No. 5,866,572, U.S. Pat. No. 6,399,602, U.S. Pat. No. 6,344,459, U.S. Pat. No. 6,602,863, U.S. Pat. No. 6,391,874, WO9814451, WO9850038, WO9909016, WO9924037, WO9935146, WO0132651, U.S. Pat. No. 6,344,455, U.S. Pat. No. 5,760,041, U.S. Pat. No. 6,002,008, and/or U.S. Pat. No. 5,747,498. Particular binding small molecule EGFR antagonists include OSI-774 (CP-358774, erlotinib, OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); Iressa® (ZD1839, gefitinib, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide); lapatinib (Tykerb, GlaxoSmithKline); ZD6474 (Zactima, AstraZeneca); CUDC-101 (Curis); canertinib (CI-1033); AEE788 (6-[4-[(4-ethyl-1-piperazinyl)methyl]phenyl]-N-[(1R)-1-phenylethyl]-7H-pyrrolo[2,3-d]pyrimidin-4-amine, WO2003013541, Novartis) and PKI166 4-[4-[[(1R)-1-phenylethyl]amino]-7H-pyrrolo[2,3-d]pyrimidin-6-yl]-phenol, WO9702266 Novartis). In some embodiments, the EGFR antagonist is N-(3-ethynylphenyl)-6,7-bis(2-methoxyethoxy)-4-quinazolinamine and/or a pharmaceutical acceptable salt thereof (e.g., N-(3-ethynylphenyl)-6,7-bis(2-methoxyethoxy)-4-quinazolinamine-HCl). In some embodiments, the EGFR antagonist is gefitinib and/or a pharmaceutical acceptable salt thereof. In some embodiments, the EGFR antagonist is lapatinib and/or a pharmaceutical acceptable salt thereof. In some embodiments, the EGFR antagonist is gefitinib and/or erlotinib.

In some embodiments, the EGFR antagonist may be a specific inhibitor for EGFR. In some embodiments, the inhibitor may be a dual inhibitor or pan inhibitor wherein the EGFR antagonist inhibits EGFR and one or more other target polypeptides.

The phosphoinositide 3-kinases (PI3K) are a family of lipid kinases whose primary biochemical function is to phosphorylate the 3-hydroxyl group of phosphoinositides. Examples of PI3K inhibitors are known in the art and include, but are not limited to Wortmannin, LY294002, SF1126 (a small-molecule prodrug, a conjugate of LY294002 linked to an integrin-binding component), NVP-BEZ235 (imidazoquionline derivative), NVP-BGT226, XL765, GDC-0980, PF-04691502, PF-05212384, PKI-587, NVP-BKM120, XL147, PX-866, GDC-0941, GSK615, and/or CAL-101. In some embodiments, the PI3K inhibitor is a compound described in WO2009/114874, WO2009/088990, U.S. Pat. No. 7,511,041, U.S. Pat. No. 7,666,901, U.S. Pat. No. 7,662,977, WO2010/046639, US20100105711, WO2010/037765, US20100087440, WO2010034414, US20100075965, US20100075951, US20100075947, WO2010/038165, WO2010/036380, WO2010/059788, WO2010/049481, WO2009/134825, WO2009/123971, WO2009/099163, and/or WO2009/042607, which are hereby incorporated by reference in their entirety.

Provided here are also RAF inhibitors useful as cancer therapy agents (e.g., targeted therapies, chemotherapy, and/or radiotherapy) in the methods described herein. In some embodiments, the RAF inhibitor is a BRAF inhibitor. In some embodiments, the RAF inhibitor is a CRAF inhibitor. Exemplary BRAF inhibitors are known in the art and include, for example, sorafenib, PLX4720, PLX-3603, dabrafenib (GSK2118436), GDC-0879, RAF265 (Novartis), XL281, AZ628, ARQ736, BAY73-4506, vemurafenib and those described in WO2007/002325, WO2007/002433, WO2009111278, WO2009111279, WO2009111277, WO2009111280 and U.S. Pat. No. 7,491,829. In some embodiments, the BRAF inhibitor is a selective BRAF inhibitor. In some embodiments, the BRAF inhibitor is a selective inhibitor of BRAF V600. In some embodiments, BRAF V600 is BRAF V600E, BRAF V600K, and/or V600D. In some embodiments, BRAF V600 is BRAF V600R. In some embodiments, the BRAF inhibitor is vemurafenib. In some embodiments, the BRAF inhibitor is vemurafenib.

Vemurafenib (RG7204, PLX-4032, CAS Reg. No. 1029872-55-5) has been shown to cause programmed cell death in various cancer call lines, for example melanoma cell lines. Vemurafenib interrupts the BRAF/MEK step on the BRAF/MEK/ERK pathway—if the BRAF has the common V600E mutation. Vemurafenib works in patients, for example in melanoma patients as approved by the FDA, whose cancer has a V600E BRAF mutation (that is, at amino acid position number 600 on the BRAF protein, the normal valine is replaced by glutamic acid). About 60% of melanomas have the V600E BRAF mutation. The V600E mutation is present in a variety of other cancers, including lymphoma, colon cancer, melanoma, thyroid cancer and lung cancer. Vemurafenib has the following structure:

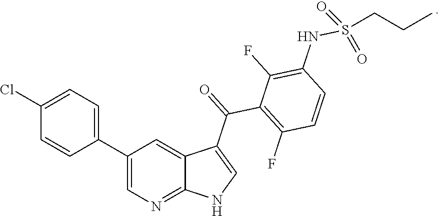 [SMILES: CCCS(=O)(=O)Nc1ccc(F)c(C(=O)c2c[nH]c3ncc(-c4ccc(Cl)cc4)cc23)c1F]

ZELBORAF® (vemurafenib) (Genentech, Inc.) is a drug product approved in the U.S. and indicated for treatment of patients with unresectable or metastatic melanoma with BRAF V600E mutation as detected by an FDA-approved test. ZELBORAF® (vemurafenib) is not recommended for use in melanoma patients who lack the BRAF V600E mutation (wild-type BRAF melanoma).

Provided here are also platinum-based agents useful as cancer therapy agents (e.g., targeted therapies, chemotherapy, and/or radiotherapy) in the methods described herein. Examples of platinum-based agents include, but are not limited to, cisplatin, carboplatin, oxaliplatin, satraplatin, picoplatin, nedaplatin, and/or triplatin. In some embodiments, the platinum-based agent is cisplatin. In some embodiments, the platinum-based agent is carboplatin.

Provided here are also taxanes useful as cancer therapy agents (e.g., targeted therapies, chemotherapy, and/or radiotherapy) in the methods described herein. Taxanes are diterpenes which may bind to tubulin, promoting microtubule assembly and stabilization and/or prevent microtubule depolymerization. Taxanes included herein taxoid 10-deacetylbaccatin III and/or derivatives thereof. Examples to taxanes include, but are not limited to, paclitaxel (i.e., taxol, CAS #33069-62-4), docetaxel (i.e., taxotere, CAS #114977-28-5), larotaxel, cabazitaxel, milataxel, tesetaxel, and/or orataxel. In some embodiments, the taxane is paclitaxel. In some embodiments, the taxane is docetaxel. In some embodiments, the taxane is formulated in Cremophor (e.g., Taxol®) to Tween such as polysorbate 80 (e.g., Taxotere®). In some embodiments, the taxane is liposome encapsulated taxane. In some embodiments, the taxane is a prodrug form and/or conjugated form of taxane (e.g., DHA covalently conjugated to paclitaxel, paclitaxel poliglumex, and/or linoleyl carbonate-paclitaxel). In some embodiments, the paclitaxel is formulated with substantially no surfactant (e.g., in the absence of Cremophor and/or Tween-such as Tocosol Paclitaxel). In some embodiments, the taxane is an albumin-coated nanoparticle (e.g., Abraxane and/or ABI-008). In some embodiments, the taxane is Taxol®.

Provided herein are vinca alkyloids useful as cancer therapy agents (e.g., targeted therapies, chemotherapy, and/or radiotherapy) in the methods described herein. Vinca alkaloids are a set of anti-mitotic and anti-microtubule agents that were originally derived from the Periwinkle plant Catharanthus roseus. Examples of vinca alkyloids include, but are not limited to vinblastine, vincristine, vindesine, and vinorelbine. In some embodiments, the vinca alkyloid is vinorelbine.

Provided herein are nucleoside analogs useful as cancer therapy agents (e.g., targeted therapies, chemotherapy, and/or radiotherapy) in the methods described herein. Examples of nucleoside analogs include, but are not limited to, gemcitabine, fludarabine, 6-mercaptopurine, thiamiprine, thioguanine, ancitabine, azacitidine, 6-azauridine, carmofur, cytarabine, dideoxyuridine, doxifluridine, enocitabine, and/or floxuridine; In some embodiments, the nucleoside analog is gemcitabine.

A. Antibodies

Provided herein isolated antibodies that bind to a polypeptide of interest, such as KDM5 and/or EGFR for use in the methods described herein. In any of the above embodiments, an antibody is humanized. Further, the antibody according to any of the above embodiments is a monoclonal antibody, including a chimeric, humanized or human antibody. In one embodiment, the antibody is an antibody fragment, e.g., a Fv, Fab, Fab′, scFv, diabody, or F(ab′)₂ fragment. In another embodiment, the antibody is a full length antibody, e.g., an “intact IgG1” antibody or other antibody class or isotype as defined herein.

In a further aspect, an antibody according to any of the above embodiments may incorporate any of the features, singly or in combination, as described in Sections below:

1. Antibody Affinity

In certain embodiments, an antibody provided herein has a dissociation constant (Kd) of ≦1 μM, ≦100 nM, ≦10 nM, ≦1 nM, ≦0.1 nM, ≦0.01 nM, or ≦0.001 nM (e.g., 10⁻⁸M or less, e.g., from 10⁻⁸ M to 10⁻¹³ M, e.g., from 10⁻⁹ M to 10⁻¹³ M). In one embodiment, Kd is measured by a radiolabeled antigen binding assay (RIA). In one embodiment, the RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity-determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMab® technology; U.S. Pat. No. 7,041,870 describing K-M MOUSE® technology, and U.S. Patent Application Publication No. US 2007/0061900, describing VelociMouse® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Hist. & Histopath., 20(3):927-937 (2005) and Vollmers and Brandlein, Methods Find Exp. Clin. Pharmacol., 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. Methods Mol. Biol. 178:1-37 (O'Brien et al., ed., Human Press, Totowa, N. J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, Methods Mol. Biol. 248:161-175 (Lo, ed., Human Press, Totowa, N. J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein is a multispecific antibody, e.g., a bispecific antibody. Multispecific antibodies are monoclonal antibodies that have binding specificities for at least two different sites. In certain embodiments, one of the binding specificities is a polypeptide of interest, such as KDM5 and/or EGFR and the other is for any other antigen. In certain embodiments, bispecific antibodies may bind to two different epitopes of a polypeptide of interest, such as KDM5 and/or EGFR. Bispecific antibodies may also be used to localize cytotoxic agents to cells which express a polypeptide of interest, such as KDM5 and/or EGFR. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to a polypeptide of interest, such as KDM5 and/or EGFR as well as another, different antigen (see, US 2008/0069820, for example).

7. Antibody Variants

a) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

b) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII) only, whereas monocytes express Fc(RI), Fc(RII) and Fc(RIII). FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).) In certain embodiments, an antibody variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. No. 5,648,260; U.S. Pat. No. 5,624,821; and WO 94/29351 concerning other examples of Fc region variants.

c) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

B. Immunoconjugates

Further provided herein are immunoconjugates comprising antibodies which bind a polypeptide of interest such as KDM5 or EGFR, conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes for use in the methods described herein.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example Tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

C. Binding Polypeptides

Binding polypeptides are polypeptides that bind a polypeptide of interest, including to KDM5 and/or EGFR are also provided for use in the methods described herein. In some embodiments, the binding polypeptides are KDM5 antagonists and/or EGFR antagonists. Binding polypeptides may be chemically synthesized using known polypeptide synthesis methodology or may be prepared and purified using recombinant technology. Binding polypeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such binding polypeptides that are capable of binding, preferably specifically, to a target, e.g., KDM5 or EGFR, as described herein. In some embodiments, the binding polypeptide inhibits KDM5 demethylase activity. In some embodiments, the KDM5 is one or more of KDM5A, KDM5B, KDM5C, and/or KDM5D. In some embodiments, the KDM5 is KDM5A and/or KDM5B.

Binding polypeptides may be identified without undue experimentation using well known techniques. In this regard, it is noted that techniques for screening polypeptide libraries for binding polypeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

D. Binding Small Molecules

Provided herein are binding small molecules for use as a binding small molecule antagonist of a polypeptide of interest such as KDM5 and/or EGFR for use in the methods described above. In some embodiments, the binding small molecule antagonist inhibits KDM5 demethylase activity. In some embodiments, the KDM5 is one or more of KDM5A, KDM5B, KDM5C, and/or KDM5D. In some embodiments, the KDM5 is KDM5A and/or KDM5B.

Binding small molecules are preferably organic molecules other than binding polypeptides or antibodies as defined herein that bind, preferably specifically, to KDM5 and/or EGFR as described herein.

Binding small molecules may be identified and chemically synthesized using known methodology (see, e.g., PCT Publication Nos. WO00/00823 and WO00/39585). Binding small molecules may also be identified as those binding to the JmjC domain and/or JmjN domain of the KDM5. Binding small molecules are usually less than about 2000 daltons in size, alternatively less than about 1500, 750, 500, 250 or 200 daltons in size, wherein such small molecules that are capable of binding, preferably specifically, to a polypeptide as described herein may be identified without undue experimentation using well known techniques. In this regard, it is noted that techniques for screening organic small molecule libraries for molecules that are capable of binding to a polypeptide of interest are well known in the art (see, e.g., PCT Publication Nos. WO00/00823 and WO00/39585). Binding organic small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

E. Antagonist Polynucleotides

Provided herein are also polynucleotide antagonists for use in the methods described herein. The polynucleotide may be an antisense nucleic acid and/or a ribozyme. The antisense nucleic acids comprise a sequence complementary to at least a portion of an RNA transcript of a gene of interest, such as KDM5 gene described herein and/or EGFR gene. In some embodiments, the KDM5 is one or more of KDM5A, KDM5B, KDM5C, and/or KDM5D. In some embodiments, the KDM5 is KDM5A and/or KDM5B. However, absolute complementarity, although preferred, is not required.

A sequence “complementary to at least a portion of an RNA,” referred to herein, means a sequence having sufficient complementarity to be able to hybridize with the RNA, forming a stable duplex; in the case of double stranded antisense nucleic acids, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with a RNA it may contain and still form a stable duplex (or triplex as the case may be). One skilled in the art can ascertain a tolerable degree of mismatch by use of standard procedures to determine the melting point of the hybridized complex.

Polynucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′-non-translated, non-coding regions of the gene, could be used in an antisense approach to inhibit translation of endogenous mRNA. Polynucleotides complementary to the 5′ untranslated region of the mRNA should include the complement of the AUG start codon. Antisense polynucleotides complementary to mRNA coding regions are less efficient inhibitors of translation but could be used in accordance with the invention. Whether designed to hybridize to the 5′-, 3′- or coding region of an mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Examples of short hairpin RNA (shRNA) that may be effective include shRNA based on, for KDM5A (the mature antisense) TGCCGTTTCCATTATTCAA (SEQ ID NO:5), TCAGTCATGAGAGTCAATT (SEQ ID NO:6), and TACTAGAGGACTTCACACT (SEQ ID NO:7) and for KDM5B (the mature antisense) TCGAAGCTTCAATGCATTC (SEQ ID NO:8), TATCGAAGTGCATCTCCCT (SEQ ID NO:9), and TTCGGAATAGGATGTGTCT (SEQ ID NO:10).

F. Antibody and Binding Polypeptide Variants

In certain embodiments, amino acid sequence variants of the antibodies and/or the binding polypeptides provided herein are contemplated. For example, it may be desirable to improve the binding affinity and/or other biological properties of the antibody and/or binding polypeptide. Amino acid sequence variants of an antibody and/or binding polypeptides may be prepared by introducing appropriate modifications into the nucleotide sequence encoding the antibody and/or binding polypeptide, or by peptide synthesis. Such modifications include, for example, deletions from, and/or insertions into and/or substitutions of residues within the amino acid sequences of the antibody and/or binding polypeptide. Any combination of deletion, insertion, and substitution can be made to arrive at the final construct, provided that the final construct possesses the desired characteristics, e.g., antigen-binding.

In certain embodiments, antibody variants and/or binding polypeptide variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody and/or binding polypeptide of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

G. Antibody and Binding Polypeptide Derivatives

In certain embodiments, an antibody and/or binding polypeptide provided herein may be further modified to contain additional nonproteinaceous moieties that are known in the art and readily available. The moieties suitable for derivatization of the antibody and/or binding polypeptide include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody and/or binding polypeptide may vary, and if more than one polymer are attached, they can be the same or different molecules. In general, the number and/or type of polymers used for derivatization can be determined based on considerations including, but not limited to, the particular properties or functions of the antibody and/or binding polypeptide to be improved, whether the antibody derivative and/or binding polypeptide derivative will be used in a therapy under defined conditions, etc.

In another embodiment, conjugates of an antibody and/or binding polypeptide to nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody and/or binding polypeptide-nonproteinaceous moiety are killed.

### IV. Methods of Screening and/or Identifying Antagonists of KDM5 with Desired Function

Additional antagonists of a polypeptide of interest, such as KDM5 and/or EGFR for use in the methods described herein, including antibodies, binding polypeptides, and/or small molecules have been described above. Additional antagonists of such as anti-KDM5 antibodies, binding polypeptides, and/or binding small molecules provided herein may be identified, screened for, or characterized for their physical/chemical properties and/or biological activities by various assays known in the art.

In certain embodiments, a computer system comprising a memory comprising atomic coordinates of KDM5 polypeptide are useful as models for rationally identifying compounds that a ligand binding site of KDM5. Such compounds may be designed either de novo, or by modification of a known compound, for example. In other cases, binding compounds may be identified by testing known compounds to determine if the “dock” with a molecular model of KDM5. Such docking methods are generally well known in the art.

The KDM5 crystal structure data can be used in conjunction with computer-modeling techniques to develop models of binding of various KDM5-binding compounds by analysis of the crystal structure data. The site models characterize the three-dimensional topography of site surface, as well as factors including van der Waals contacts, electrostatic interactions, and hydrogen-bonding opportunities. Computer simulation techniques are then used to map interaction positions for functional groups including but not limited to protons, hydroxyl groups, amine groups, divalent cations, aromatic and aliphatic functional groups, amide groups, alcohol groups, etc. that are designed to interact with the model site. These groups may be designed into a pharmacophore or candidate compound with the expectation that the candidate compound will specifically bind to the site. Pharmacophore design thus involves a consideration of the ability of the candidate compounds falling within the pharmacophore to interact with a site through any or all of the available types of chemical interactions, including hydrogen bonding, van der Waals, electrostatic, and covalent interactions, although in general, pharmacophores interact with a site through non-covalent mechanisms.

The ability of a pharmacophore or candidate compound to bind to KDM5 polypeptide can be analyzed in addition to actual synthesis using computer modeling techniques. Only those candidates that are indicated by computer modeling to bind the target (e.g., KDM5 polypeptide binding site) with sufficient binding energy (in one example, binding energy corresponding to a dissociation constant with the target on the order of 10⁻² M or tighter) may be synthesized and tested for their ability to bind to KDM5 polypeptide and to inhibit KDM5, if applicable, enzymatic function using enzyme assays known to those of skill in the art and/or as described herein. The computational evaluation step thus avoids the unnecessary synthesis of compounds that are unlikely to bind KDM5 polypeptide with adequate affinity.

KDM5 pharmacophore or candidate compound may be computationally evaluated and designed by means of a series of steps in which chemical entities or fragments are screened and selected for their ability to associate with individual binding target sites on KDM5 polypeptide. One skilled in the art may use one of several methods to screen chemical entities or fragments for their ability to associate with KDM5 polypeptide, and more particularly with target sites on KDM5 polypeptide. The process may begin by visual inspection of, for example a target site on a computer screen, based on the KDM5 polypeptide coordinates, or a subset of those coordinates known in the art.

To select for an antagonist which induces cancer cell death, loss of membrane integrity as indicated by, e.g., propidium iodide (PI), trypan blue or 7AAD uptake may be assessed relative to a reference. A PI uptake assay can be performed in the absence of complement and immune effector cells. A tumor cells are incubated with medium alone or medium containing the appropriate combination therapy. The cells are incubated for a 3-day time period. Following each treatment, cells are washed and aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN® flow cytometer and FACSCONVERT® CellQuest software (Becton Dickinson). Those antagonists that induce statistically significant levels of cell death compared to media alone and/or monotherapy as determined by PI uptake may be selected as cell death-inducing antibodies, binding polypeptides or binding small molecules.

In some embodiments of any of the methods of screening and/or identifying, the candidate antagonist of KDM5 is an antibody, binding polypeptide, binding small molecule, or polynucleotide. In some embodiments, the antagonist of KDM5 is an antibody. In some embodiments, the antagonist of KDM5 is a binding small molecule. In some embodiments, the KDM5 antagonist inhibits KDM5 demethylase activity. In some embodiments, the KDM5 is one or more of KDM5A, KDM5B, KDM5C, and/or KDM5D. In some embodiments, the KDM5 is KDM5A and/or KDM5B.

### V. Pharmaceutical Formulations

Pharmaceutical formulations of an antagonist of KDM5 and/or a cancer therapy agent (e.g., targeted therapy, chemotherapy, and/or radiotherapy) as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. In some embodiments, the antagonist of KDM5 and/or targeted therapy is a binding small molecule, an antibody, binding polypeptide, and/or polynucleotide. In some embodiments, the cancer therapy agent is EGFR antagonist. In some embodiments, the cancer therapy agent is a taxane. In some embodiments, the taxane is paclitaxel. In some embodiments, the taxane is docetaxel.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antagonist of KDM5 and/or cancer therapy agent (e.g., targeted therapy, chemotherapy, and/or radiotherapy) which matrices are in the form of shaped articles, e.g., films, or microcapsules.

The formulations to be used for in vivo administration are generally sterile. Sterility may be readily accomplished, e.g., by filtration through sterile filtration membranes.

### VI. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antagonist of KDM5 described herein. The label or package insert indicates that the composition is used for treating the condition of choice. Moreover, the article of manufacture may comprise (a) a first container with a composition contained therein, wherein the composition comprises an antagonist of KDM5 and (b) a second container with a composition contained therein, wherein the composition comprises a cancer therapy agent (e.g., targeted therapy, chemotherapy, and/or radiotherapy).

In some embodiments, the article of manufacture comprises a container, a label on said container, and a composition contained within said container; wherein the composition includes one or more reagents (e.g., primary antibodies that bind to one or more biomarkers or probes and/or primers to one or more of the biomarkers described herein), the label on the container indicating that the composition can be used to evaluate the presence of one or more biomarkers in a sample, and instructions for using the reagents for evaluating the presence of one or more biomarkers in a sample. The article of manufacture can further comprise a set of instructions and materials for preparing the sample and utilizing the reagents. In some embodiments, the article of manufacture may include reagents such as both a primary and secondary antibody, wherein the secondary antibody is conjugated to a label, e.g., an enzymatic label. In some embodiments, the article of manufacture one or more probes and/or primers to one or more of the biomarkers described herein.

In some embodiments of any of the article of manufacture, the antagonist of KDM5 and/or the cancer therapy agent is an antibody, binding polypeptide, binding small molecule, or polynucleotide. In some embodiments, the cancer therapy agent is a taxane. In some embodiments, the taxane is paclitaxel. In some embodiments, the cancer therapy agent is an EGFR antagonist. In some embodiments, the antagonist of KDM5 and/or EGFR antagonist is a binding small molecule. In some embodiments, the EGFR binding small molecule antagonist is erlotinib. In some embodiments, the antagonist of KDM5 and/or EGFR antagonist is an antibody. In some embodiments, the antibody is a monoclonal antibody. In some embodiments, the antibody is a human, humanized, or chimeric antibody. In some embodiments, the antibody is an antibody fragment and the antibody fragment binds KDM5 and/or inhibitor. In some embodiments, the KDM5 antagonist inhibits KDM5 demethylase activity. In some embodiments, the KDM5 is one or more of KDM5A, KDM5B, KDM5C, and/or KDM5D. In some embodiments, the KDM5 is KDM5A and/or KDM5B.

The article of manufacture in this embodiment of the invention may further comprise a package insert indicating that the compositions can be used to treat a particular condition. In some embodiments, the package insert comprises instructions for administering the KDM5 antagonist prior to and/or concurrently with the cancer therapy agent (e.g., targeted therapy, chemotherapy, and/or radiotherapy). Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Other optional components in the article of manufacture include one or more buffers (e.g., block buffer, wash buffer, substrate buffer, etc.), other reagents such as substrate (e.g., chromogen) which is chemically altered by an enzymatic label, epitope retrieval solution, control samples (positive and/or negative controls), control slide(s) etc.

It is understood that any of the above articles of manufacture may include an immunoconjugate described herein in place of or in addition to an antagonist of KDM5 and a cancer therapy agent (e.g., targeted therapy, chemotherapy, and/or radiotherapy).

### EXAMPLES

The following are examples of methods and compositions of the invention. It is understood that various other embodiments may be practiced, given the general description provided above. Results are also presented and described in the Figures and Figure Legends.

### Example 1

### Materials and Methods

Cell Culture

All cells are maintained in RPMI media (high glucose) supplemented with 5% Fetal Bovine Serum (FBS) and L-glutamine under 5% CO₂ at 37° C.

Cell Survival Assays

3×10⁴ cells were plated in each well of a 12-well cluster dish. 24 hours after plating, media was removed and replaced with media containing drugs. Fresh media was replaced every 2 days until untreated cells reached confluence. Media was then removed, cells were washed with Phosphate Buffered Saline (PBS), and then fixed for 15 min with 4% formaldehyde in PBS. Cells were then washed with PBS and stained with the fluorescent nucleic acid stain, Syto60 (1 nM in PBS; Molecular Probes) for 15 min. Dye was removed, cell monolayers were washed with PBS, and fluorescence quantitation was carried out at 700 nm with an Odyssey Infrared Imager (Li-Cor Biosciences).

Generation of Drug-Tolerant Persisters (DTPs)

Drug-sensitive cells were treated with relevant drug as described herein at concentrations exceeding 100 times the established IC₅₀ values, for three rounds, with each treatment lasting 72 hours. Viable cells remaining attached on the dish at the end of the third round of relevant drug treatment were considered to be DTPs, and were collected for analysis.

Specifically for carboplatin and paclitaxel DTPs, Cells were plated and grown to 60-70% confluency then treated with Carboplatin (5.38 uM) and Paclitaxel (1.25 uM) for 5 cycles of 24 hours on 48 hours off drug. DTPs were collected and analyzed 1 week after the final dose of chemotherapy.

Gamma Irradiation

For Gamma irradiation the cells were treated for 5 days with inhibitors replated at 500,000 cells with the inhibitor. After cell attachment (approximately 8 hours), the cells were irradiated, and cells were counted 5 days after irradiation.

siRNA and shRNA Knock-Down

For siRNA knock-down, cells were reverse transfected in black 96 well clear bottom plates (Corning, catalog #3603) at 1000 cell per well using 0.0625 ul of DharmaFECT 1 transfection lipid (Dharmacon, catalog #T-2001) and single siRNA (Dharmacon siGENOME) at 12.5 nM final concentration. Cells were subsequently transfected for 48-72 hours before replacing the transfection media by either 1 uM relevant drug treatment in media or media alone. After 72 hours of incubation the media+/−drug was then replaced with fresh media to enable recovery of the drug tolerant persisters (DTPs) that survived after the relevant drug treatment (recovery phase). After 3 days recovery phase, final cell viability was measured using CyQUANT Direct cell proliferation assay (Molecular Probes) according to the manufacturer protocol. CyQUANT fluorescent signal was detected using a GE IN Cell Analyzer 2000 (4× objective) and quantified as number of cell per well using an image analysis algorithm developed using GE Developer Tollbox 1.9.1. Data were subsequently processed in Microsoft Excel, and each cell line run twice in completely independent conditions.

For KDM5 short hairpin RNA (shRNA) experiments, the KDM5 shRNA were obtained from Dharmacon (Thermo Scientific) with the following sequences: KDM5A shRNA1-TGCCGTTTCCATTATTCAA (SEQ ID NO:5) (mature antisense), KDM5A shRNA2-TCAGTCATGAGAGTCAATT (SEQ ID NO:6) (mature antisense), KDM5A shRNA3-TACTAGAGGACTTCACACT (SEQ ID NO:7) (mature antisense), KDM5B shRNA1-TCGAAGCTTCAATGCATTC (SEQ ID NO:8) (mature antisense), KDM5B shRNA2-TATCGAAGTGCATCTCCCT (SEQ ID NO:9) (mature antisense), and KDM5B shRNA3-TTCGGAATAGGATGTGTCT (SEQ ID NO:10) (mature antisense). For KDM5A siRNA experiments, the KDM5A siRNAs were obtained from Dharmacon (Thermo Scientific) with the following sequences: KDM5A siRNA1-GCAAAUGAGACAACGGAAA (SEQ ID NO:11), KDM5A siRNA2-UGACAAUGGUGGACCGCAU (SEQ ID NO:12), KDM5A siRNA3-CAACACAUAUGGCGGAUUU (SEQ ID NO:13), and KDM5A siRNA4-GGAUGAACAUUCUGCCGAA (SEQ ID NO:14).

Binding Small Molecule Inhibitor Experiments

Generally, for KDM5 inhibitor experiments cells were treated with active or inactive compound for 3-5 days prior to chemotherapy treatment and were maintained on drug for the duration of the study. The structures of CPI-382 and CPI-383 are provided below.

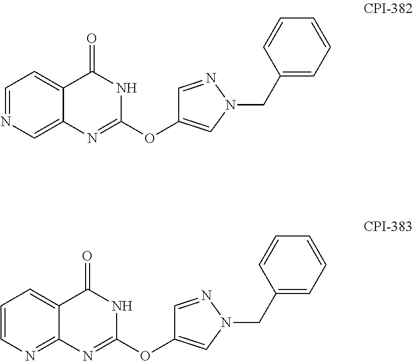 [SMILES: O=c1[nH]c(Oc2cnn(Cc3ccccc3)c2)nc2cnccc12 ; O=c1[nH]c(Oc2cnn(Cc3ccccc3)c2)nc2ncccc12]

Cell Harvesting and Protein Analysis

Cell lysates were prepared in Laemmli sample buffer and analyzed by immunoblotting as described previously. Cell lystates were analyzed using commercial antibodies against modifications on H3 (Abcam, Active Motif, and Cell Signaling Technologies).

Mass Spectrometry Sample Preparation

Samples with 10 million cells were lysed and histones were isolated from cell lysates using the Active Motif Histone Purification Kit (world wide web activemotif.com/catalog/171.html). Protein quantitation post-isolation was performed using the Qubit fluorescence platform (Invitrogen). The target yield was at least 20 μg or greater of purified histone per 5 million cells. The samples were then derivatized and binary comparisons using d0/d10 propionic anhydride and trypsin digestion was conducted. Specifically, 5 μg aliquot of each sample was derivatized with d0 propionic anhydride to block lysine and monomethylated lysine residues. The control sample utilized 15 μg. Samples were digested with trypsin. Control sample were re-derivatized (on exposed peptide N-termini) with d0 propionic anhydride. Test samples were re-derivatized (on exposed N-termini) with d10 propionic anhydride. Each test sample was independently pooled 1:1 with control sample. Then the samples were subjected to multi-enzyme digestion. A suite of three enzymes per sample was employed to generate large peptides around the PTM sites to be characterized, and concomitant overlapping sequence coverage around all sites.

Mass Spectrometry

Peptide digests were analyzed by nano LC/MS/MS in data-dependent mode on a LTQ Orbitrap Velos tandem mass spectrometer. Data was acquired using CID, HCD and ETD fragmentation regimes. Upon data acquisition, database searching using Mascot (Matrix Science) was used to determine acetylation, methylation, dimethlyation, trimethylation, phosphorylation and ubiquitination. Manual data analysis including de novo sequencing was used to confirm putative in-silico assignments and interrogate raw data for modified peptides not matched in Mascot. Accurate mass full scan LC/MS data was integrated to determine relative abundance of modified peptides between samples. Trypsin-digested propionylated samples were quantitated within each LC/MS run by comparing d0/d5 pairs (according to the work of Garcia et al., JPR, 8, 5367-5374 (2009)). Alternate enzyme samples were quantitated label-free between LC/MS runs.

KDM2B Demethylase Assays (MassSpec Assay)

Full length recombinant KDM2B protein was purified from Sf9 insect cells to near homogeneity. The demethylation reaction buffer contained 50 mM TrisCl pH 7.5, 0.02% Triton X-100, 0.001% BSA, 1 mM ascorbate (Cat# A4034, Sigma Aldrich), 1 mM TCEP, and 50 μM Fe₂(NH4)₂(SO4)₂ (Cat# F1543, Sigma Aldrich). In a 25 μL demethylation reaction system, 100 nM recombinant KDM2B and 2 μM biotinylated H3K36me2 peptide (26-46 aa) were incubated with compounds for 10 minutes, and then 2.0 μM α-ketoglutarate (# K2010, Sigma Aldrich) was added to initiate the reaction. (All reagent concentrations are final reagent concentrations.) Reactions were incubated for 40 minutes at room temperature, and then 25 μl of 1% formic acid was added to quench the reactions. After termination, plates were sealed and frozen at −80° C. for analysis.

KDM3B Demethylase Assays (MassSpec Assay)

Full length recombinant Flag tagged KDM3B protein was purified from Sf9 insect cells. The demethylation reaction buffer contained 50 mM TrisCl pH 7.4, 0.01% Triton X-100, 0.05 mg/mL BSA, 0.4 mM ascorbate (Cat# A4034, Sigma Aldrich), 1 mM TCEP (Cat# D9779, Sigma Aldrich), 1.4 μM α-ketoglutarate (# K2010, Sigma Aldrich) and 40 μM Fe₂(NH₄)₂(SO₄)₂ (Cat# F1543, Sigma Aldrich). In a 25 μL demethylation reaction system, 15 nM recombinant KDM3B and was incubated with compounds for 10 minutes in the above buffer, and then 1.4 μM α-ketoglutarate (# K2010, Sigma Aldrich) and 2.5 μM biotinylated H3K9me1 peptide (1-21 aa) were added to initiate the reaction. (All reagent concentrations are final reagent concentrations.) Reactions were incubated for 15 minutes at room temperature, and then quenched by addition of an equal volume of 1% formic acid. After termination, plates were sealed and frozen at −80° C. for analysis.

KDM3B Demethylase Assays (TR-FRET Assay)

Full length recombinant Flag tagged KDM3B protein was purified from Sf9 insect cells. The demethylation reaction buffer contained 50 mM TrisCl pH 7.3, 0.02% Triton X-100, 0.05 mg/mL BSA, 0.4 mM ascorbate (Cat# A4034, Sigma Aldrich), 1 mM TCEP (Cat# D9779, Sigma Aldrich), and 40 μM Fe₂(NH₄)₂(SO₄)₂ (Cat# F1543, Sigma Aldrich). In a 10 μL demethylation reaction system, 0.5 nM recombinant KDM3B and 0.1 μM biotinylated H3K9me1 peptide (1-21 aa) were incubated with compounds for 10 minutes in the above buffer and then 1.4 μM α-ketoglutarate (# K2010, Sigma Aldrich) was added to initiate the reaction. (All reagent concentrations are final reagent concentrations.) Reactions were incubated for 15 minutes at room temperature, and then quenched by addition of an equal volume of detection solution (50 mM TrisCl pH 7.3, 0.02% Triton X-100, 0.05 mg/mL BSA, 0.05 mM EDTA, 0.2 mM NOG, 0.05 uM Ulight-SA (Perkin-Elmer Corp.), and 0.2 nM PE Eu-anti-H3K9me0 antibody (Perkin-Elmer Corp.)). Plates were incubated for 30 minutes and read on the Perkin-Elmer Envision instrument.

KDM5A Demethylase Assays (MassSpec Assay)

Full length recombinant Flag tagged KDM5A protein was purified from Sf9 insect cells. The demethylation reaction buffer contained 50 mM TrisCl pH 7.4, 0.01% Triton X-100, 0.025 mg/mL BSA, 1 mM ascorbate (Cat# A4034, Sigma Aldrich), 2 mM TCEP (Cat# D9779, Sigma Aldrich), 2.0 μM α-ketoglutarate (# K2010, Sigma Aldrich) and 50 μM Fe₂(NH₄)₂(SO₄)₂ (Cat# F1543, Sigma Aldrich). In a 25 μL demethylation reaction system, 20 nM recombinant KDM5A and was incubated with compounds for 10 minutes in the above buffer, and then 2.0 α-ketoglutarate (# K2010, Sigma Aldrich), 4.0 μM biotinylated H3K9me1 peptide (1-21 aa), and Fe₂(NH₄)₂(SO₄)₂ were added to initiate the reaction. (All reagent concentrations are final reagent concentrations.) Reactions were incubated for 30 minutes at room temperature, and then quenched by addition of an equal volume of 1% formic acid. After termination, plates were sealed and frozen at −80° C. for analysis.

High Throughput Mass Spectrometry (HT-MS) Analysis for Demethylase Assays

All the reactions were read by RapidFire™ HT-MS platform developed at Agilent (formerly BioCius Inc), and described in detail (Assay and Drug Development Technologies, 2004; 2(4): 373-381). Briefly, plates were thawed and immediately analyzed using RapidFire™ system coupled to a Sciex API4000 triple quadrapole mass spectrometer. The samples were delivered directly from the plate to a clean-up cartridge (Agilent column A) to remove nonvolatile assay components with 0.1% formic acid in a 3-sec wash cycle. The peptide substrate and demethylated product were coeluted to the mass spectrometer with 80% acetonitrile, 0.1% formic acid. Both the substrate and product signals were read at their +5 charge species, and the conversion from substrate to product assessed.

JARID Cell Assay (Measuring Global H3K4Me3 Changes)

Cells were plated in 10% DMEM in 96-well imaging plates (BD Falcon #353219). After approximately 24 hours, media was changed to 0% DMEM and compounds were added as appropriate in 0% DMEM. Twenty four hours after compound addition, cell were fixed in 4% PFA for 10 minutes at RT, washed once with PBS, and ice-cold methanol was added for 10 minutes at −20° C. Cell were then washed and PBS added. Plates were stored in 4° C. until stained.

Cell were stained for H3K4me3 staining by blocking with blocking solution (1% BSA, 5% normal goat serum, 0.3% Triton X-100 in PBS, filter sterile) for 30 minutes at RT and followed by addition of primary antibody mix (blocking solution with Rabbit anti-H3K4me3 (Cell Signaling #9751) 1:200 and/or Mouse anti-total histone (Millipore #MAB3422) 1:300) for 45 minutes at RT. Cells were washed in PBS followed by the addition of secondary antibody mix (blocking solution with Goat anti-rabbit IgG Alexa Fluor 488 (Invitrogen #A11034) 1:500, Goat anti-mouse IgG Alexa Fluor 594 (Invitrogen #A11032) 1:500, and/or Hoechst 33342 (Invitrogen #H3570) 1:4000) for 45 minutes at RT in the dark. Cells were subsequently washed with PBS and stored in PBS (D100) in 4° C. until imaging. Images of cells were acquired on ImageXpress.

H3K4me3 MSD

Cells were rinsed with PBS and MSD Buffer AT (10 mM HEPES, pH 7.9, 5 mM MgCl2, 0.25M sucrose, Benzonase (1:10000), 1% Triton X-100 supplemented with fresh 1× Protease Inhibitor cocktail and 1 mM PMSF/AEBSF) was added. Cells were lysed for 30 minutes and then 10 uL 5M NaCl was added and allowed to lyse on ice for another 15 minutes. Lysates were rinse with 150 uL ice-cold NO Salt NO detergent buffer (20 mM Tris pH 7.5, 1 mM EDTA, 1 mM EGTA, supplemented with fresh 1× Protease Inhibitor cocktail and 1 mM PMSF).

MSD plates (Catalog #L15XA-3) were then coated with Capture Antibody Anti-histone (Millipore Catalog # MAB3422) for H3K4me3: 2 ug/mL final concentration and/or plates testing for H3: 1 ug/mL final concentration. MSD plates were then blocked with 5% Blocker A (MSD Catalog #R93AA-2). Lysates to MSD plates were subsequently transferred, sealed and incubated with shaking at RT for 2:30 hours and then incubated with detection antibody in 1% Blocker A in TBST for 30 min (anti-Histone H3 (#4499 from Cell Signaling) at 0.125 ug/mL and/or anti-K4Me3 (#9751 from Cell Signaling) at 1 ug/mL). Sulfo-tag rabbit antibody (MSD Catalog# R32AB-1) in 1% Blocker A in TBST was added and incubated for 1 h at RT. Anti-K4Me3 (#9751) 1 ug/mL and Anti-Histone H3 (#4499) were used at 0.5 ug/mL. 1× Read Buffer (MSD Catalog #R92TD-3) was added and read on MSD SECTOR® Imager 2400. Data was analyzed data using MACRO template provided using samples treated with DMSO as 0% or Min. Data was also normalized to total histone H3 by calculating the ratio of methyl mark level in each well to the corresponding Histone H3 level and normalizing and averaging the same.

### Results

KDM5 is a demethylase capable of removing tri- and di-methyl marks from lysine 4 of H3. KDM5 is also known as JARID1, and the KDM5/JARID1 family of demethylases in humans contains four members, KDM5A, KDM5B, KDM5C, and KDM5D. As shown in FIG. 1, KDM5 family members contain five conserved domains: JmjN, ARID, JmjC, PHD and a C5HC2 zinc finger.

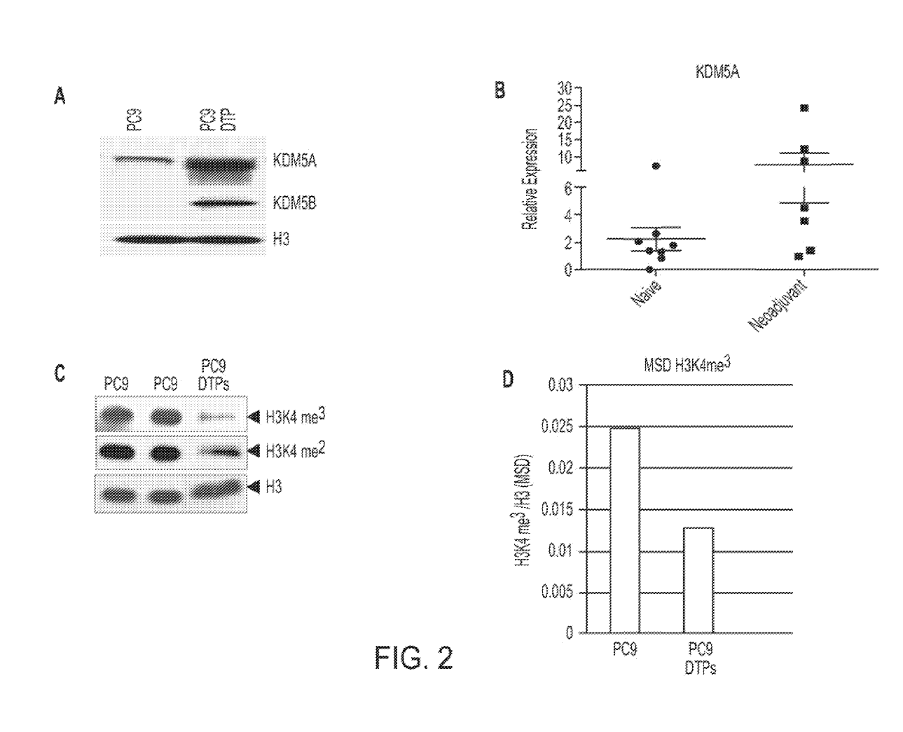

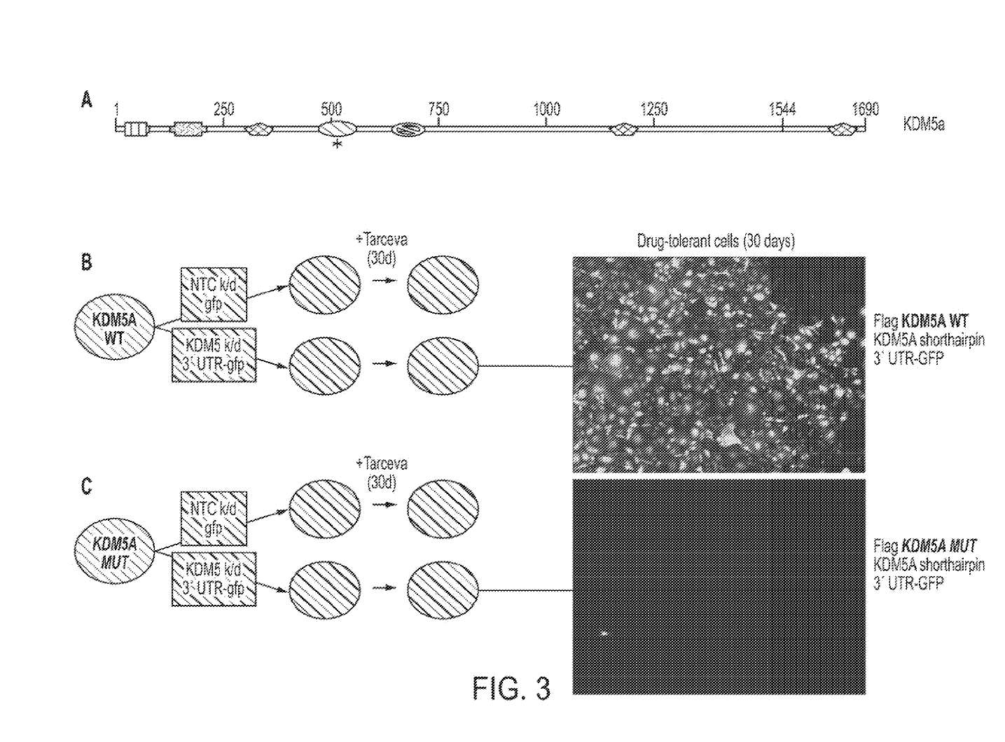

As shown in FIG. 2A, both KDM5A and KDM5B are upregulated in the human non-small-cell-lung cancer line PC9 drug tolerant persisters (DTPs) compared to parental PC9 cells. Further, as shown in FIG. 2B, relative expression levels of KDM5A mRNA is enriched in neoadjuvant lung adenocarcinoma patient samples compared to naïve lung adenocarcinoma patients. Consistent with the change in expression levels of KDM5A and KDM5B, by both Western blotting and MSD ELISA, H3K4 me3 and H3K4 me2 are reduced in PC9 DTP compared to PC9 parental cells as shown in FIGS. 2C-D.

To confirm that KDM5A demethylase activity is required for the establishment of drug-tolerance, the expression of KDM5A shorthairpin with 3′-UTR-GFP knockdown was shown to eliminate PC9 drug tolerant cells. The elimination of PC9 drug tolerant cells by KDM5A shorthairpin with 3′-UTR-GFP knockdown was rescued by co-expression of KDM5A wild-type-FLAG tagged; however, a KDM5A demethylase-catalytically inactive mutant was unable to rescue elimination of PC9 drug tolerant cells by KDM5A shorthairpin with 3′-UTR-GFP knockdown. See FIG. 3B-C. These experiments showed that drug-tolerant cells lose knockdown of endogenous gene, unless wt KDM5a was present.

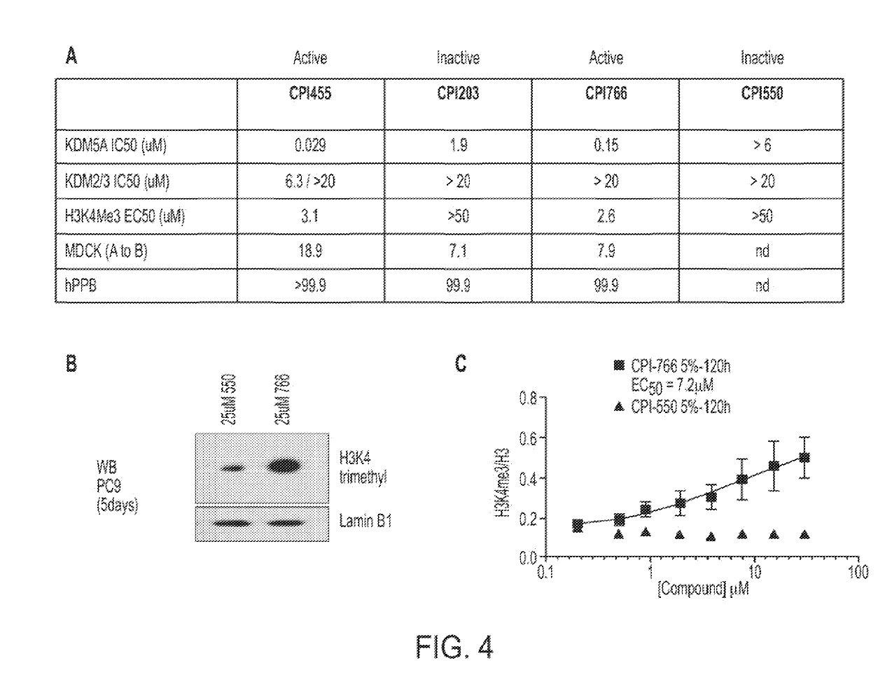

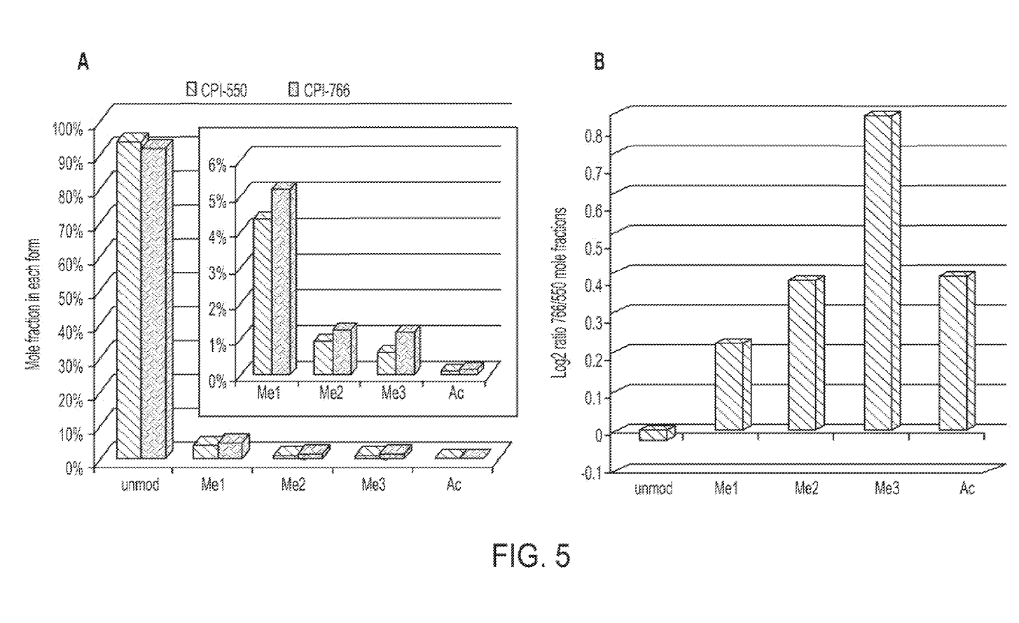

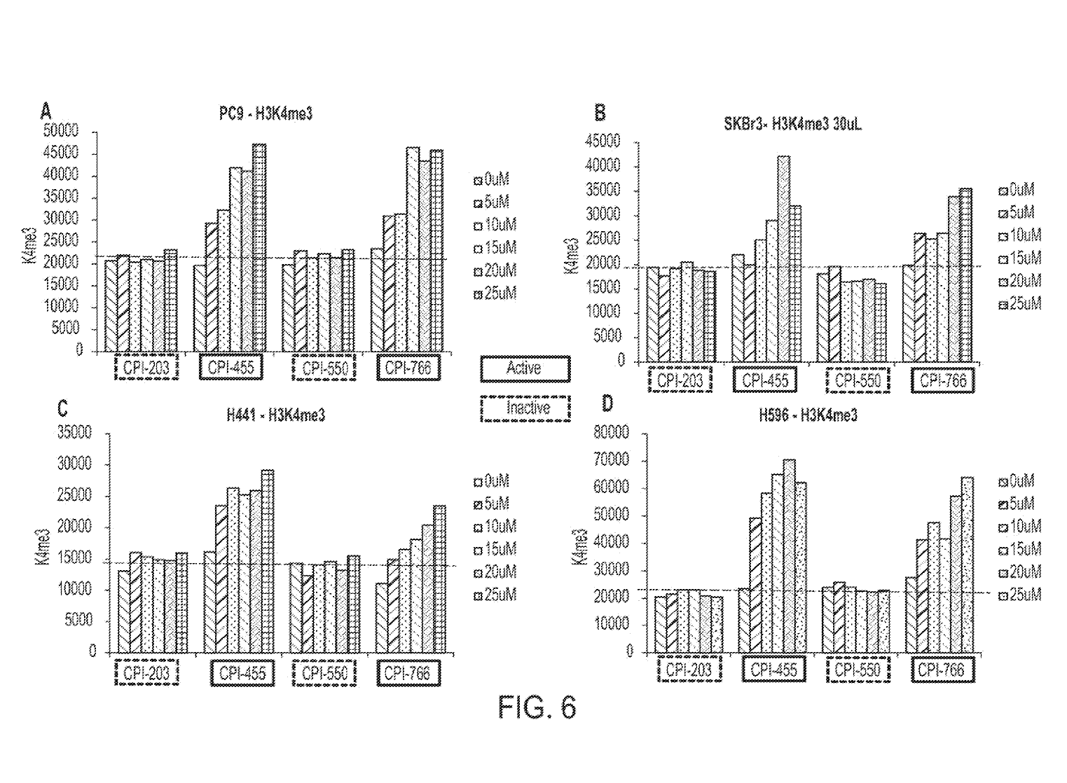

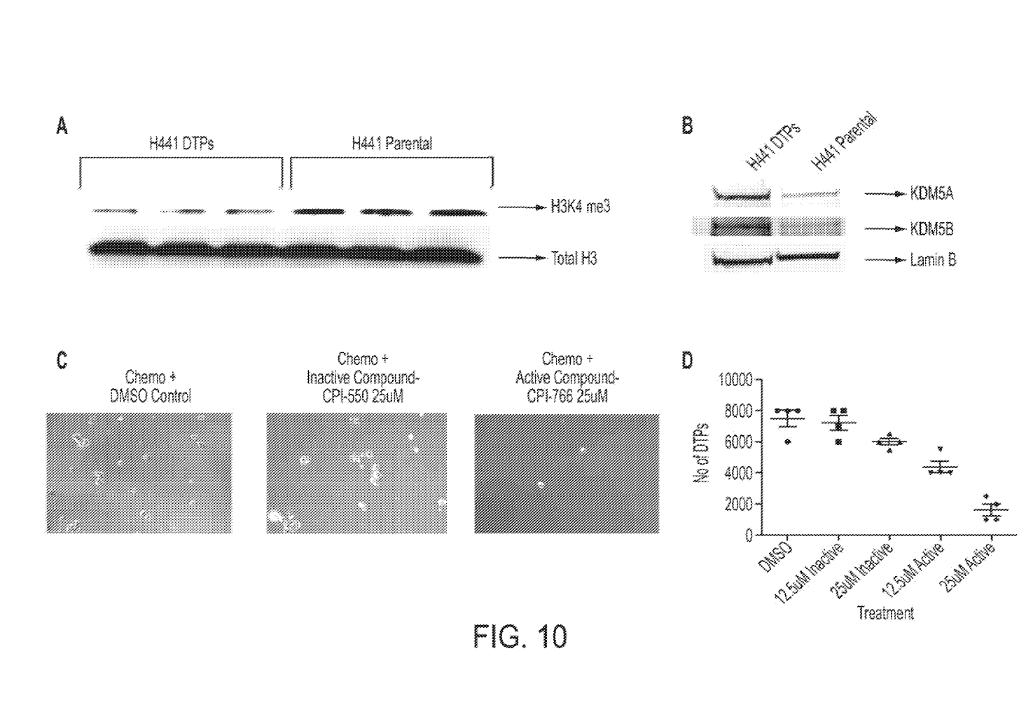

Small molecule KDM5 antagonists as shown in FIG. 4 and described above were capable of inhibiting demethylation of H3K4 and an accumulation of H3K4 me3 was observed by Western blotting, MSD ELISA, and mass spectrometry. See FIG. 4B-C, FIG. 5A-B, FIG. 10, and data not shown. These small molecule KDM5 antagonists, including CPI-455 and PCI-766, increase H3K4me3 in multiple tested models PC9 (NSCLC), SKBR3 (breast cancer), H441 (NSCLC), and H596 (lung epithelial adenosquamous carcinoma) by MSD ELISA as shown in FIGS. 6A-D.

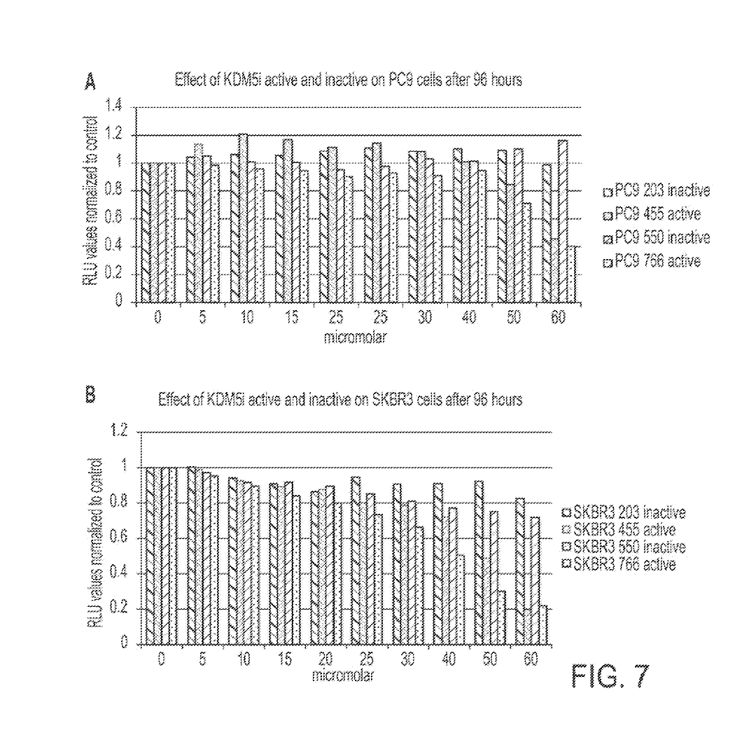

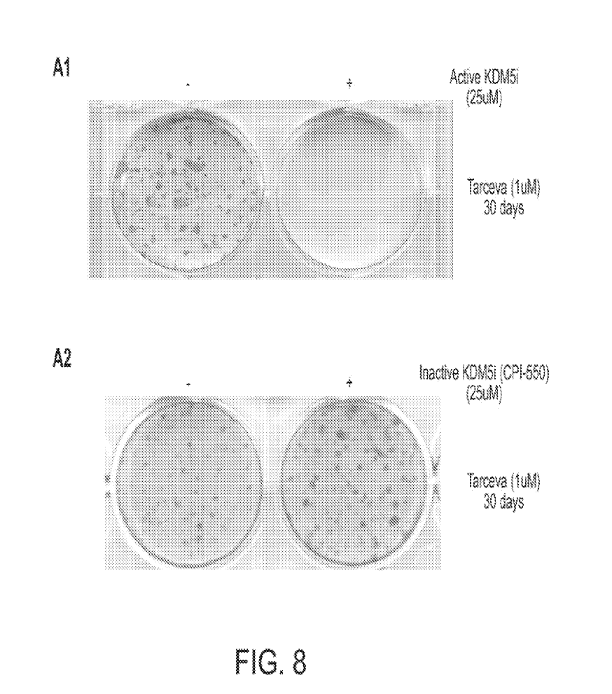

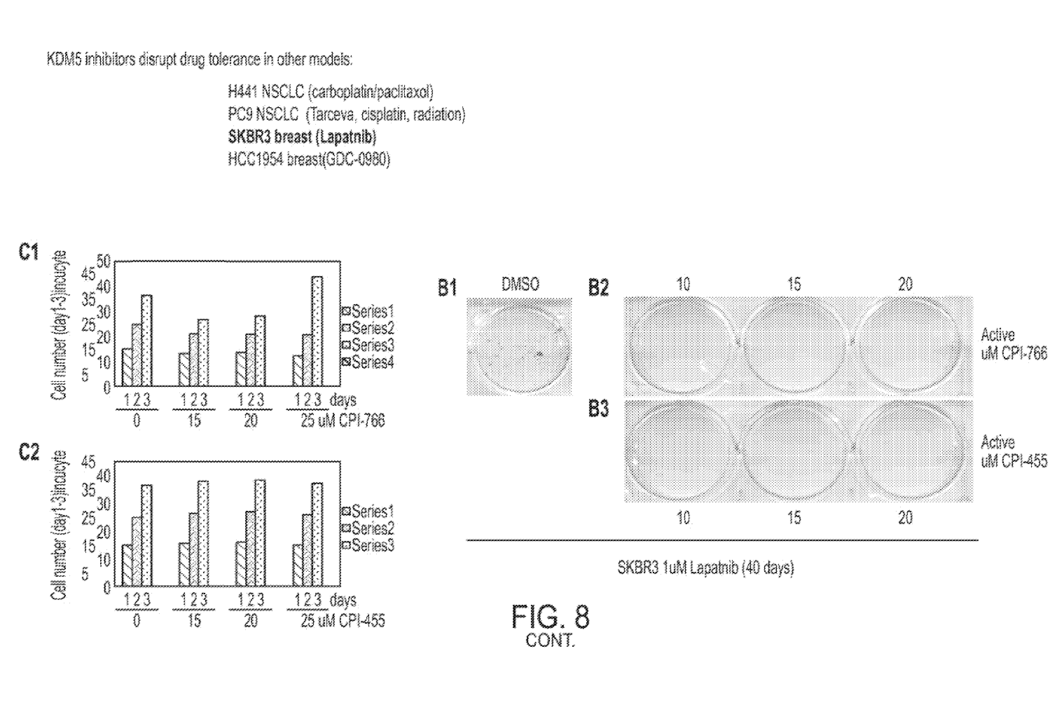

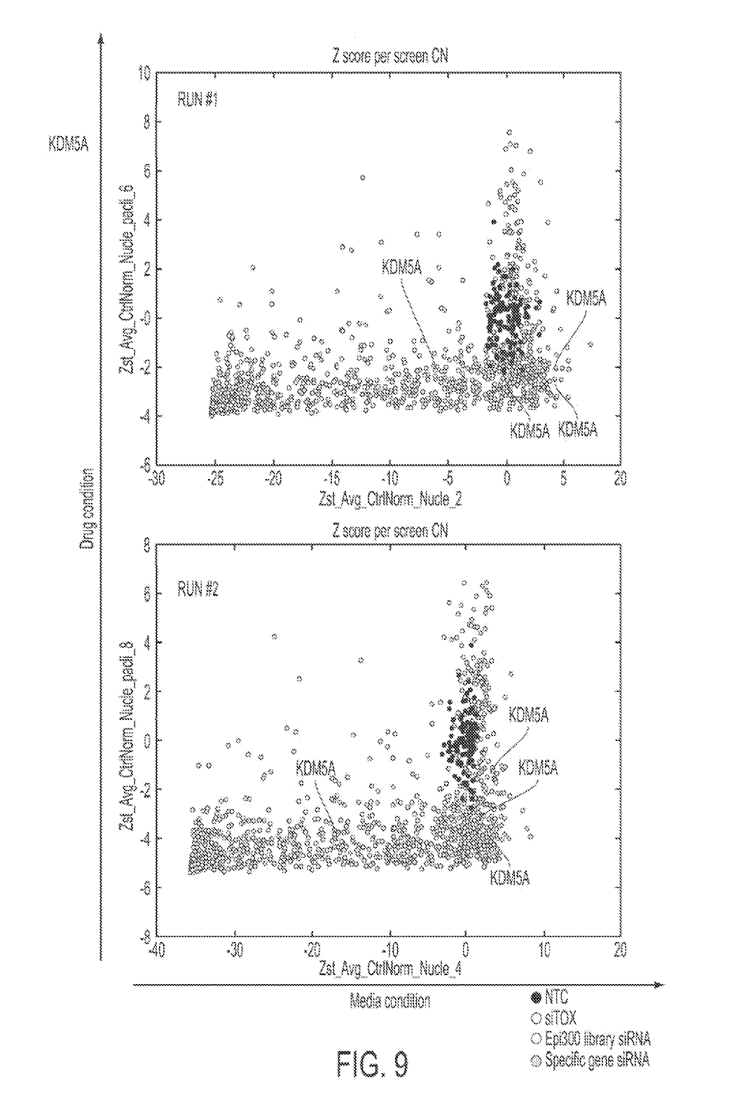

The active small molecule KDM5 antagonists, CPI-455 and CPI-766, alone did not substantially affect cell number as measured after 96 hours in drug at concentrations below 50 uM in PC9 cells and concentrations below 25 uM in SKBR3 as shown in FIGS. 7A-B. However, the small molecule KDM5 antagonists at these concentrations were capable of disrupting drug tolerance when combined with a cancer therapy agent. As shown in FIG. 8 and data not shown, active small molecule KDM5 antagonists including CPI-455 and CPI-766 in combination with the recited cancer therapy agent were capable of inhibiting the development of drug tolerant persisters in PC9, SKBR3, HCC1954 (breast cancer), and H441 cells line. In all cases the KDM5 inhibitor has no effect on the proliferation or survival of the parental population.

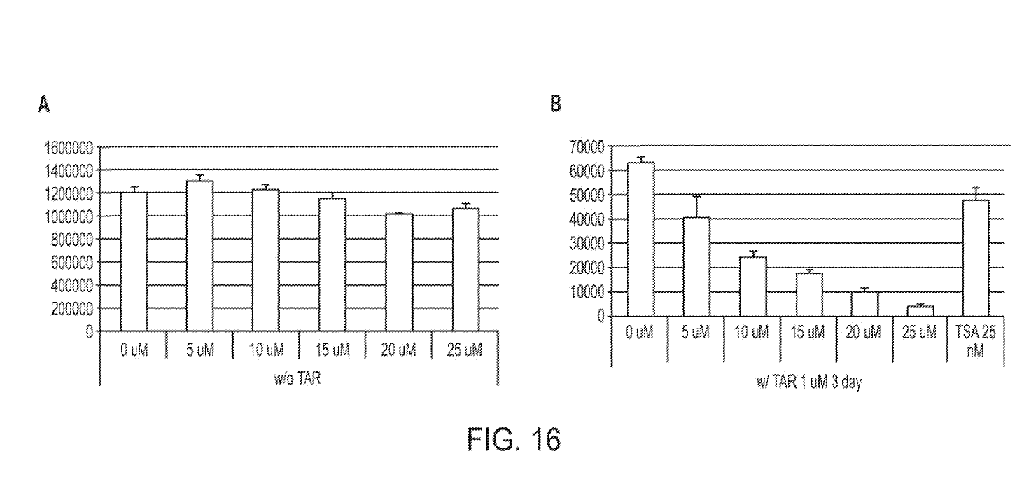

Similarly, using the active small molecule KDM5 antagonist, CPI-382 in combination with erlotinib was capable of reducing the development of drug tolerant persisters in PC9 calls while the inactive control molecule CPI-383 had no significant effect. (see FIG. 16)

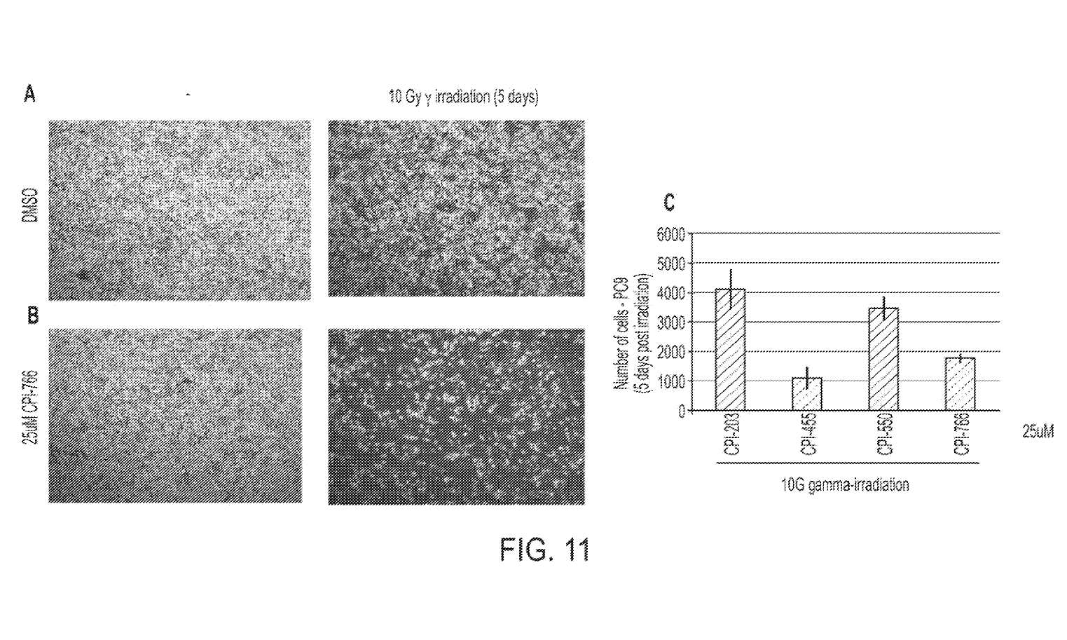

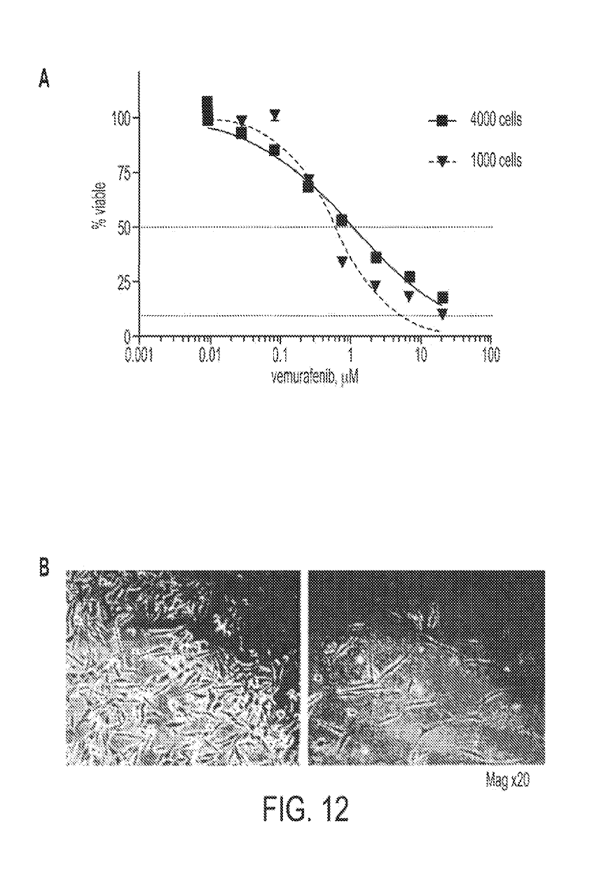

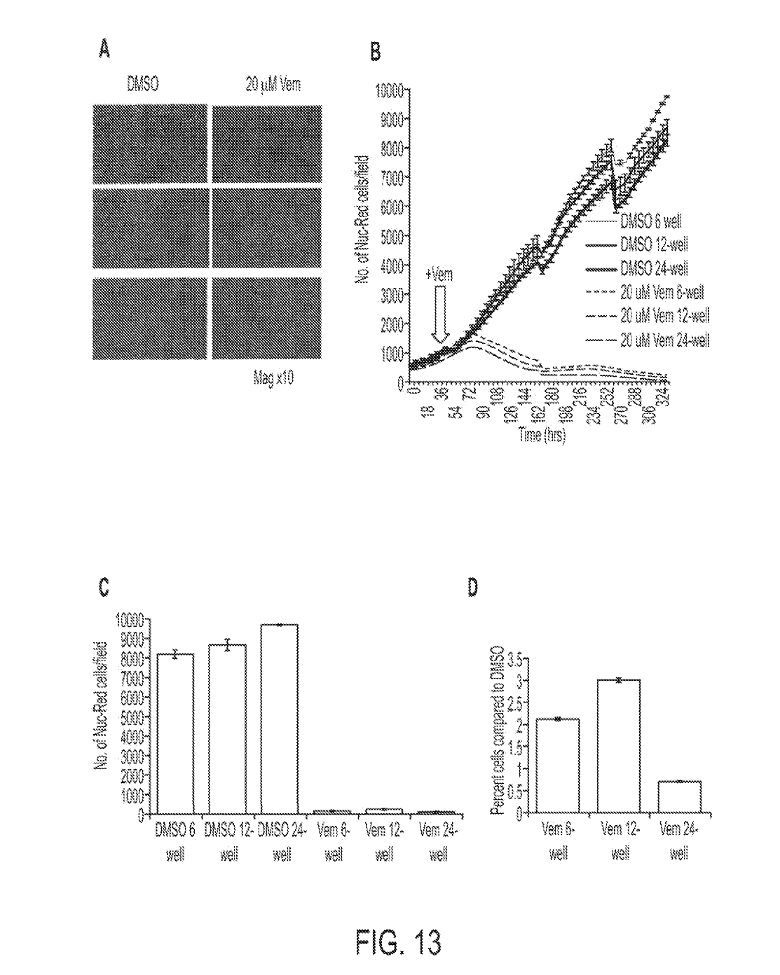

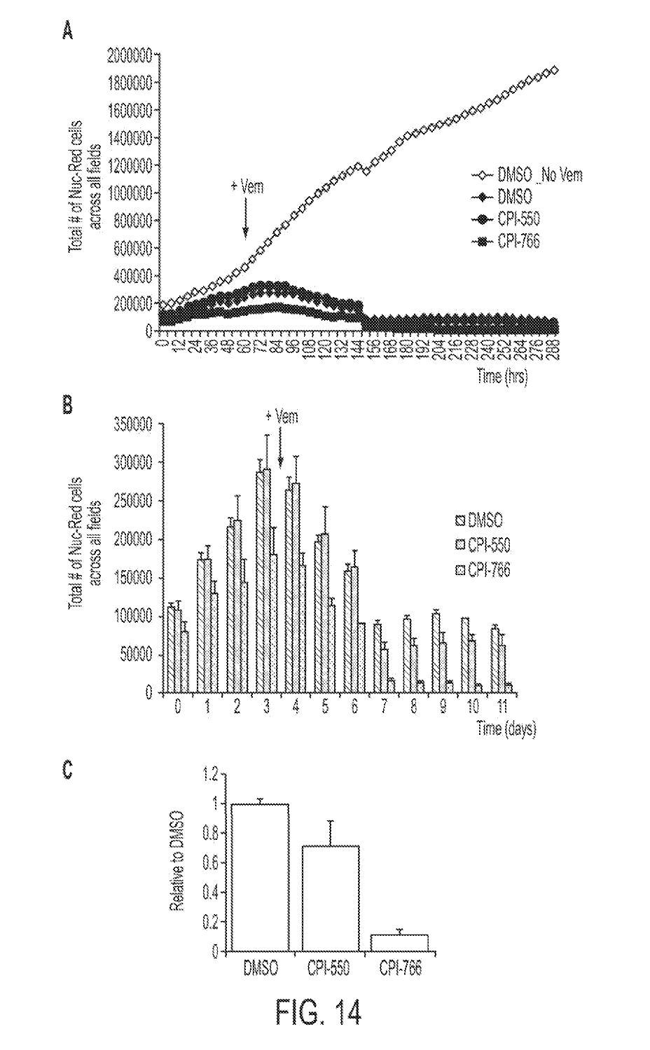

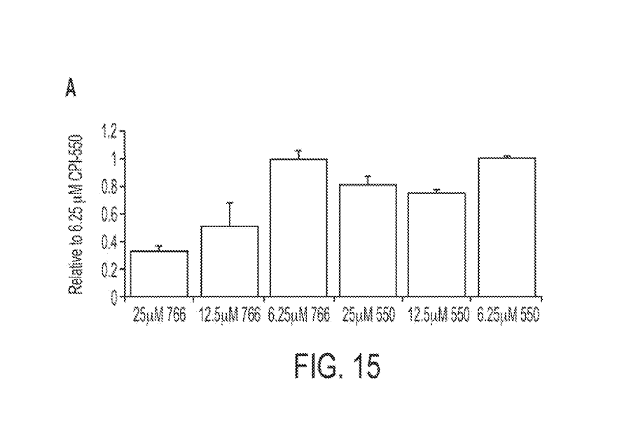

In addition, in the context of radiation therapy as shown in FIG. 11 and data not shown, active small molecule KDM5 antagonists including CPI-455 and CPI-766 in combination with radiation therapy were capable of inhibiting the development of drug tolerant persisters.

These experiments show that the active KDM5 inhibitors show a dose-dependent increase in H3K4me3 as measured by Western blotting, MSD assays, as well as by Mass spectrometry.

>sp|P29375|KDM5A_HUMAN Lysine-specific demethylase 5A OS = Homo sapiens
GN = KDM5A PE = 1 SV = 3
(SEQ ID NO: 1)
MAGVGPGGYAAEFVPPPECPVFEPSWEEFTDPLSFIGRIRPLAEKTGICKIRPPKDWQPPFACE
VKSFRFTPRVQRLNELEAMTRVRLDFLDQLAKFWELQGSTLKIPVVERKILDLYALSKIVASKG
GFEMVTKEKKWSKVGSRLGYLPGKGTGSLLKSHYERILYPYELFQSGVSLMGVQMPNLDLKEKV
EPEVLSTDTQTSPEPGTRMNILPKRTRRVKTQSESGDVSRNTELKKLQIFGAGPKVVGLAMGTK
DKEDEVTRRRKVTNRSDAFNMQMRQRKGTLSVNFVDLYVCMFCGRGNNEDKLLLCDGCDDSYHT
FCLIPPLPDVPKGDWRCPKCVAEECSKPREAFGFEQAVREYTLQSFGEMADNFKSDYFNMPVHM
VPTELVEKEFWRLVSSIEEDVIVEYGADISSKDFGSGFPVKDGRRKILPEEEEYALSGWNLNNM
PVLEQSVLAHINVDISGMKVPWLYVGMCFSSFCWHIEDHWSYSINYLHWGEPKTWYGVPSHAAE
QLEEVMRELAPELFESQPDLLHQLVTIMNPNVLMEHGVPVYRTNQCAGEFVVTFPRAYHSGFNQ
GYNFAEAVNFCTADWLPIGRQCVNHYRRLRRHCVFSHEELIFKMAADPECLDVGLAAMVCKELT
LMTEEETRLRESVVQMGVLMSEEEVFELVPDDERQCSACRTTCFLSALTCSCNPERLVCLYHPT
DLCPCPMQKKCLRYRYPLEDLPSLLYGVKVRAQSYDTWVSRVTEALSANFNHKKDLIELRVMLE
DAEDRKYPENDLFRKLRDAVKEAETCASVAQLLLSKKQKHRQSPDSGRTRTKLTVEELKAFVQQ
LFSLPCVISQARQVKNLLDDVEEFHERAQEAMMDETPDSSKLQMLIDMGSSLYVELPELPRLKQ
ELQQARWLDEVRLTLSDPQQVTLDVMKKLIDSGVGLAPHHAVEKAMAELQELLTVSERWEEKAK
VCLQARPRHSVASLESIVNEAKNIPAFLPNVLSLKEALQKAREWTAKVEAIQSGSNYAYLEQLE
SLSAKGRPIPVRLEALPQVESQVAAARAWRERTGRTFLKKNSSHTLLQVLSPRTDIGVYGSGKN
RRKKVKELIEKEKEKDLDLEPLSDLEEGLEETRDTAMVVAVFKEREQKEIEAMHSLRAANLAKM
TMVDRIEEVKFCICRKTASGFMLQCELCKDWFHNSCVPLPKSSSQKKGSSWQAKEVKFLCPLCM
RSRRPRLETILSLLVSLQKLPVRLPEGEALQCLTERAMSWQDRARQALATDELSSALAKLSVLS
QRMVEQAAREKTEKIISAELQKAAANPDLQGHLPSFQQSAFNRVVSSVSSSPRQTMDYDDEETD
SDEDIRETYGYDMKDTASVKSSSSLEPNLFCDEEIPIKSEEVVTHMWTAPSFCAEHAYSSASKS
CSQGSSTPRKQPRKSPLVPRSLEPPVLELSPGAKAQLEELMMVGDLLEVSLDETQHIWRILQAT
HPPSEDRFLHIMEDDSMEEKPLKVKGKDSSEKKRKRKLEKVEQLFGEGKQKSKELKKMDKPRKK
KLKLGADKSKELNKLAKKLAKEEERKKKKEKAAAAKVELVKESTEKKREKKVLDIPSKYDWSGA
EESDDENAVCAAQNCQRPCKDKVDWVQCDGGCDEWFHQVCVGVSPEMAENEDYICINCAKKQGP
VSPGPAPPPSFIMSYKLPMEDLKETS
>sp|Q9UGL1|KDM5B_HUMAN Lysine-specific demethylase 5B OS = Homo
sapiens GN = KDM5B PE = 1 SV = 3
(SEQ ID NO: 2)
MEAATTLHPGPRPALPLGGPGPLGEFLPPPECPVFEPSWEEFADPFAFIHKIRPIAEQTGICKV
RPPPDWQPPFACDVDKLHFTPRIQRLNELEAQTRVKLNFLDQTAKYWELQGSTLKIPHVERKIL
DLFQLNKLVAEEGGFAVVCKDRKWTKIATKMGFAPGKAVGSHIRGHYERILNPYNLFLSGDSLR
CLQKPNLTTDTKDKEYKPHDIPQRQSVQPSETCPPARRAKRMRAEAMNIKIEPEETTEARTHNL
RRRMGCPTPKCENEKEMKSSIKQEPIERKDYIVENEKEKPKSRSKKATNAVDLYVCLLCGSGND
EDRLLLCDGCDDSYHTFCLIPPLHDVPKGDWRCPKCLAQECSKPQEAFGFEQAARDYTLRTFGE
MADAFKSDYFNMPVHMVPTELVEKEFWRLVSTIEEDVTVEYGADIASKEFGSGFPVRDGKIKLS
PEEEEYLDSGWNLNNMPVMEQSVLAHITADICGMKLPWLYVGMCFSSFCWHIEDHWSYSINYLH
WGEPKTWYGVPGYAAEQLENVMKKLAPELFVSQPDLLHQLVTIMNPNTLMTHEVPVYRTNQCAG
EFVITFPRAYHSGFNQGFNFAEAVNFCTVDWLPLGRQCVEHYRLLHRYCVFSHDEMICKMASKA
DVLDVVVASTVQKDMAIMIEDEKALRETVRKLGVIDSERMDFELLPDDERQCVKCKTTCFMSAI
SCSCKPGLLVCLHHVKELCSCPPYKYKLRYRYTLDDLYPMMNALKLRAESYNEWALNVNEALEA
KINKKKSLVSFKALIEESEMKKFPDNDLLRHLRLVTQDAEKCASVAQQLLNGKRQTRYRSGGGK
SQNQLTVNELRQFVTQLYALPCVLSQTPLLKDLLNRVEDFQQHSQKLLSEETPSAAELQDLLDV
SFEFDVELPQLAEMRIRLEQARWLEEVQQACLDPSSLTLDDMRRLIDLGVGLAPYSAVEKAMAR
LQELLTVSEHWDDKAKSLLKARPRHSLNSLATAVKEIEEIPAYLPNGAALKDSVQRARDWLQDV
EGLQAGGRVPVLDTLIELVTRGRSIPVHLNSLPRLETLVAEVQAWKECAVNTFLTENSPYSLLE
VLCPRCDIGLLGLKRKQRKLKEPLPNGKKKSTKLESLSDLERALTESKETASAMATLGEARLRE
MEALQSLRLANEGKLLSPLQDVDIKICLCQKAPAAPMIQCELCRDAFHTSCVAVPSISQGLRIW
LCPHCRRSEKPPLEKILPLLASLQRIRVRLPEGDALRYMIERTVNWQHRAQQLLSSGNLKFVQD
RVGSGLLYSRWQASAGQVSDTNKVSQPPGTTSFSLPDDWDNRTSYLHSPFSTGRSCIPLHGVSP
EVNELLMEAQLLQVSLPEIQELYQTLLAKPSPAQQTDRSSPVRPSSEKNDCCRGKRDGINSLER
KLKRRLEREGLSSERWERVKKMRTPKKKKIKLSHPKDMNNFKLERERSYELVRSAETHSLPSDT
SYSEQEDSEDEDAICPAVSCLQPEGDEVDWVQCDGSCNQWFHQVCVGVSPEMAEKEDYICVRCT
VKDAPSRK
>sp|P41229|KDM5C_HUMAN Lysine-specific demethylase 50 OS = Homo
sapiens GN = KDM5C PE = 1 SV = 2
(SEQ ID NO: 3)
MEPGSDDFLPPPECPVFEPSWAEFRDPLGYIAKIRPIAEKSGICKIRPPADWQPPFAVEVDNFR
FTPRIQRLNELEAQTRVKLNYLDQIAKFWEIQGSSLKIPNVERRILDLYSLSKIVVEEGGYEAI
CKDRRWARVAQRLNYPPGKNIGSLLRSHYERIVYPYEMYQSGANLVQCNTRPFDNEEKDKEYKP
HSIPLRQSVQPSKFNSYGRRAKRLQPDPEPTEEDIEKNPELKKLQIYGAGPKMMGLGLMAKDKT
LRKKDKEGPECPPTVVVKEELGGDVKVESTSPKTFLESKEELSHSPEPOTKMTMRLRRNHSNAQ
FIESYVCRMCSRGDEDDKLLLCDGCDDNYHIFCLLPPLPEIPKGVWRCPKCVMAECKRPPEAFG
FEQATREYTLQSFGEMADSFKADYFNMPVHMVPTELVEKEFWRLVNSIEEDVTVEYGADIHSKE
FGSGFPVSDSKRHLTPEEEEYATSGWNLNVMPVLEQSVLCHINADISGMKVPWLYVGMVFSAFC
WHIEDHWSYSINYLHWGEPKTWYGVPSLAAEHLEEVMKKLTPELFDSQPDLLHQLVTLMNPNTL
MSHGVPVVRTNQCAGEFVITFPRAYHSGFNQGYNFAEAVNFCTADWLPAGRQCIEHYRRLRRYC
VFSHEELICKMAACPEKLDLNLAAAVHKEMFIMVQEERRLRKALLEKGITEAEREAFELLPDDE
RQCIKCKTTCFLSALACYDCPDGLVCLSHINDLCKCSSSRQYLRYRYTLDELPAMLHKLKVRAE
SFDTWANKVRVALEVEDGRKRSLEELRALESEARERRFPNSELLQQLKNCLSEAEACVSRALGL
VSGQEAGPHRVAGLQMTLTELRAFLDQMNNLPCAMHQIGDVKGVLEQVEAYQAEAREALASLPS
SPGLLQSLLERGRQLGVEVPEAQQLQRQVEQARWLDEVKRTLAPSARRGTLAVMRGLLVAGASV
APSPAVDKAQAELQELLTIAERWEEKAHLCLEARQKHPPATLEAIIREAENIPVHLPNIQALKE
ALAKARAWIADVDEIQNGDHYPCLDDLEGLVAVGRDLPVGLEELRQLELQVLTAHSWREKASKT
FLKKNSCYTLLEVLCPCADAGSDSTKRSRWMEKELGLYKSDTELLGLSAQDLRDPGSVIVAFKE
GEQKEKEGILQLRRTNSAKPSPLASSSTASSTTSICVCGQVLAGAGALQCDLCQDWFHGRCVSV
PRLLSSPRPNPTSSPLLAWWEWDTKFLCPLCMRSRRPRLETILALLVALQRLPVRLPEGEALQC
LTERAISWQGRARQALASEDVTALLGRLAELRQRLQAEPRPEEPPNYPAAPASDPLREGSGKDM
PKVQGLLENGDSVTSPEKVAPEEGSGKRDLELLSSLLPQLTGPVLELPEATRAPLEELMMEGDL
LEVTLDENHSIWQLLQAGQPPDLERIRTLLELEKAERHGSRARGRALERRRRRKVDRGGEGDDP
AREELEPKRVRSSGPEAEEVQEEEELEEETGGEGPPAPIPTTGSPSTQENQNGLEPAEGTTSGP
SAPFSTLTPRLHLPCPQQPPQQQL
sp|Q9BY66|KDM5D_HUMAN Lysine-specific demethylase 5D OS = Homo
sapiens GN = KDM5D PE = 1 SV = 2
(SEQ ID NO: 4)
MEPGCDEFLPPPECPVFEPSWAEFQDPLGYIAKIRPIAEKSGICKIRPPADWQPPFAVEVDNFR
FTPRVQRLNELEAQTRVKLNYLDQIAKFWEIQGSSLKIPNVERKILDLYSLSKIVIEEGGYEAI
CKDRRWARVAQRLHYPPGKNIGSLLRSHYERIIYPYEMFQSGANHVQCNTHPFDNEVKDKEYKP
HSIPLRQSVQPSKFSSYSRRAKRLQPDPEPTEEDIEKHPELKKLQIYGPGPKMMGLGLMAKDKD
KTVHKKVTCPPTVTVKDEQSGGGNVSSTLLKQHLSLEPCTKTTMQLRKNHSSAQFIDSYICQVC
SRGDEDDKLLFCDGCDDNYHIFCLLPPLPEIPRGIWRCPKCILAECKQPPEAFGFEQATQEYSL
QSFGEMADSFKSDYFNMPVHMVPTELVEKEFWRLVSSIEEDVTVEYGADIHSKEFGSGFPVSNS
KQNLSPEEKEYATSGWNLNVMPVLDQSVLCHINADISGMKVPWLYVGMVFSAFCWHIEDHWSYS
INYLHWGEPKTWYGVPSLAAEHLEEVMKMLTPELFDSQPDLLHQLVTLMNPNTLMSHGVPVVRT
NQCAGEFVITFPRAYHSGFNQGYNFAEAVNFCTADWLPAGRQCIEHYRRLRRYCVFSHEELICK
MAAFPETLDLNLAVAVHKEMFIMVQEERRLRKALLEKGVTEAEREAFELLPDDERQCIKCKTTC
FLSALACYDCPDGLVCLSHINDLCKCSSSRQYLRYRYTLDELPTMLHKLKIRAESFDTWANKVR
VALEVEDGRKRSFEELRALESEARERRFPNSELLQRLKNCLSEVEACIAQVLGLVSGQVARMDT
PQLTLTELRVLLEQMGSLPCAMHQIGDVKDVLEQVEAYQAEAREALATLPSSPGLLRSLLERGQ
QLGVEVPEAHQLQQQVEQAQWLDEVKQALAPSAHRGSLVIMQGLLVMGAKIASSPSVDKARAEL
QELLTIAERWEEKAHFCLEARQKHPPATLEAIIRETENIPVHLPNIQALKEALTKAQAWIADVD
EIQNGDHYPCLDDLEGLVAVGRDLPVGLEELRQLELQVLTAHSWREKASKTFLKKNSCYTLLEV
LCPCADAGSDSTKRSRWMEKALGLYQCDTELLGLSAQDLRDPGSVIVAFKEGEQKEKEGILQLR
RTNSAKPSPLAPSLMASSPTSICVCGQVPAGVGVLQCDLCQDWFHGQCVSVPHLLTSPKPSLTS
SPLLAWWEWDTKFLCPLCMRSRRPRLETILALLVALQRLPVRLPEGEALQCLTERAIGWQDRAR
KALASEDVTALLRQLAELRQQLQAKPRPEEASVYTSATACDPIREGSGNNISKVQGLLENGDSV
TSPENMAPGKGSDLELLSSLLPQLTGPVLELPEAIRAPLEELMMEGDLLEVTLDENHSIWQLLQ
AGQPPDLDRIRTLLELEKFEHQGSRTRSRALERRRRRQKVDQGRNVENLVQQELQSKRARSSGI
MSQVGREEEHYQEKADRENMFLTPSTDHSPFLKGNQNSLQHKDSGSSAACPSLMPLLQLSYSDE
QQL

### Example 2

### siRNA Screening Methods

Cells were reverse transfected in black 96 well clear bottom plates (Corning, catalog #3603) at 1000 cell per well using 0.0625 ul of DharmaFECT 1 transfection lipid (Dharmacon, catalog #T-2001) and single siRNA (Dharmacon siGENOME) at 12.5 nM final concentration. Cells were subsequently transfected for 48-72 hours before replacing the transfection media by either 1 uM relevant drug treatment in media or media alone. After 72 hours of incubation the media+/−drug was then replaced with fresh media to enable recovery of the drug tolerant persisters (DTPs) that survived after the relevant drug treatment (recovery phase). After 3 days recovery phase, final cell viability was measured using CyQUANT Direct cell proliferation assay (Molecular Probes) according to the manufacturer protocol. CyQUANT fluorescent signal was detected using a GE IN Cell Analyzer 2000 (4× objective) and quantified as number of cell per well using an image analysis algorithm developed using GE Developer Tollbox 1.9.1. Screening data were subsequently processed in Microsoft Excel. The entire Epi300 siRNA screen was run on each cell line twice in completely independent conditions.

The following siRNA sequences were used.

| | | | | Entrez |
|---|---|---|---|---|
| | | Gene | RefSeq | Gene |
| Type | Sequence | Symbol | Accession | ID |
| siRNA | GCAAAUGAGACAACGGAAA | KDM5A | NM_001042603 | 5927 |
| | (SEQ ID NO: 11) | | | |
| siRNA | UGACAAUGGUGGACCGCAU | KDM5A | NM_001042603 | 5927 |
| | (SEQ ID NO: 12) | | | |
| siRNA | CAACACAUAUGGCGGAUUU | KDM5A | NM_001042603 | 5927 |
| | (SEQ ID NO: 13) | | | |
| siRNA | GGAUGAACAUUCUGCCGAA | KDM5A | NM_001042603 | 5927 |
| | (SEQ ID NO: 14) | | | |

### Results:

H1299 DTP cells were prepared and screened as described above using the taxane, paclitaxel, as the drug. KDM5A siRNAs as shown in FIG. 6 substantially reduced H1299 DTP viability in the presence of paclitaxel compared to media alone.

### Example 3

### Use of Melanoma DTP Model to Study the Role of KDM5 Inhibitors in DTP Formation

Melanoma is the less common but most dangerous form of skin cancer that causes most of the skin cancer related deaths. In the US, about 160,000 new cases of melanoma are diagnosed every year, of which more than half are invasive melanomas (American Cancer Society. Cancer Facts & FIGS. 2014. Atlanta, Ga.: American Cancer Society; 2014) One of the recent developments in the treatment of melanoma has come from the use of targeted chemotherapeutic vemurafenib (Chapman et al. N Engl J. Med 2011; 364:2507-2516). This drug only works in melanoma patients whose cancer has a V600E BRAF mutation. This mutation occurs in about half of melanoma patients and initial response to the drug is good but as with a lot of other agents, overtime, cells develop resistance to this therapy and no longer respond to the treatment.

In order to understand mechanisms of drug resistance, melanoma cell lines were utilized. 18 melanoma cell lines, both wt and V600E mutants, were screened to identify vemurafenib-sensitive cell lines. The results showed that the mutant cell lines were sensitive to vemurafenib. 3 different mutant cell lines were selected (A375, HT144 and Colo-829) to establish drug tolerant persistors (DTP's). Of the three cell lines tested, Colo-829 showed a consistent DTP phase, and the cell line was easy to work with. Thus, this cell line was selected as the model of choice for DTP formation. Next, extensive assay development was performed in order to perform DTP assays in a semi high throughput manner. The steps included generation of colo-829 cells that constitutively express a red-fluorescent marker in the nucleus (Nuc-Red) cells that helped acquire images from each cell using incucyte Zoom (Essen Bio) and testing various plate formats to choose the plates with maximum number of wells without compromising that data consistency. Once the assay was developed, experiments were performed using KDM5 inhibitors to determine if there were any effects of KDM5 inhibition on DTP formation in these cells. The results clearly showed that pre-treatment with active KDM5 inhibitor significantly reduced the number of DTP's formed upon vemurafenib treatment.

In conclusion, Colo-829 is a representative model system to study DTP formation in melanoma cell lines, with findings similar to other DTP models, and KDM5 inhibitors play a significant role in abrogating the DTP population in this melanoma model.

### Materials and Methods

1. Selection of the Melanoma Cell Line for DTP Establishment

One goal was to identify a cell line that would be sensitive to vemurafenib and amenable to DTP formation. For this, a set of 18 melanoma cell lines were tested by standard cell viability assay using cell titer Glo readout after 4 days of incubation with 8 different doses of vemurafenib. The results identified 3 melanoma cell lines (A375, Colo829 and HT144) with GI50's below 600 nM. All 3 of these cell lines were b raf mutant. After experiments with all these cell lines, Colo-829 was selected as the preferred cell line for DTP establishment.

Methods

Compound: vemurafenib was used (Plx-4032, Selleck Chemicals)

1.1 Identifying melanoma cell lines sensitive to Vemurafenib
Day 0: 1000 cells/well were plated in 100 μl total volume in a 96 well flat bottom plate.
Day 1: The cells were treated with vemurafenib. The highest concentration in the assay was 20 μM and was diluted 3 fold down with the lowest concentration being 9 nM.
Day 5: 100 μl of cell titer glo was added to each well and the plates read on Envision. The data was plotted using graphpad prism. 1.2 DTP establishment in melanoma cell lines
Day 0: Cells (4.5×106 cells/P150 dishes) were plated, 4 plates/cell line.
Day 2: All the cell lines were 60-80% confluent. 2 plates were treated for each cell line with 20 μM Vemurafenib and the 2 remaining plates with DMSO (control).
Day 5: Media was changed. Cells in the DMSO treated plates were confluent. The cells in these plates were counted and ⅛th the numbers were replated in 2 new P150 dishes. All other plates were treated with vemurafenib.
Day 8: Media was changed: The plates were treated with vemurafenib or DMSO, respectively.
Day 11: The cells were washed with PBS and trypsinise. The cells were counted and the percentage of cells remaining on the Vemurafenib treated dish vs the control dish were identified. The cells remaining on the Vemurafenib treated dishes were called drug tolerant persistors (DTP).

2. Assay Development to Run the DTP Assay in a Semi High Throughput Format

2.1 Preparing NucLight Red Positive Colo-829 Cells

Material: CellPlayer™ NucLight Red (Lenti, EF-1 alpha, bleo) from Essen Bio.

100K cells were plated in a 6 well plate and the following day the NucLight Red virus was added at an MOI of 1. The cells were subsequently selected in zeocin and the Nuc-red cells were routinely maintained in zeocin.

2.2 Testing DTP Formation in 6, 12 and 24 Well Formats

1×105 cells/ml Nuc-red colo-829 cells were plated in 6—(3 ml), 12—(2 ml) and 24—(1 ml) well plates respectively. After 2 days, vemurafenib (20 μM) or DMSO (control) was added to replicate wells and the DTP formation assay was performed as described earlier.

3. Testing KDM5 Inhibitors in the DTP Assay

Compounds used: CPI-766 (active) and CPI-550 (inactive)

Day 0: 2×106 Colo-829 cells were plated in 10 cm dishes.

Day 1: Cells were treated with 25 μM of CPI-766 or CPI-550 or DMSO.

Day 3: These KDM5 inhibitor pretreated cells were plated in quadruplicates onto 12 well plates (2×105 cells per well, 2 ml volume) and the respective KDM5 inhibitors added. The plates were placed in the incucyte to start collecting the data.

Day 5: The cells were treated with fresh KDM5 inhibitors. Half the number of wells were treated with 20 μM vemurafenib and DMSO was added to the remaining wells.

Day 8 and Day 11: The treatment was repeated with the compounds as on Day 5.

Day 14: The experiment was completed.

### Example 4

### KDM5 Inhibitor Blocks Drug Tolerance of a Colorectal Cancer Cell Line

A model for resistance to standard-of-care chemotherapy for colorectal cancer (CRC) was developed by treating CRC cell line SW480 with a combination of 5-fluorouracil and the irinotecan active metabolite SN-38 in a ratio of their relative IC₅₀ values (33 μM 5-FU and 6 ηM SN-38) continuously for 16 days, followed by drug withdrawal. The cells progressively die during this treatment period. The remaining DTP cells constitute approximately 8% of the initial cell population and appear large and non-dividing. After drug withdrawal, cells continue to die for several more days, but then re-initiate proliferation and expand to form DTEP colonies by about 2 weeks in the absence of drugs.

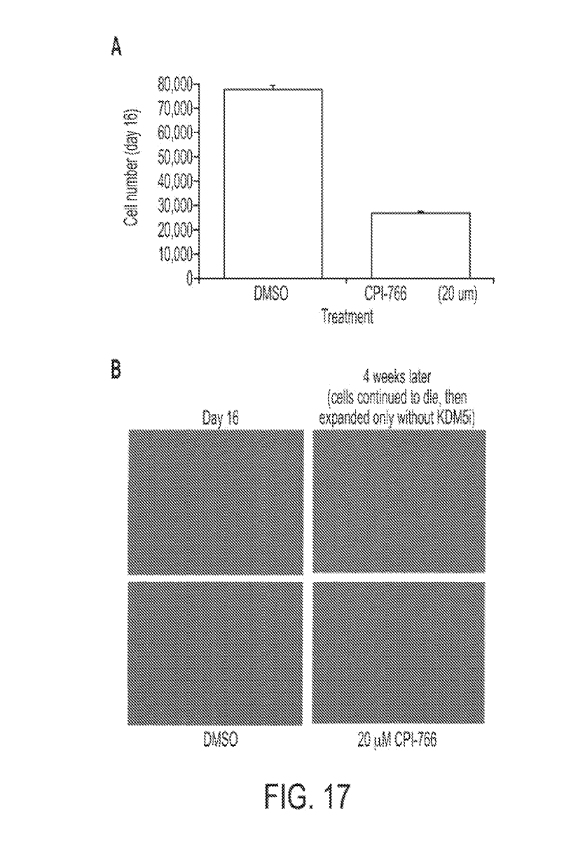

As illustrated in FIG. 17, pre-treating SW480 cells with KDM5 inhibitor CPI-766 at 20 μM for 7 days before chemotherapeutic treatment results in a 2.9-fold decrease in DTP cell survival assayed at day 16. Moreover, the KDM5 inhibitor completely inhibits DTP cell expansion since surviving cells at day 16 eventually all die after drug withdrawal on this day and never form colonies. CPI-766 at this concentration does not detectably affect cell survival or proliferation of the parental SW480 cell line for at least 27 days of treatment in the absence of chemotherapeutics. The inactive analog CPI-550 at 20 μM does not affect DTP survival, nor do inhibitors of other chromatin regulators tested (e.g., 0.2 μM 5-azacytidine and 20 ηM Trichostatin A). These results suggest that KDM5 activity is specifically required for survival of the DTP population of SW480 cells during treatment with chemotherapeutics, and for the expansion of these cells after drug withdrawal.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating cancer in an individual comprising administering to the individual (a) an antagonist of KDM5 and (b) a cancer therapy agent.

2. The method of claim 1, wherein the respective amounts of the antagonist of KDM5 and the cancer therapy agent are effective to increase the period of cancer sensitivity and/or delay the development of cell resistance to the cancer therapy agent.

3-10. (canceled)

11. The method of claim 1, wherein the antagonist of KDM5 is an antibody inhibitor, a binding small molecule inhibitor, a binding polypeptide inhibitor, and/or a polynucleotide antagonist.

12. The method of claim 11, wherein the antagonist of KDM5 binds KDM5 and inhibits KDM5 demethylase activity.

13. The method of claim 1, wherein the KDM5 is one or more of KDM5A, KDM5B, KDM5C, and KDM5D.

14. The method of claim 13, wherein the KDM5 is one or more of KDM5A and/or KDM5B.

15. The method of claim 13, wherein the KDM5 is KDM5A and KDM5B.

16. The method of claim 13, wherein the KDM5 is KDM5B.

17. The method of claim 13, wherein the KDM5 is KDM5A, KDM5B, KDM5C, and KDM5D.

18. The method of claim 1, wherein the cancer therapy agent is chemotherapy.

19. The method of claim 1, wherein the cancer therapy agent is chemotherapy and the chemotherapy comprises a taxane.

20. The method of claim 19, wherein the taxane is paclitaxel or docetaxel.

21. The method of claim 1, wherein the cancer therapy agent is chemotherapy and the chemotherapy comprises a platinum agent.

22. The method of claim 1, wherein the cancer therapy agent is a targeted therapy.

23. The method of claim 1, wherein the cancer therapy agent is a targeted therapy and the targeted therapy comprises an antagonist of EGFR.

24. The method of claim 23, wherein the antagonist of EGFR is N-(3-ethynylphenyl)-6,7-bis(2-methoxyethoxy)quinazolin-4-amine or a pharmaceutically acceptable salt thereof (e.g., erlotinib).

25. The method of claim 22, wherein the cancer therapy agent is a targeted therapy and the targeted therapy is a RAF inhibitor.

26. The method of claim 25, wherein the RAF inhibitor is a BRAF and/or CRAF inhibitor.

27. The method of claim 25, wherein the RAF inhibitor is vemurafenib.

28. The method of claim 1, wherein the cancer therapy agent is a targeted therapy and the targeted therapy is a PI3K inhibitor.

29. The method of claim 1, wherein the antagonist of KDM5 is a small molecule KDM5 antagonist.

30. The method of claim 1, wherein the antagonist of KDM5 and the cancer therapy agent are administered concomitantly.

31. The method of claim 1, wherein the antagonist of KDM5 is administered prior to and/or concurrently with the cancer therapy agent.

32. The method of claim 1, wherein the cancer is lung cancer (e.g., non-small cell lung cancer (NSCLC), melanoma, colorectal cancer, pancreatic cancer, and/or breast cancer).
